(19) United States
(12) Patent Application Publication
(10) Pub. No.: US 2022/0396789 A1
(43) Pub. Date: Dec. 15, 2022

(54) SEQUENCE-CONTROLLED POLYMER STORAGE

(71) Applicant: Massachusetts Institute of Technology, Cambridge, MA (US)

(72) Inventors: James L. Banal, Cambridge, MA (US); Mark Bathe, Cambridge, MA (US); Joseph Berleant, Cambridge, MA (US); Charles E. Leiserson, Cambridge, MA (US); Tao Benjamin Schardl, Cambridge, MA (US)

(21) Appl. No.: 17/836,726

(22) Filed: Jun. 9, 2022

Related U.S. Application Data
(60) Provisional application No. 63/208,973, filed on Jun. 9, 2021.

(51) Int. Cl. C12N 15/10

(52) U.S. Cl. CPC C12N 15/1065; C12N 15/1089

(57) ABSTRACT

Disclosed are compositions and methods relating to sequence-controlled storage objected. The disclosed sequence-controlled storage objects can include (a) one or more different sequence-controlled polymers, and (b) a plurality of different feature tags. The sequence-controlled storage object can include (a) one or more different sequence-controlled polymers, and (b) a plurality of different digit tags. Also disclosed are methods of storing desired sequence-controlled polymers as a sequence-controlled storage object, comprising assembling a sequence-controlled storage object from (i) one or more different sequence-controlled polymers, (ii) a plurality of different feature tags, and (iii) optionally one or more encapsulating agents. Also disclosed are methods of automating the assembly of a sequence-controlled storage object comprising using a device with flow.

### CROSS-REFERENCE TO RELATED APPLICATIONS

This application claims priority to and benefit of U.S. Provisional Application No. 63/208,973, filed Jun. 9, 2021. Application No. 63/208,973, filed Jun. 9, 2021, is hereby incorporated herein by reference in its entirety.

### STATEMENT REGARDING FEDERALLY SPONSORED RESEARCH

This invention was made with Government support under Grant Nos. N00014-16-1-2506, N00014-12-1-0621, N00014-18-1-2290, N00014-17-1-2609, N00014-20-1-2084, and N00014-21-1-4013, awarded by the Office of Naval Research (ONR); Grant Nos. CCF1564025, 1729397, CHE1839155, OAC1940231, and CCF1956054, awarded by the National Science Foundation (NSF); Grant No. DE-SC0019998 awarded by the Department of Energy (DOE); Grant No. W911NF-13-D-0001 awarded by the Army Research Office (ARO); and Grant No. FA8750-19-2-1000 awarded by the Air Force Research Laboratory (AFRL). The Government has certain rights in the invention.

### REFERENCE TO SEQUENCE LISTING

The Sequence Listing submitted Jun. 9, 2022, as a text file named “MIT 23164_ST25,” created on May 26, 2022, and having a size of 1,614 bytes is hereby incorporated by reference pursuant to 37 C.F.R. § 1.52(e)(5).

### FIELD OF THE INVENTION

The present invention discloses a method for encapsulation of biomolecules using milli-to-nanoscale capsules, which can be uniquely identified using molecular barcodes, enabling ultradense storage at room temperature.

### BACKGROUND OF THE INVENTION

The central dogma of biology proceeds from DNA to RNA then finally proteins. These biomolecules play critical roles in sustaining life: DNA encodes the information for protein synthesis while RNA carries out the instruction encoded on the DNA. Proteins carry out most biological processes. The explosion and advances of omics technologies have driven the demand to understand individuals' health and predisposition to diseases through the collection, storage, and analyses of DNA, RNA, and proteins. Omics technologies that analyze nucleic acids, i.e., genomics and transcriptomics, are now scientifically advanced and commercialized at scale.

Large-scale storage of nucleic acid samples is critical in basic, translational, and clinical research, synthetic biology foundries, and biodiversity conservation efforts [Ivanova and Kuzmina. Mol Ecol Resour 13, 890-898, doi:10.1111/1755-0998.12134 (2013); Fabre, et al. European Journal of Human Genetics 22, 379-385, doi:10.1038/ejhg.2013.145 (2014)]. Nucleic acid storage requires robust procedures to maintain sample quality, integrity, and function. The current storage temperature requirement for nucleic acids is between 4° C. to −196° C. [Fabre, et al. European Journal of Human Genetics 22, 379-385, doi:10.1038/ejhg.2013.145 (2014); Muller, et al. Biopreserv Biobank 14, 89-98, doi:10.1089/bio.2015.0022 (2016); Miernyk, et al. Biopreserv Biobank 15, 529-534, doi:10.1089/bio.2017.0040 (2017)], where degradation is negligible. However, maintaining such a low temperature for extended periods requires significant energy. Also, large-scale cryogenic storage of nucleic acid materials requires extensive robotics for access, stringent cold-chain management logistics, [Muller, et al. Biopreserv Biobank 14, 89-98, doi:10.1089/bio.2015.0022 (2016); Clermont, et al. Biopreserv Biobank 12, 176-183, doi:10.1089/bio.2013.0082 (2014); Wan, et al. Curr Issues Mol Biol 12, 135-142 (2010)] and redundant copies of samples stored in mirror storage facilities to mitigate the risk of sample loss[Muller, et al. Biopreserv Biobank 14, 89-98, doi:10.1089/bio.2015.0022 (2016)]. Finally, cold storage of nucleic acids in remote or low-resource areas will involve costly measures and complex cold-chain logistics to maintain the integrity and quality of the isolated sample during transport [Clermont, et al. Biopreserv Biobank 12, 176-183, doi:10.1089/bio.2013.0082 (2014)]. A transition towards room-temperature storage from cryogenic storage would reduce energy usage by 40 million kilowatt-hours, which translates to eliminating 18,000 metric tons of annual carbon dioxide emissions and cost savings of $16 million over ten years [Palmer. Nat Med 16, 1056-1057, doi:10.1038/nm1010-1056b (2010)], and 70% reduction of space requirements over cryogenic storage [Lou, et al. Clin Biochem 47, 267-273, doi:10.1016/j.clinbiochem.2013.12.011 (2014)]. The cost and workflow complexity associated with sample processing is also reduced [Lou, et al. Clin Biochem 47, 267-273, doi:10.1016/j.clinbiochem.2013.12.011 (2014)]. Room-temperature storage of nucleic acid samples is achieved either through the addition of stabilizing agents, such as DNAstable® and RNAstable® from Biomatrica, or the use of vacuum canisters, such as DNAshells® and RNAshells® from Imagene. While these room-temperature storage solutions can guarantee nucleic acid stability of 1 year or more, space to store samples and support infrastructure, such as extensive robotic platforms for access and required humidity controls, will still be critical cost considerations [Muller, et al. Biopreserv Biobank 14, 89-98, doi:10.1089/bio.2015.0022 (2016); Lou, et al. Clin Biochem 47, 267-273, doi:10.1016/j.clinbiochem.2013.12.011 (2014)].

While silica particles [Grass, et al. Angewandte Chemie International Edition 54, 2552-2555 (2015); Puddu, et al. Advanced healthcare materials 4, 1332-1338 (2015)], alginates [Gombotz and Wee. Advanced drug delivery reviews 31, 267-285 (1998); Machado, et al. Langmuir 29, 15926-15935 (2013)], and synthetic polymers [Gill and Ballesteros. Trends in biotechnology 18, 282-296 (2000); Zelikin, et al. ACS nano 1, 63-69 (2007)] have been used for storing biomolecules at room temperature, the ability to uniquely identify these storage materials and pool them together to realize an alternative room-temperature storage and retrieval platform for biomolecules has yet to be demonstrated. Programs and functions in DNA-based data storage are described in WO 2021231493 A1.

There is a need for scalable storage of biomolecules that requires little to no energy for maintaining the integrity of samples over 10 years or more.

There is also a need to significantly reduce the footprint required to store biomolecule samples and be able to retrieve thousands to millions of samples rapidly.

Therefore, the object of this invention to provide methods to store and retrieve biomolecules collected from any origin.

It is also the object of this invention to provide methods to encapsulate biomolecules of various lengths and sizes using different chemical and biochemical preparations and different fluidic approaches.

It is also the object of this to provide methods to label encapsulated biomolecules using different fluidic approaches.

It is also the object of this disclosure to provide methods for choosing the barcodes for each particle in such a way as to permit retrieval of collections of particles whose enclosed biomolecules are related by various features including but not restricted to sample type, source, and collection date/time. Barcodes may be selected from an existing pool of sequences designed for optimal properties, such as binding strength and orthogonality.

It is also the object of this disclosure to provide novel methods for designing barcode sequences that permit similarity-based retrieval by permitting probes to bind to multiple distinct barcodes of similar sequence, which label particles whose contained biomolecules are similar under some metrics of interest.

It is a further object of the disclosed invention to provide chemical and biochemical strategies to improve the sorting throughput of barcodes using chemical and biochemical approaches.

It is also an objective of the current invention to provide a biopolymer storage structure, which may include peptides, nucleic acids, or other sequence-controlled polymers, that allows Boolean logic computations.

It is also an objective of the current invention to provide arbitrary nucleic acid origami nanostructures and other nucleic acids and biopolymers as storage blocks, which can be read out either using sequencing or mass spectrometry or other analytical chemical approach.

It is a further objective to provide nucleic acid storage blocks that are capable of forming stable and reconfigurable superstructures for association of storage block structures and position-based storage, as well as parallel computational processing.

It is also an objective to provide nucleic acid storage objects that are capable of accelerated degradation in response to specific external stimuli.

### SUMMARY OF THE INVENTION

Purified nucleic acids from any origin are encapsulated in synthetic packets composed of organic or inorganic polymeric networks. Encapsulation can be performed using automated liquid handling, which mixes the biomolecules of interest with encapsulation reagents, or millifluidic and microfluidic approaches, which traps biomolecules and encapsulation reagents in millimeter to nanometer-sized emulsion reaction containers. The encapsulated biomolecules are then labeled with combinations of orthogonal molecular barcodes identified from a pool of 240,000 [Xu, et al. Proceedings of the National Academy of Sciences 106, 2289-2294, doi:10.1073/pnas.0812506106 (2009)], which uniquely labels and identifies the contents of the sample. The encapsulated biomolecules may also be labeled with non-orthogonal molecular barcodes that permit similarity-based retrieval, such that collections of similar biomolecules may be retrieved simultaneously because a single probe sequence may bind to any one of multiple distinct barcodes of similar sequence. The molecular barcodes may be composed of non-phosphate backbones to improve the stability of strands against nucleases. The process of barcoding can be similarly performed using millifluidic or microfluidic approaches. Upon encapsulation and barcoding, all samples can be collected and pooled into a single vessel. Samples are selected from the pool using complementary probes which may contain optical, chemical, or biochemical tags that can be used as markers for downstream optical or mechanical sorting using millifluidic or microfluidic strategies. Chemical and biochemical reactions can be performed on the barcodes to improve the sorting speed, sorting precision, and limit-of-detection of a specific sorting approach.

Disclosed are compositions and methods relating to sequence-controlled storage objected. The disclosed sequence-controlled storage objects include (a) one or more different sequence-controlled polymers, and (b) a plurality of different feature tags. In some forms, the feature tags are present at the surface of the sequence-controlled storage object. In some forms, each different feature tag corresponds to a single feature attributable to one or more of the different sequence-controlled polymers. In some forms, the single feature to which each different feature tag corresponds is a feature attributable to one or more different of the sequence-controlled polymers. In some forms, the plurality of different feature tags collectively corresponds to a plurality of features that are collectively attributable to the plurality of different sequence-controlled polymers. In some forms, each of the different feature tags is hybridizably distinguishable from all of the other different feature tags.

In some forms, each of the plurality of different feature tags is a member of a different set of feature tags, wherein each set of feature tags corresponds to a set of related features. In some forms, the members of at least one of the sets of feature tags are similarity-encoded feature tags. In some forms, the relative hybridizability of the feature tags in the set is related to the similarity of the features to which the feature tags in the set correspond, wherein feature tags in the set corresponding to more similar features have closer relative hybridizability than feature tags in the set corresponding to less similar features.

In some forms, the similarity encoded feature tags of the set of feature tags were similarity encoded by mapping the features to which the feature tags correspond to an n-dimensional hypercube based on the similarity of the features, wherein n is an integer less than or equal to the number of features to which the feature tags correspond, wherein n is a factor of the number of features to which the feature tags correspond.

In some forms, prior to mapping the features to which the feature tags correspond, the dimensionality of the features to which the feature tags correspond is reduced, wherein the dimensionality-reduced features are mapped to the hypercube based on the similarity of the dimensionality-reduced features.

In some forms, the similarity encoded feature tags of the set of feature tags were similarity encoded by (a) reducing the dimensionality of the features to which the feature tags correspond and (b) mapping the dimensionality-reduced features to an n-dimensional hypercube based on the similarity of the dimensionality-reduced features, wherein n is an integer less than or equal to the number of features to which the feature tags correspond, wherein n is a factor of the number of features to which the feature tags correspond.

In some forms, the number of edges of the hypercube between the nodes to which any two of the mapped features are mapped is proportional to the similarity of the two features. In some forms, the members of at least one of the sets of feature tags are hybridization ordered, wherein the members of the at least one of the sets of feature tags have the same number of nucleotides.

In some forms, in at least one of the sets of feature tags, (a) the members of the set of feature tags have the same number of nucleotides and (b) each of the feature tags in the set differs from one or two other feature tags in the set by 1 to x mismatched nucleotides, wherein the mismatched nucleotides are (i) at least two nucleotides from either end of the feature tag and (ii) are separated by at least one matching nucleotide in the feature tags, and wherein x is the number of different nucleotide positions in the feature tags that are varied in the set.

In some forms, independently for one or more sets of the at least one of the sets of feature tags, each feature tag in the set is mismatched from every other feature tag in the set by 1 to w nucleotides, wherein w is an integer from 2 to (y−4)÷2, wherein y is the number of nucleotides in the feature tags in the set, wherein the expression (y−4)÷2 is rounded up.

In some forms, the sequence-controlled storage object further includes a plurality of different digit tags, wherein the digit tags are present at the surface of the storage object, wherein the digit tags are number encoded.

In some forms, the sequence-controlled storage object further includes a plurality of different digit tags, wherein the digit tags are present at the surface of the storage object, wherein each of the plurality of different digit tags corresponds to the digit value of a different place in a multidigit number, wherein the number of different digit tags included in the storage object equals the number of places in the multidigit number. In some forms, each of the plurality of different digit tags is a member of a different set of digit tags, wherein each set of digit tags corresponds to a different place in the multidigit number. In some forms, each set of digit tags has a digit tag corresponding to each of the possible digit values of the place in the multidigit number to which the set of digit tags corresponds. In some forms, each of the different digit tags is hybridizably distinguishable from all of the other different digit tags in all of the sets of digit tags, wherein each of the different digit tags is hybridizably distinguishable from all of the different feature tags.

In some forms, the sequence-controlled storage object includes (a) one or more different sequence-controlled polymers, and (b) a plurality of different digit tags. In some forms, the digit tags are present at the surface of the storage object. In some forms, each of the plurality of different digit tags corresponds to the digit value of a different place in a multidigit number, wherein the number of different digit tags included in the storage object equals the number of places in the multidigit number. In some forms, each of the plurality of different digit tags is a member of a different set of digit tags, wherein each set of digit tags corresponds to a different place in the multidigit number. In some forms, each set of digit tags has a digit tag corresponding to each of the possible digit values of the place in the multidigit number to which the set of digit tags corresponds. In some forms, each of the different digit tags is hybridizably distinguishable from all of the other different digit tags in all of the sets of digit tags.

In some forms, the multidigit number corresponds to a feature attributable to one or more of the different sequence-controlled polymers. In some forms, the feature attributable to one or more of the different sequence-controlled polymers is a member of a set of related features, wherein each of the members of the set of related features has or can be associated with a different numerical value, wherein the different numerical values corresponds to the level or intensity of a given feature relative to the other features in the set of related features, wherein the multidigit number is equal to, proportional to, or the same as a given number of digits of the numerical value of the feature attributable to one or more of the different sequence-controlled polymers.

In some forms, the difference in the numerical values with which members of the set of related features have or can be associated are proportional to the similarity of the features in the set of related features. In some forms, the multidigit number is arbitrarily assigned to the feature attributable to one or more of the different sequence-controlled polymers to which the multidigit number corresponds. In some forms, the multidigit number is the same as a given number of digits of the numerical value of the feature attributable to one or more of the different sequence-controlled polymers starting from the most significant digit of the numerical value.

In some forms, each set of digit tags has the same number of members as the mathematical base in which the multidigit number is expressed. In some forms, the sequence-controlled storage object further includes one or more encapsulating agents, wherein the encapsulating agent coats or encapsulates the sequence-controlled polymers, wherein the encapsulating reagent can be reversibly removed through chemical or mechanical treatment.

In some forms, the feature tags are included in one or more of the encapsulating agents. In some forms, the one or more encapsulating agents are selected from natural polymers and synthetic polymers, or combinations thereof. In some forms, one or more encapsulating agents are selected from proteins, polysaccharides, lipids, nucleic acids, inorganic coordination polymers, metal-organic frameworks, covalent organic frameworks, inorganic coordination cages, covalent organic coordination cages, elastomers, thermoplasts, synthetic fibers, or any derivatives thereof.

In some forms, at least one of the sequence-controlled polymers is a single stranded nucleic acid, wherein the nucleic acid is folded into a three-dimensional polyhedral nanostructure including two nucleic acid helices that are joined by either anti-parallel or parallel crossovers spanning each edge of the structure, wherein the three-dimensional polyhedral structure is formed from single stranded nucleic acid staple sequences hybridized to the single stranded nucleic acid including bit-stream data, wherein the single stranded nucleic acid including bit-stream data is routed through the Eulerian cycle of the network defined by the vertices and lines of the polyhedral structure, wherein the nanostructure includes at least one edge including a double stranded or single-stranded crossover, wherein the location of the double strand crossover is determined by the spanning tree of the polyhedral structure, wherein the staple sequences are hybridized to the vertices, edges and double strand crossovers of the single stranded nucleic acid including bit-stream data to define the shape of the nanostructure, and wherein one or more of the staple sequences includes one or more feature tag sequences.

In some forms, a staple strand includes from 14 to 1,000 nucleotides, inclusive. In some forms, the single-stranded nucleic acid includes approximately 100 to 1,000,000 nucleotides, inclusive. In some forms, one or more staple strands include one or more feature tag sequences at the 5′ end, at the 3′ end, or at both the 5′ end and at the 3′ end. In some forms, the one or more feature tag sequences include one or more overhang oligonucleotide sequences. In some forms, the one or more feature tag sequences include oligonucleotide sequences complementary to one or more feature tag sequences attached to a different sequence-controlled storage object. In some forms, the sequence-controlled storage object further includes one or more additional sequence-controlled storage objects bound thereto.

Also disclosed are methods of storing desired sequence-controlled polymers as a sequence-controlled storage object, including
(a) assembling a sequence-controlled storage object from
(i) one or more different sequence-controlled polymers, and
(ii) a plurality of different feature tags, and
(iii) optionally one or more encapsulating agents,
wherein the feature tags are present at the surface of the sequence-controlled storage object,
wherein each different feature tag corresponds to a single feature attributable to one or more of the different sequence-controlled polymers,
wherein the single feature to which each different feature tag corresponds is a feature attributable to one or more different of the sequence-controlled polymers,
wherein the plurality of different feature tags collectively corresponds to a plurality of features that are collectively attributable to the plurality of different sequence-controlled polymers,
wherein each of the different feature tags is hybridizably distinguishable from all of the other different feature tags; and

(b) storing the sequence-controlled storage object.

In some forms, the method further includes the step of

(c) retrieving the desired sequence-controlled polymers. In some forms, retrieving the desired sequence-controlled polymers in step (c) includes isolating one or more sequence-controlled storage objects from a pool of sequence-controlled storage objects. In some forms, selection is determined by the sequence of one or more feature tags on the sequence-controlled storage object, the shape of the sequence-controlled storage object, affinity to a functionalized group bound to the sequence-controlled storage object, or combinations thereof.

In some forms, the method further includes the step of modifying the isolated sequence-controlled storage object by addition of one or more different feature tags. In some forms, addition of one or more different feature tags includes refolding, or re-organizing the sequence-controlled storage object with one or oligonucleotides including the different feature tags. In some forms, one or more sequence-controlled storage objects are isolated from a pool of sequence-controlled storage objects using Boolean logic. In some forms, Boolean NOT logic is used to delete one or more sequence-controlled storage objects from an object pool.

In some forms, the method further includes the step of

(d) accessing the desired sequence-controlled polymers. In some forms, storing the sequence-controlled storage object in step (b) further includes one or more of dehydrating, lyophilizing, or freezing the sequence-controlled storage object. In some forms, storing the sequence-controlled storage object in step (b) further includes one or more of rehydrating or thawing the sequence-controlled storage object for processing.

In some forms, storing the sequence-controlled storage objects includes storage in a matrix selected from cellulose, paper, microfluidics, bulk 3D solution, on surfaces using electrical forces, on surfaces using magnetic forces, encapsulated in inorganic or organic salts, and combinations thereof. In some forms, storing the sequence-controlled storage object in step (b) further includes digitally processing droplets containing sequence-controlled storage objects.

Also disclosed are methods of automating the assembly of a sequence-controlled storage object including using a device with flow, the device including

(a) means for flowing in the constituent components of the sequence-controlled storage object,

(b) means for mixing the constituent components,

wherein the means for mixing is operatively connected to the means for flowing,

(c) means for annealing the constituent components to form an assembled sequence-controlled storage object,

wherein the means for annealing is operatively connected to the means for mixing, and

(d) means for purifying the assembled sequence-controlled storage object,

wherein the means for purifying is operatively connected to the means for annealing.

In some forms, the method further includes

(e) means for introducing encapsulating agents to store the sequence-controlled object,

(f) means for introducing a plurality of feature tags attributable to the sequence-controlled polymer,

(g) means for selecting encapsulated sequence-controlled objects from an object pool, wherein the means of selection can be performed using Boolean logic, and

(h) means for removing the encapsulating agent to retrieve the sequence-controlled storage object.

In some forms, storage blocks are formed by encapsulating one or more sequence-controlled polymers within one or more encapsulating agents. Exemplary encapsulating agents include proteins, lipids, saccharides, polysaccharides, nucleic acids, and any derivatives thereof, as well as hydrogel and synthetic polymers including polystyrene, or silica, glass, and paramagnetic materials. These encapsulated biopolymers form discrete storage units that allow for controlled segregation of blocks. In some embodiments, storage blocks include sequence-controlled biopolymers folded into a specific nano-structured form, such as a nucleic acid nanostructure. In some forms, a storage block includes one or more discrete units within more than one type of sequence-controlled biopolymer. For example, in some forms, a nucleic acid sequence that is folded into a nucleic acid nanostructure, which contains or is associated with one or more polypeptides or other sequence-controlled biopolymers. In some forms, a storage block includes a nucleic acid sequence, encapsulated together with one or more polypeptides or other sequence-controlled biopolymers.

In some forms, the storage object can include a nucleic acid “scaffold” sequence that is folded into a nucleic acid nanostructure. The nucleic acid scaffold sequences can be of any length, for example, from 100-1,000,000 nucleotides. Typically, nucleic acid scaffold sequences are between 300-500,000 nucleotides, for example, from about 300 nucleotides to about 51,000 nucleotides in length, inclusive. In some forms, the methods provide the sequences of short single-stranded oligonucleotides staple strands of approximately 14-1,000 nucleotides in length, for example, approximately 14-600 nucleotides, which fold a single-stranded nucleic acid scaffold sequence into a nucleic acid nanostructure (e.g., polyhedron or DNA brick) having user-defined arbitrary geometries. Typically, the assembly of a nucleic acid nanostructure includes scaffold routing, staple strand selection, geometry and scaffold sequence inputs, oligonucleotide synthesis, and folding (“nano-structuring”), as performed with either scaffolded nucleic acid origami or non-scaffolded nucleic acid origami. The staple strands have nicks as part of the formation of the nanostructure, where the 5′ end of the staple meets the 3′ end of itself or another staple. These nicks can then have single-stranded overhang nucleic acid sequences of arbitrary sequence (“tags”).

The methods also provide nucleic acid encapsulation for storage, with nucleic acids being encapsulated within a layer of natural, or synthetic material. A nucleic acid of any arbitrary form can be encapsulated, for example, a linear, a single-stranded, base-paired double stranded, or a scaffolded nucleic acid. Exemplary encapsulating agents include proteins, lipids, saccharides, polysaccharides, nucleic acids, and any derivatives thereof, as well as hydrogel and synthetic polymers including polystyrene, or silica, glass, and paramagnetic materials. These encapsulated nucleic acids form discrete storage units that allow for controlled segregation of blocks.

Therefore, methods for creating Sequence-controlled polymer Storage objects (“SSOs”) are provided. In some forms, the storage objects are nucleic acid nanostructures or nucleic acid encapsulated units that represent Nucleic acid Storage objects (“NSOs”). The SSO storage “blocks” can be of variable size, are reconfigurable based on extrinsic cues, including buffer changes, enzymes, nucleic acid “keys,” temperature, electrical signals or light, and present identity tags for physical identification and retrieval or selection. The methods include assembling SSOs together into larger supra-storage blocks for spatially associating SSOs for segregation and associative storage applications. The methods also include functionalizing the staple strands to have tags that can be used for capture, rapid purification, and computation on SSOs. The methods provide sequence-controlled polymers as physical, structured units having arbitrary geometry and size that can be used to form supramolecular storage blocks. Nano-structuring, or encapsulating the storage blocks allows for a natural extension to spatial segregation of objects based on input signals, associating related sequence-controlled polymers into supra-block storage. The address space is multiplied by the number of tags in use, so 4^{(k*n)} where n is the number of nucleotides of the address per tag and k is the number of tags.

Selection and access of sequence-controlled polymers can be achieved by capture of SSOs mediated by specific and orthogonal interaction of the single-strand overhang tags. Overhang tags available in primer libraries known in the art can be included (Xu, et al., PNAS., V.106, (7) pp. 2289-2294 (2009)).

Tags from functionalized staple strands can be modified with a new addressing system, and the sequence-controlled polymer can be refolded with the new set of tagged staples, and/or overhang sequences. This allows for a dynamic addressing system that does not require re-synthesis of all the sequence-controlled polymer sequence. Sequence-controlled polymers encapsulated in silica or paramagnetic or sequence-controlled polymer-based nanoparticles can similarly be re-used, with display tags covalently or non-covalently attached through standard chemistries, specifying the number and stoichiometric ratios of specific overhang sequences. Methods for accessing sequence-controlled polymers, or subsets of sequence-controlled polymers from a pool of discrete SSOs are also provided. In some forms, accessing sequence-controlled polymers is carried out to enable selection via Boolean logic. For example, Boolean NOT logic can be used to delete sequence-controlled polymers from a sequence-controlled polymer pool. In some forms deleted sequence-controlled polymers are replaced, for example, with a new structure and set of addresses. In other forms, deleted sequence-controlled polymers are omitted from future computations/selections.

In some forms, the methods also optionally include long-term storage of SSOs. For example, the methods can include storage of scaffolded nucleic acid, or encapsulated nucleic acid for up to one year, up to one decade, up to two decades, three decades, or more than three decades. Typically, the methods do not include steps or processes detrimental to the stability and long-term storage of SSOs. For example, only selected outputs are processed by either PCR or sequencing. There are no required additions of new buffers and biological materials that can degrade the data. In some forms, DNA is stored in dry state to maximize its lifetime. When DNA is stored in dry state, appropriate mechanisms and systems can be used to segregate, order store and rehydrate the dry SSOs, for example, lyophilization and/or freezing of NSOs. In some forms, paper-based storage is used. Paper-based storage offers segregation of numerous nucleic acid storage solutions, or compartments that can be hydrated for selection and sequencing only when needed for storage retrieval. In further forms, systems include digital droplet-based microfluidics, for example, on electromagnetically actuated surfaces or in solution. Digital droplet-based microfluidics offer practical means of performing the wet biochemistry needed for the selection and retrieval steps. Therefore, in some forms, the methods include the use of digital droplet-based microfluidics for performing selection and retrieval steps.

In some forms, the storage objects are scaffolded nucleic acid nanostructures having a desired polygon or polyhedral shape. Therefore, in some forms, the methods include providing a nucleic acid sequence; creating a nucleic acid nanostructure, or a nucleic acid encapsulation unit that contains the sequence; and storing the nucleic acid nanostructure, or a nucleic acid encapsulation unit that contains the sequence.

In some forms, the methods also optionally include organizing sequence-controlled polymers within storage objects, such as nucleic acid nanostructures, or nucleic acid encapsulation units. In some forms, the methods also optionally include accessing the sequence. In further forms, the methods include retrieving the sequence from the storage object.

In some forms, the nucleic acid storage objects include a scaffold single-stranded nucleic acid of arbitrary length that is folded around the entire structure. Theoretically there is no limit to the size of the nucleic acid scaffold strand that is folded around the entire structure, however, in practical terms, the single-stranded nucleic acid scaffold typically includes between about 100 and 1,000,000 nucleotides. In some forms, the nanostructures also include one or more staple strands including one or more overhang oligonucleotide sequences. The staple strands are custom-designed to anneal to the scaffold strand to form any desired three dimensional nanostructure containing the sequence-controlled polymers. In some forms, the one or more overhang oligonucleotide sequences are feature tags. Exemplary feature tags include barcode sequences of approximately 4 to at least 30 nucleotides in length (Xu, et al., PNAS., V.106, (7) pp. 2289-2294 (2009)). In some forms the nucleic acid nanostructure has a geometric shape of a regular or irregular wireframe polyhedron. Typically, the geometric shape offers accessibility to the internal storage blocks by nucleic acids and enzymes. Therefore, in some forms the shape of the structure enables selection, or retrieval, or reconfiguration of the storage block, for example, due to porosity of the overall supra-molecular storage structure. Therefore, in certain forms, the desired target structure is one that offers diffusion of small molecules throughout it, for example, to provide access to enzymes and/or other molecules, such as nucleic acids. In other forms, the desired target structure prevents access of enzymes and/or other molecules, such as nucleic acids. In some forms, the SSO includes a hydrogel, polymer, glass, silica, or paramagnetic nanoparticle with specific overhang nucleic acid sequence or other high affinity and specificity tags that offer programmable interactions between distinct storage blocks in SSOs. Therefore, in some forms, the shape of the structure itself can be used as a means to select different or similar functionalities amongst SSOs.

Sequence-controlled biopolymer storage objects including nucleic acids or other sequence-controlled biopolymers encapsulated within natural or synthetic material are also provided. In some forms, a nucleic acid or other biopolymer of any arbitrary form can be encapsulated. For example, in some forms a linear, a single-stranded, a base-paired double stranded, or a scaffolded nucleic acid is encapsulated. Exemplary encapsulating agents include proteins, lipids, saccharides, polysaccharides, nucleic acids, synthetic polymers, hydrogel polymers, silica, paramagnetic materials, and metals, as well as any derivatives thereof. These encapsulated nucleic acids or other biopolymer are associated with one or more overhang nucleic acid sequences that are used for adding addresses, and/or purification tags. In some forms, multiple layers of encapsulation and overhang nucleic acids are designed for additional sorting and tagging the nature of the sequence-controlled polymers.

In some forms, the storage object has the geometric shape of a compact brick-like user-defined structure that can also stack end-to-end into long ribbons or into extended 2D or 3D crystalline-like arrays via either non-specific or specific stacking interactions that are controlled using buffer or nucleic acid overhangs or other physical association. In some forms, the one or more staple strands include “overhang” oligonucleotide sequences that are complementary to one or more staple strands from a different storage object, such as a different nucleic acid nanostructure, or to a bridging oligonucleotide. In some forms, one or more storage objects are organized into superstructures via complementarity of the nucleotide sequences from the one or more staple strands, or to the bridging nucleotide. For example, in some forms, nucleic acid nanostructures are organized into superstructures via complementarity of the nucleotide sequences from the one or more staple strands, or to the bridging nucleotide. In some forms, storage objects such as nucleic acid nanostructures or encapsulated nucleic acids are organized into superstructures based on user-defined associations between the storage blocks, noted above. The super-structured sequence-controlled polymers can then be specifically manipulated by external signals including pH, temperature, salts, nucleic acids, enzymes, light, etc. as well as microfluidic operations that may be droplet-based on-chip using electro-wetting or traditional 2-phase flow-based microfluidics. Application of mixing and splitting operations on selective pools of SSOs as well as other beads or reagents including cutting enzymes such as Cas9 or restriction enzymes offers ability to perform both complex and selective computation as well as storage manipulation and retrieval.

### BRIEF DESCRIPTION OF THE DRAWINGS

FIGS. 1A-1C are schematic representations of the objects described here, each showing different forms of diversity that can be generated within a pool of addressed storage objects. FIG. 1A depicts diversity in the size over several orders of magnitude of nanostructured storage objects that each have equivalent morphology (depicted as a closed cube), but which include between 0.5 kb to 100 kb of data, respectively. FIG. 1B is a schematic depicting several storage objects, each having diversity in geometry, including open wireframe polyhedra and compact brick-like geometries. FIG. 1C is a schematic depicting several storage objects having diversity in the number and orientation of single-stranded nucleic acid overhangs that are presented outwards at pre-defined geometric positions as one of several means of specifically associating multiple storage blocks into larger scale assemblies that can be stable or reconfigured or accessed in response to extrinsic cues.

FIG. 2 is a schematic chart depicting the associative nanostructured data framework amongst a pool of biopolymer storage objects. Generalized storage objects, shown as cubes, (A-D) can be maintained as separate, individual structures, or assembled into larger superstructures of AB, AC and D, respectively through a first signal event. The cuboid structures can reassemble and be re-sorted into differently-organized larger super-structures of ABC through a second signaling event and can be re-assorted to change geometries to expose internal blocks through a third signaling event, respectively, which may also be actuated extrinsically/externally through microfluidic or other mixing mediated by fluidics or solid-state manipulation of sub-pools of SSOs.

FIGS. 3A-3D are schematic diagrams, each depicted a step in the method to assemble a pool of nucleic acid storage objects. The scaffold strand of a nucleic acid origami object may be synthesized using template-free DNA synthesis using, for example, TDT polymerase, solid-state DNA synthesis, bacterial synthesis, PCR-based enzymatic synthesis, or another approach, multiply addressed with metadata tag overhang sequences on the staple strands (FIG. 3A); the scaffold strand including two feature tags (*) at each end of the scaffold, and the staple strands where overhang tags are used to encode multiple addresses (A and B) to the folded data are synthesized (FIG. 3B); the single-strand nucleic acid storage scaffold is combined with the staple oligonucleotides to fold into a DNA origami object (FIG. 3C); and adding the folded, multiply addressed DNA origami object to a storage pool (FIG. 3D).

FIGS. 4A-4D are schematic illustrations of encapsulated sequence-controlled biopolymers of any arbitrary forms into discrete SSOs for sequence-controlled polymer storage. FIG. 4A depicts single- or double-stranded DNA, RNA, PNA, LNA, or other nucleic acids or peptides or other sequence-controlled polymer (2), either with known/characterized errors in polymer sequence, or high-fidelity sequence. The sequence-controlled polymers, such as nucleic acids, are “packaged,” “encapsulated,” “enveloped,” or “encased” (4) in gel-based beads, protein viral packages (e.g., M13, adeno-associated virus, etc.), micelles, mineralized structures, siliconized structures, metals, paramagnetic materials, or designed polymers (6) that enclose or include one nucleic acids for multiplexed polymer storage using diverse polymers and polymer types (FIG. 4B) or more than one nucleic acid object (2, and 3) (FIG. 4C). These packaged nucleic acids (10) have molecular identifiers such as single-stranded tag sequences, or any purification tags (8) to allow specific sequence-controlled polymer selection and/or retrieval using Boolean logic (FIG. 4D). FIG. 4E is a schematic illustration showing the workflow of multiplexed attachment and encapsulation of sequence-controlled polymers (14), and modification of the molecular core (12) for downstream molecular logic operations and sequence-controlled polymer selection. Multiple sequence-controlled polymers are attached or absorbed by a molecular core. The molecular core is then functionalized with addressing/specificity tags (16) for multiplexed computation and selection.

FIGS. 5A-5E are schematic illustrations of methods to superstructure nucleic acid storage objects (NSOs) to spatially segregate and associate storage blocks. Blocks can be associated by direct complementarity of their tag sequences (FIG. 5A), or by a “bridge” DNA oligonucleotide complementary to two tags (FIG. 5B), or by kissing loop (FIG. 5C), or other secondary structure interactions, including base pair end-stacking into associative storage block super-structure (FIG. 5D). The associative storage block super-structure can then be used for further selection, dissociation of the individual NSOs, or re-assortment of the sequence-controlled polymers into different superstructures (FIG. 5E).

FIG. 6 is a schematic illustration providing a general overview of methods used to retrieve specific NSOs using complementary single-strand DNA sequences to the tags of the specified block(s). An exemplary method of NSO purification and selection is based on stationary phase complementary strands to tag(s) on the NSO: a single NSO is captured from a pool of NSOs captured using a capture support with sequences complementary to a (a′), and; captured NSOs having overhang sequence a are then released from the support. Tetrahedra are representative of any NSOs including encapsulated nucleic acids.

FIGS. 7A-7D are schematic illustrations depicting selection of the NSO based on both sequence and geometry placement of the overhang. FIGS. 7A and 7B depict tetrahedral NSOs displaying a and b tags on specific edges; FIG. 7C depicts a complementary geometric DNA nanostructure on a capture support, displaying a′ and b′ at positions to capture NSOs with a and b tags at appropriate geometric locations; FIG. 7D depicts a NSO with complementary a and b tags displayed at specific edges are selected by the larger DNA nanostructures. In this way, a NSO is specifically selected based not just on sequence of the overhang tags, but also on the geometry of the NSO. Tetrahedra are representative of any storage objects, including encapsulated nucleic acids or other biological or synthetic polymers.

FIG. 8 is a schematic illustration depicting the workflow for the method used to compute an AND logic operation on the NSO pool. A pool of differently addressed NSOs is depicted; a support (●) with a tag complementary to a (a′) is used to capture NSOs with overhang sequence a, resulting in a pool of NSOs having two different configurations of feature tags (a, b; and a, c, respectively) captured NSOs having overhang sequence a are then released from the support; a support with a tag complementary to b (b′) is used to capture NSOs further having overhang sequence b, released from the support; captured NSOs having overhang sequence b are then released from the support. Overall, this yields NSOs with overhang sequences a AND b by two-step capture purification. Tetrahedra are representative of any storage objects, including encapsulated nucleic acids or other biological or synthetic polymers.

FIG. 9 is a schematic illustration depicting the workflow for the method used to compute an OR logic operation on the NSO pool. A pool of differently addressed NSOs is depicted; NSOs containing an overhang of sequence a OR an overhang of sequence e are captured using capture support (●) with sequences complementary to a (a′) and e (e′), with NSOs containing neither being washed off the capture support; captured NSOs having an overhang of sequence a OR an overhang of sequence e are then released from the support. Tetrahedra are representative of any storage objects, including encapsulated nucleic acids or other biological or synthetic polymers.

FIG. 10 is a schematic illustration depicting the workflow for the method used to compute a NOT logic operation on the NSO pool. A pool of differently addressed NSOs is depicted; NSOs having overhang tag sequences of a are captured on the capture support (●) using the capture sequence complementary to a (a′) and thus unbound objects from this capture support are all those objects which do not contain the a overhang, thus NOT a. Tetrahedra are representative of any storage objects, including encapsulated nucleic acids or other biological or synthetic polymers.

FIG. 11 is a schematic illustration depicting the workflow for the methods used to read out the selected NSO(s). Desired NSOs are first selected; NSOs are denatured, and the released single-strand nucleic acid scaffold is amplified by virtue of master primer sequences flanking the DNA sequence; and the scaffold strand is sequenced. Alternatively, mass spectrometry or other analytical procedure may be used that does not require direct polymer-based sequencing to decode the sequence-controlled polymers, based on mass, charge, length, or other physicochemical properties. Tetrahedra are representative of any storage objects, including encapsulated nucleic acids or other biological or synthetic polymers.

FIG. 12 is a schematic illustration depicting the workflow implemented within an exemplary microfluidic device allowing for the automated assembly and purification of a NSO. The scaffold and staples are offered as inputs to a mixing chamber (“mixer”), followed by an annealing chamber (annealer), followed by a dialysis or filtering chamber for purification of the NSO from staples (exchanger). In cases where a sequence-controlled polymer, or other materials are used for storage encapsulation in particulate form, other upstream preparative devices may be interfaced, and bypass the need for annealing, for example.

FIG. 13 is a schematic illustration depicting the workflow implemented within an exemplary microfluidic device allowing for the rapid purification of the nanostructure NSOs, including the ability to “daisy-chain” the devices for complex logic gating. Multiple out-ports on the capture chamber allow for AND/OR/NOT logic implementation at the microfluidic level. A storage pool of NSOs; exemplary signal input for selection of the target NSOs based on their tag overhangs; an exemplary capture chamber for capturing, washing, and elution for selecting based on the input signal(s); unlimited number of signal input and capture chambers for executing the selection; further exemplary signal input for selection of the target NSOs based on their tag overhangs; further exemplary capture chamber for capturing, washing, and elution for selecting based on the input signal(s); the final output where the scaffold sequence is amplified, sequences and decoded. Electro-wetting-based droplet manipulation devices such as the Mondrian may be used to perform these controlled mixing and splitting operations in a rapid and controlled manner that is also fully automated.

FIG. 14 is a schematic chart depicting the elements of an exemplary system for creating, storing and organizing sequence-controlled polymers as re-useable “storage blocks” or computational molecular elements. A structured storage block, such as a cubeoctahedron is shown as a square structured nucleic acid storage block. The storage blocks can be of many sizes, from small to as large, as needed to accommodate sequence-controlled polymers. Each block can have multiple different file handles, or indices (depicted as a-d), allowing for multiple addressing of sequence-controlled polymers for selections and operations. Specific modifications, such as overhang sequences, can be used to associate multiple blocks together into large superblocks of storage, for rapid retrieval, re-assortment and computation with associated or categorized sequence-controlled polymers. Modified overhangs also allow for use of Boolean logic AND, OR, and NOT operations on the storage blocks, for example, to select for purification of one or more storage blocks from a pool of storage blocks.

FIGS. 15A and 15B are flow charts. FIG. 15A demonstrates the work flow within one system for long-term storage of sequence-controlled polymers in the form of storage blocks of DNA. Any number of nucleic acid storage objects (e.g., 1-10's of millions) are blotted and freeze-dried to a long-term storage material (“paper”) for segregation of sequence-controlled polymers and for later retrieval. Dried storage blocks are selectively rehydrated by addition to blot with water or buffer. The process can be automated to selectively pull out the right spatially segregated storage pool, with the hydrated storage blocks being processed as described, and sequenced, for example by handheld devices, or bench-top sequencers. FIG. 15B is a flow chart describing the general approach towards molecular data storage and computation. Any digital files and folders from a computer. The digital files are encoded and/or converted to a molecular storage code (e.g., nucleotides, amino acids, polymers, atoms, surfaces. The code is written to the physical storage block used to store the data. The stored data is associated with a set of address codes to identify the storage block. The addresses are affixed to the storage block such that they can be used for subsequent reading, manipulation, selection, and computation, including physical tags, electrostatic or magnetic properties, chemical properties, or optical properties. The storage blocks with addresses are placed in a pool of other storage blocks for storage and computation. The pool is separated based on the physical properties, with some storage blocks satisfying the selection criteria and others not and are sorted as such. Many cycles of this and other selection criteria can take place in parallel or in series. The sorted storage block(s) of interest are purified from the pool. The sorted storage block(s) are read out and decoded to digital format. The original digital file is retrieved from the pool.

FIG. 16 is a line graph showing % Readable Message Population over Time. Degradation of NSOs is initiated at the point (▴) upon exposure to external switches such as the presence of light, heat, enzymes, chemical reactants, or air, to activate the timed degradation of the DNA, RNA, or other nucleic acid, resulting in a degraded message pool.

FIGS. 17A-17D are schematic illustrations of the silica encapsulation of sequence-controlled polymer storage blocks. FIG. 17A depicts a silica particle (18). FIG. 17B depicts the silica particle, modified (20) to allow adsorption of DNA particles. FIG. 17C depicts nucleic acid storage blocks (22) adsorbed to the surface-modified silica particles. FIG. 17D depicts a secondary silica shell (24) that is grown on the silica with the nucleic acid storage blocks adsorbed (26). This shell provides environmental protection for the nucleic acid storage blocks. FIG. 17E is a schematic of an exemplary DNA assembly (a double-crossover or DX tile) containing Cy3 and Cy5 energy transfer pair as a readout for monitoring the structure of the DX tile. FIG. 17F is a graph showing Intensity (cps) over Wavelength (nm) corresponding to the emission spectra of the DX tile prior to the encapsulation process (−), and the emission spectra of the DX tile upon completion of the encapsulation step (--), respectively.

FIGS. 18A-18F shows example outcomes from NSO super-structuring. FIG. 18A depicts a single (monomer) NSO. FIGS. 18B-D each depict an exemplary “dimer” of two NSOs brought together at their vertices (FIG. 18B), along their edges (FIG. 18C), or at their faces (FIG. 18D), respectively, using overhang addressing. FIGS. 18E-18F each depict a “tetrahedra” of NSOs coming together in larger superstructures, as an extended tetramer addressed to come together along the edges via complementarity (FIG. 18E), and with different addresses, allowing assembly of a more compact configuration (FIG. 18F), respectively.

FIGS. 19A-19C are schematic illustrations depicting the molecular shelling of the storage objects. FIG. 19A is a scheme depicting the loading of a porous core (28) with multiple sequence-controlled polymers (30), shelling (32) and appending of feature tags to the shelled storage object (36). FIG. 19B is a scheme depicting the first stage in assembly of a storage object (44) from a core (38), to which recognition sites (40) are first bound, then sequence-controlled polymers (42) including one or more tags specific to the recognition sites bound to the core are complexed. FIG. 19C is a scheme depicting the final step of the assembly of the storage object (50) depicted in FIG. 19B. The core (44) and associated sequence-controlled polymers, are then encapsulated in a shell (46), to which the feature tags (48) are then bound.

FIGS. 20A-20B are schematic illustrations depicting the molecular shelling of the storage objects including multiple sequence-controlled polymers and modification of the shell with affinity tags for multiplexed molecular logic operations and sequence-controlled polymer selection. Sequence-controlled polymers (54) that are (FIG. 20A) attached to a molecular core (52) are further surrounded by a molecular shell (56) and functionalized with addressing/specificity tags (58) for multiplexed computation (60); or (FIG. 20B) sequence-controlled polymers (64) that are absorbed by a molecular core (62) are further surrounded by a molecular shell (68) and functionalized with addressing/specificity tags (66) for multiplexed computation (70). The shell or core has a readout based on optical, magnetic, electric, or physical properties of the shell/core.

FIGS. 21A-21B are schematic illustrations depicting storage wherein sequence-controlled polymers are in the molecular core or shell. FIG. 21A depicts a storage object formed from sequence-controlled polymers on a molecular core, which has a readout based on optical, magnetic, electric, or physical properties of the core. The molecular core contains address/specificity tags for molecular logic and sequence-controlled polymer retrieval operations. FIG. 21B depicts a storage object formed from sequence-controlled polymers on a molecular shell surrounding a molecular core. The shell/core has readouts based on the optical, magnetic, electric, or physical properties of the shell/core. The shell is functionalized with addressing/specificity tags for molecular logic and sequence-controlled polymer retrieval operations.

FIG. 22 is a schematic diagram of the proposed workflow for biomolecule storage and retrieval using nucleic acids as an example. Biomolecules are extracted from samples of any origin and collected into microplates. Upon encapsulation and barcoding of samples, the capsules are pooled together. Samples are selected using probes that contain optical markers or chemical/biochemical affinity tags. The tags are used for optical or mechanical sorting of samples from the pool. The rest of the pool are returned for storage until further use.

FIGS. 23A-23B are schematics of data panels that demonstrate a proof-of-concept storage and retrieval of biomolecules using synthetic barcoded packets. Capsules that contain B. taurus (contains “Eukaryote”, “Animalia”, “2021-01-05”, and “Bos taurus” labels) and M. musculus (contains “Eukaryote”, “Animalia”, “2021-01-03”, and “Mus musculus” labels) genomes were targeted for retrieval from the pool that contains H. sapiens total RNA (contains “Eukaryote”, “Animalia”, “2021-01-03”, and “Homo sapiens” labels) and SARS-CoV-2 RNA genome (contains “Riboviria”, “Orthornavirae”, “2020-12-20”, and “SARS-CoV-2” labels). Boolean logical query using molecular probes that matches the query strings “Eukaryote”, “Animalia”, and “Homo sapiens” were added into the pool (FIG. 23A). Fluorescence gate selection using different colors associated with each probe identify the populations of interest. Selection of populations that are positive on “Eukaryote” AND “Animalia” selects B. taurus, M. musculus, and H. sapiens. Additional “Homo sapiens” gate can be used to select population that are negative for “Homo sapiens” or in Boolean logic representation, NOT Homo sapiens. Thus, the final Boolean logical search query is “Eukaryote” AND “Animalia” AND (NOT “Homo sapiens”), which selects B. taurus and M. musculus as validated using quantitative real-time polymerase chain reaction (FIG. 23B).

FIGS. 24A-24B demonstrate a proof-of-concept reaction using barcodes on sample surfaces as initiators. FIG. 24A is a schematic showing hybridization-based selection; Capsules that contain the “Homo sapiens” tag (labelled as “z” in the figure) is hybridized with complementary z* tag, which also includes a toehold sequence “a*” and stem sequence “b*”, triggering hybridization chain reaction (HCR) between two hairpin structure modified with a marker, which can be a dye or a chemical/biochemical tag. FIG. 24B is a graph of Intensity (a.u.) over wavelength (nm) for each of HCR modified, single probe modified and orthogonal barcode+HCR control capsules, respectively, showing fluorescence enhancement observed for HCR-amplified capsules, as compared to capsules that were hybridized with only a complementary strand containing a single dye.

FIGS. 25A-25C are drawings of an exemplar millifluidic device that can be used to encapsulate and barcode biomolecules using emulsion reactors. FIG. 25A is a CAD design of a millifluidic device. FIG. 25B shows a 3D printed millifluidic device. FIG. 25C is a schematic detailing the formation of a droplet within the device pictured in FIG. 25B, with 2 mM Ca2+ and 2% (w/w) low-viscosity alginate is flowed into a channel that is connected to T-junction where surfactant-containing oil is being flowed.

FIG. 26 is a schematic of the process of retrieving a collection of particles corresponding to a range of some numerical feature of the underlying biomolecule. Each possible digit value at each digit place of the number is associated with a distinct orthogonal barcode, permitting retrieval of ranges of values by selecting particles with particular digit values at a subset of the digit places. As an example, a numerical feature can be represented in base 3, and the collection of particles with barcodes corresponding to numbers in the range [1000, 1100] can be retrieved by selecting particles with the barcode associated with “1” in the 27 s place and “0” in the 9 s place.

FIG. 27 is a schematic of the barcode sequence design process that enables exact similarity-based retrieval with respect to a feature whose similarity metric is simple enough to permit an exact isometric embedding from feature similarity space to a low-dimensional hypercube. The isometric embedding corresponds directly to an assignment of barcodes to each particle that permit similarity-based retrieval. As an example, the schematic shows the nucleic acid sequence CCCATCGTGTCATTA (SEQ ID NO:1) having a selection of four mutations at different positions in the sequence, and a simple similarity metric represented in a cyclic graph with 8 nodes that may be isometrically embedded exactly into a 4-dimensional hypercube graph.

FIG. 28 is a schematic of the barcode sequence design process that enables approximate similarity-based retrieval with respect to a feature with an arbitrarily complex similarity metric. The feature similarity space is simplified using standard dimensional reduction to reduce it to a small number of dimensions. These dimensions are then approximated further by binning, after which they can be embedded directly into a hypercube graph whose nodes represent mutational variants of a set of barcodes. As a proof-of-concept example, the schematic shows the process beginning with a complex similarity metric derived from 4187 SARS-CoV2 genomes whose pairwise genetic similarity was computed. This similarity metric was reduced to 18 dimensions using multidimensional scaling (MDS); for visualization purposes only here, the number of dimensions was reduced further to 2 dimensions before plotting. After binning, linear regression showed a strong correlation between the original similarity metric and the final distance in a 54-dimensional hypercube embedding. The hypercube embedding corresponds directly to an assignment of 6 barcode sequences to each node in the original feature space, having 9 mutation sites each. Exemplary bar code sequences include GCCTTGTATGTGAATATCCGTGTCA (SEQ ID NO:2), and GGAGAATGATTAGCACGGAGAGTGG (SEQ ID NO:3).

### DETAILED DESCRIPTION OF THE INVENTION

Encapsulation chemistry is combined with the precision of DNA base-pairing as molecular barcodes for identification and retrieval of individual samples to realize a room-temperature ultradense storage and retrieval system for DNA, RNA, peptides, and proteins. The disclosed technology is broadly applicable to storage and cataloging biomolecules from any source, such as human patients, animals, and the environment.

In one implementation, biomolecules are surface-adsorbed on the surface of a capsule with a diameter in the range of 1 nm to 100 μm. Biomolecules are attached covalently or non-covalently on the surface of the particle. Encapsulation of the surface-adsorbed molecule proceeds by condensation, polymerization, and crosslinking of inorganic and organic monomers on the surface-adsorbed monomers. The surfaces of the encapsulated biomolecules are then labeled using single-stranded DNA barcodes.

In another implementation, biomolecules are encapsulated inside the channels of porous particles.

In another implementation, biomolecules and encapsulation reagents are introduced into wells in a microplate containing adsorbent particles using an automated liquid handling device.

In another implementation, biomolecules are trapped in emulsions using microfluidic channels controlled using electricity or photons and encapsulated within the emulsion. Barcodes are attached post-encapsulation.

In another implementation, biomolecules and barcodes are combined and encased in emulsions composed of multiple layers of aqueous and organic solvents using microfluidic approaches. Permanent encapsulation using organic or inorganic polymers and barcoding proceeds in one step.

In another implementation, molecular barcodes may include non-standard nucleotides or non-phosphate backbones to improve the stability of the barcodes.

In another implementation, molecular barcodes can be attached using chemical synthesis or enzymes.

Selection of encapsulated samples proceeds by hybridization of probes that are complementary to the barcodes of interest. Probes may contain optical, chemical, and biochemical markers for optical or mechanical sorting using millifluidic or microfluidic approaches.

In another implementation, chemical and biochemical reactions can be performed on the tags to increase sorting throughput.

The storage and retrieval system isolate the biomolecule of interest from the environment for protecting the integrity of the biomolecule over ten years or longer and eliminates the need for low-temperature storage conditions. Barcoding micron-to-nanoscale capsules enable the pooling of all samples in a single vessel rather than millions of individual tubes, thus reducing the footprint of biomolecular storage to size dimensions that can sit on top of a desktop.

Herein capsules are termed as particles containing the biomolecules the encapsulated molecules and are labeled with molecular barcodes for retrieval. The encapsulants herein can be composed of organic and inorganic materials. The molecular barcodes herein are short-primer strands of oligonucleotides derived from a pool of 240,000 [Xu, et al. Proceedings of the National Academy of Sciences 106, 2289-2294, doi:10.1073/pnas.0812506106 (2009)]. The barcodes are taken from this pool and used with or without sequence modification to permit retrieval of individual particles or collections of related particles. The choice of barcodes permits retrieval of collections of related particles that correspond to discrete categories, ranges of a discretized numerical feature (e.g., date of sample collection), or similarity-based retrieval with respect to a continuous or non-discrete feature. The encapsulation and barcoding approach can be performed using automated liquid handling equipment or millifluidic/microfluidic devices. Samples are selected for retrieval through the addition of probes that hybridize on target barcodes. Selected samples are sorted from solution using optical and mechanical sorting methods using, but not limited to, fluorescence-activated sorting, magnetic sorting, electrokinetic sorting, and similar sorting approaches. Selection and sorting of samples can also be performed using automated liquid handling equipment or millifluidic/microfluidic devices.

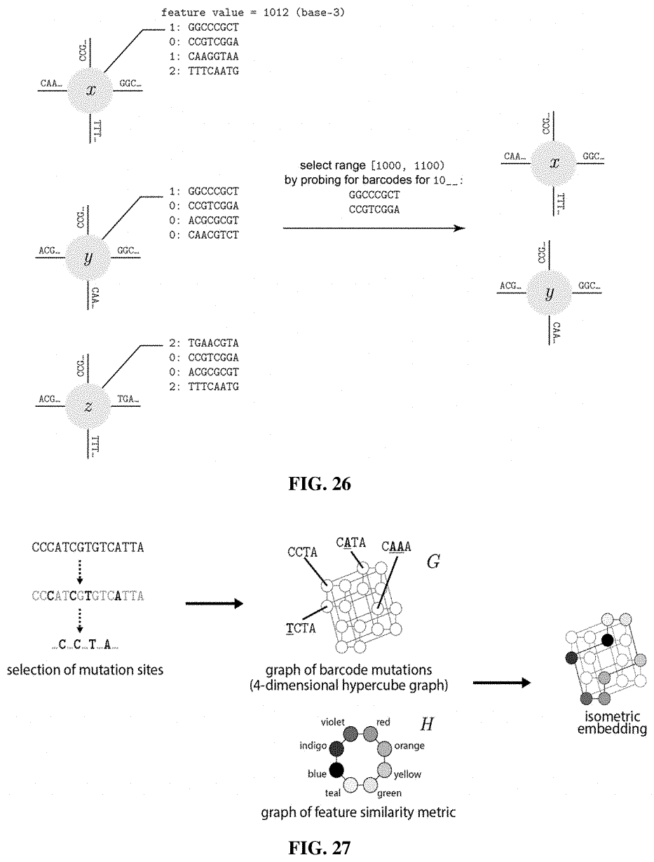

The various schemes by which barcodes may be assigned to particles in order to permit selection of different collections of related particles are described as follows. To permit retrieval of collections of particles belonging to one of a number of discrete categories, one orthogonal barcode sequence is associated with each category and a particle's membership in each category is indicated by the particle's corresponding selection of barcodes. To permit retrieval of collections of particles belonging to ranges of a discretized numerical feature, one orthogonal barcode sequence is associated with each possible digit value at each digit of the number. With this approach, a collection of particles corresponding to any numerical range of the feature may be retrieved, as long as this range can be specified by selecting for particular digit values at some subset of the digits in the number. An example of numerical range retrieval is shown in FIG. 26.

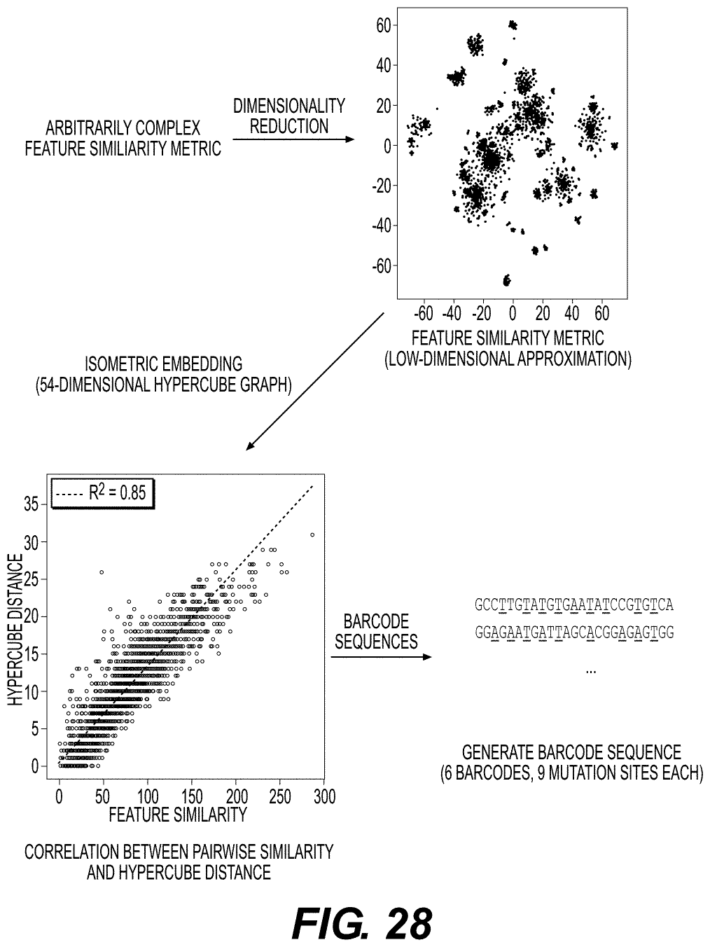

To permit retrieval of collections of particles that are similar to each other with respect to continuous or non-discrete features, barcode sequences are mutated at a small number of carefully selected sites within the sequence. A restricted set of mutated variant barcode sequences are represented in a graph G, such as, but not limited to, a hypercube graph. The mutation sites are selected so that the graph G faithfully represents the binding affinity between the barcodes and the complementary sequences to the barcodes that are to be used as probes. The similarity space of the continuous feature is also represented in a graph H, which is subsequently embedded isometrically into the graph G. For certain simple graphs H, an exact isometric embedding may be found using polynomial time algorithms. For arbitrary, complex graphs H, the isometric embedding may be found by first performing dimensional reduction on the corresponding metric space represented by H. The dimensional reduction may be performed using any standard technique that attempts to preserve distance during the transformation. The lower-dimensional space may then be discretized to approximate an isometric embedding into G. Examples of finding an isometric embedding both when H is simple and complex are shown in FIGS. 27 and 28.

### I. Definitions

A “feature tag” is an oligonucleotide of a defined sequence that corresponds to a feature attributable to a sequence-controlled polymer. Correspondence of a feature to a feature tag refers to a one-to-one mapping of that feature to that feature tag.

A “feature attributable to a sequence-controlled polymer” refers to a feature that the sequence-controlled polymer possesses or embodies.

“Hybridizably distinguishable” means orthogonal for hybridization.

“Similarity-encoded” means that the relative hybridizability of the feature tags is related to the similarity of the features to which the feature tags correspond, with feature tags corresponding to more similar features having closer relative hybridizability than feature tags corresponding to less similar features. In a similarity-encoded set of feature tags it is useful for the difference in the hybridization energy of the feature tags in the set to be a monotonically increasing function of the similarity of the features to which the feature tags correspond.

“Relative hybridizability” means the hybridization energy of a probe to an feature tag relative to the hybridization energy of the same probe to a different feature tag.

“Hybridization ordered” means that each of the feature tags in the set differs from all of the other feature tags in the set by 1 to x mismatched nucleotides, where the mismatched nucleotides are (i) at least two nucleotides from either end of the feature tag and (ii) are separated by at least one matching nucleotide in the feature tags, where x is the number of different nucleotide positions in the feature tags that are varied in the set.

“Number encoded” means that each different digit tag corresponds to the digit value of a different place in a multidigit number.

The term “payload” refers to the sequence-controlled polymers for storage. For example, in nucleic acid storage, the payload is the specified nucleotide sequence. The terms “desired polymer” or “desired nucleic acid” are used interchangeably to specify the payload that is contained in the sequence within a given storage object.

The term “sequence” refers to any natural or synthetic sequence-controlled polymer sequence to be stored. For example, when nucleic acid is used to store data, the “sequence” is the nucleic acid sequence of the nucleic acid. The nucleic acid can be in the form of a linear nucleic acid sequence, a two-dimensional nucleic acid object or a three-dimensional nucleic acid object. The nucleic acid can include a sequence that is synthesized, or naturally occurring. It can be considered that the sequence of any sequence-controlled polymer encodes the data represented by the sequence of the polymer. For example, a naturally occurring nucleic acid is a sequence-controlled polymer where the naturally occurring sequence of the nucleic acid is the data encoded by the nucleic acid.

The term “bit” is a contraction of “binary digit.” Commonly “bit” refers to a basic capacity of information in computing and telecommunications. A “bit” conventionally represents either 1 or 0 (one or zero) only, though other codes can be used with nucleic acids that contain 4 nucleotide possibilities (ATGC) at every position, and higher-order codecs including sequential 2-, 3-, 4-, etc. nucleotides can alternatively be employed to represent bits, letters, or words.

The terms “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs or modified nucleotides thereof, including, but not limited to locked nucleic acids (LNA) and peptide nucleic acids (PNA). An oligonucleotide is typically composed of a specific sequence of four nucleotide bases: adenine (A); cytosine (C); guanine (G); and thymine (T) (uracil (U) for thymine (T) when the polynucleotide is RNA). Thus, the term “oligonucleotide sequence” is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides.

The terms “staple strands” or “helper strands” are used interchangeably. When used in the context of a nucleic acid nanostructure object, “Staple strands” or “helper strands” refer to oligonucleotides that work as glue to hold the scaffold nucleic acid in its three-dimensional geometry.

The terms “scaffolded origami,” “origami” or “nucleic acid nanostructure” are used interchangeably. They can be one or more short single strands of nucleic acids (staple strands) (e.g., DNA) that fold a long, single strand of polynucleotide (scaffold strand) into desired shapes on the order of about 10 nm to a micron, or more. Alternatively, single-stranded synthetic nucleic acid can fold into an origami object without helper strands, for example, using parallel or paranemic crossover motifs. Alternatively, purely staple strands can form nucleic acid storage blocks of finite extent. The scaffolded origami or origami can be composed of deoxyribonucleotides (DNA) or ribonucleotides (RNA), or analogs or modified nucleotides thereof, including, but not limited to locked nucleic acids (LNA) and peptide nucleic acids (PNA). A scaffold or origami composed of DNA can be referred to as, for example a scaffolded DNA origami or DNA origami, etc. It will be appreciated that where compositions, methods, and systems herein are exemplified with DNA (e.g., DNA origami), other nucleic acid molecules can be substituted.

The terms “nucleic acid encapsulation,” and “nucleic acid packages” are used interchangeably. They refer to the method of encapsulating nucleic acid of any length or geometry by a material to form discrete units. The encapsulating material can be of any appropriate natural or synthetic material, for example, proteins, lipids, saccharide, polysaccharides, natural polymers, synthetic polymers, or derivatives thereof. The encapsulated units are therefore in the form of gel-based beads, protein viral packages, micelles, mineralized structures, siliconized structures, polymer packaging, or any combinations thereof.

The terms “sequence-controlled polymer” or “sequence-controlled macromolecule” refer to a macro-molecule that is composed of two or more distinct monomer units sequentially arranged in a specific, non-random manner, as a polymer “chain.” That is, a sequence-controlled polymer is a polymer where the order of the monomer units in the polymer is non-random, specified, or specifically determined. The arrangement of the two or more distinct monomer units constitutes a precise molecular “signature,” or “code” within the polymer chain. Sequence-controlled polymers can be biological polymers (i.e., biopolymers), or synthetic polymers. Exemplary sequence-controlled biopolymers include nucleic acids, polypeptides or proteins, linear or branched carbohydrate chains, or other sequence-controlled polymers. Exemplary sequence-controlled polymers are described in Lutz, et al., Science, 341, 1238149 (2013).

The term “sequence-controlled polymer object” refers to an object that includes a sequence-controlled polymer and one or more feature tags, digit tags, and/or barcodes.

The terms “sequence-controlled polymer storage object,” or “SSO,” or “storage block,” or “storage object” are used interchangeably. They refer to an object that includes a sequence-controlled polymer and one or more feature tags or barcodes. The polymer includes a discrete sequence, and the feature tags enable selection, organization, and isolation of the storage object. In some forms, storage objects include sequence in the form of a continuous stretch of sequence-controlled polymer. In some forms, storage objects include discontinuous segments of sequence. In some forms, storage objects include a sequence-controlled polymer that is folded into a two or three dimensional shape. For example, sequence-controlled polymers can be folded into a nanostructure form that is the entire SSO, such as a nanostructured nucleic acid object. In some forms, the sequence-controlled polymer is combined with one or more additional materials to form a nanoparticle. SSOs can take any arbitrary form, for example, a linear sequence molecule, or a two-dimensional object, or a three-dimensional object. Sometimes, the storage objects are made from scaffold polymer sequence with or without staple nucleic acid sequences, or from sequence-controlled polymers of any arbitrary length/form, encapsulated within one or more encapsulating agents.

The terms “Nucleic acid storage object,” or “NSO” are used interchangeably to refer to a SSO that includes nucleic acid as the sequence. An NSO includes one or more segments of nucleic acid sequence. In some forms, NSOs are in the form of a single-stranded nucleic acid scaffold that folds onto itself, or multiple single-stranded nucleic acid molecules that self-assemble into a programmed geometric block. NSOs can take any arbitrary form, for example, a linear nucleic acid sequence, a two-dimensional nucleic acid object or a three-dimensional nucleic acid object. Sometimes, the nucleic acid storage objects are nucleic acid objects made from scaffold nucleic acid with or without staple nucleic acid sequences, or from encapsulated nucleic acid of any arbitrary length/form, or any combinations thereof. The NSO can be composed of deoxyribonucleotides (DNA) or ribonucleotides (RNA), or analogs or modified nucleotides thereof, including, but not limited to locked nucleic acids (LNA) and peptide nucleic acids (PNA). An NSO composed of DNA can be referred to as a DNA storage object (“DMO”), etc. It will be appreciated that where compositions, methods, and systems herein are exemplified with DNA (e.g., DMOs), other nucleic acid molecules can be substituted.

The terms “splint strand” and “bridge strand” are used interchangeably to refer to a nucleic acid sequence that is complementary to two or more strands of nucleic acid sequences at distinct, non-overlapping locations. For example, a first region on a splint strand is complementary to a region on an overhang tag of a first NSO, whilst a second region on the same splint strand is complementary to a region of an overhang tag of a second NSO. The two regions of the splint strand are located so that the binding of the first NSO does not sterically hinder the binding of the second NSO. The splint or bridging strand therefore serves to bring the two NSOs into proximity with a fixed, predetermined distance.

The terms “feature tag,” “nucleic acid overhang,” “DNA overhang tag,” and “staple overhang tag” are used interchangeably to refer to nucleotides associated with SSOs that can be functionalized. In some instances, the overhang tag contains one or more nucleic acid sequences that encode metadata for the associated SSOs. In some forms, nucleotides are added to the staple strand of a NSO. In some forms, the overhang tag contains sequences designed to hybridize to other stationary-phase objects such as magnetic beads, surfaces, agarose or other polymer beads. In some instances, the overhang tag contains sequences designed to hybridize other nucleic acid sequences such as those on tags of other SSOs, or on splint strands. In other instances, the overhang contains one or more sites for conjugation to a molecule. For example, the overhang tag can be conjugated to a protein, or non-protein molecule, for example, to enable affinity-binding of the SSOs. Exemplary proteins for conjugating to overhang tags include biotin and antibodies, or antigen-binding fragments of antibodies. In some forms, overhang tags are designed and implemented within SSOs to enable programmable affinity and specificity between two interacting storage objects, whatever their implementation, for example, using since the principles of Boolean logic and computation.

The terms “encapsulating,” “enveloping,” “coating,” “covering,” and “shelling” are used interchangeably to refer to the process by which SSOs are completely or partially enclosed by an encapsulating agent. The term “encapsulating agent” refers to a molecular entity, such as a polymer or other matrix.

### II. Methods and Systems for Sequence-Based Storage

Sequence-controlled polymers, such as nucleic acid molecules (e.g., DNA), represent an excellent storage object and medium, having a very high potential for information density (e.g., up to 10²⁴ bits/kg for DNA), long-term stability, and low cost of energy to maintain.

Methods for the storage of sequence-controlled polymers formed into nanostructures have been developed. Sequence-controlled polymers are folded into, or embedded within well-defined, discrete structures that serve as sequence-controlled polymer storage objects (SSO). Therefore, distinct packages of sequence-controlled polymers are provided as three-dimensional structures with multiple faces that include one or more specific sequence tags. Through manipulation of SSO structures, the methods enable the partitioning, association, and re-assortment of polymer sequences within each SSO. Information retrieval is achieved rapidly by interpreting the sequence, structure or other physical or chemical property of the sequence-controlled polymer. Therefore, the methods enable rapid and efficient organization and access of sequence-controlled polymers stored within SSOs.

Methods for the storage of sequence-controlled polymers of any length, or any form have also been developed. Typically, sequence-controlled polymers having a sequence of any desired length are packaged, encapsulated, enveloped, or encased in gel-based beads, protein viral packages, micelles, mineralized structures, siliconized structures, or polymer packaging, herein referred to as “sequence-controlled polymer storage block.” In some forms, the synthetic polymers or biopolymers include a single, continuous polymer, contained within a nanoparticle. In some forms, the synthetic polymers or biopolymers include many such polymers that are combined within a single nanoparticle. These discrete biopolymer “packages” serve as Sequence-controlled polymer Storage objects (SSOs) and allow incorporation of one or more specific tags on the surface of the structures. Some exemplary tags include nucleic acid sequence tags, protein tags, carbohydrate tags, and any affinity tags.

In some forms, the sequence-controlled polymer is a biopolymer, such as a nucleic acid sequence, a polypeptide amino acid sequence, a protein, a carbohydrate sequence, or combinations thereof.

A. Sequence-Controlled Polymer Storage

Methods of storing polymers can include the assembly of sequence-controlled polymer storage objects (SSOs) including one or more polymer sequences and one or more feature tags. The one or more polymer sequences can be present either within the particle core, or associated with one or more layers surrounding the core, for example, embedded within an encapsulating material. The indices/affinity tags are exposed and accessible. For example, the indices/affinity tags are to embedded within or otherwise attached to the external surface of the particles. The manner in which the indices/barcodes are attached to the external surface of the core particle and/or sequence can be varied according to the desired manner for pooling, sorting, organizing and accessing the sequence-controlled polymers.

In some forms, the “shell” that is the product of “shelling” contains the sequence-controlled polymer.

1. Nucleic acid Nanostructures

In exemplary forms, the sequence-controlled biopolymer is a nucleic acid. Methods for the storage of sequence-controlled polymers using nucleic acid nanostructures have been developed. Nucleic acid nanostructures formed from single-stranded nucleic acid scaffolds of up to tens of kilobases (kb) are folded into well-defined, discrete structures that serve as nucleic acid storage objects (NSOs). Therefore, distinct packages of sequence-controlled polymers are provided as three-dimensional nucleic acid structures with multiple faces that include one or more specific sequence tags. Through manipulation of NSO structures, the methods enable the partitioning, association, and re-assortment of sequence-controlled polymers in the NSO. Information retrieval is achieved rapidly by sequencing. Therefore, the methods enable rapid and efficient organization and access of sequence-controlled polymers stored within NSOs.

Methods for the storage of nucleic acids of any length, or any form have also been developed. Typically, nucleic acids of any desired length are packaged, encapsulated, enveloped, or encased in gel-based beads, protein viral packages, micelles, mineralized structures, siliconized structures, or polymer packaging, herein referred to as “nucleic acid package.” In some forms, linear nucleic acids are base-paired, double-stranded. In other forms, linear nucleic acids include a long continuous single-stranded nucleic acid polymer or many such polymers. These discrete nucleic acid packages serve as nucleic acid storage objects (NSOs) and allow incorporation of one or more specific tags on the surface of the structures. Some exemplary tags include nucleic acid sequence tags, protein tags, carbohydrate tags, and any affinity tags.

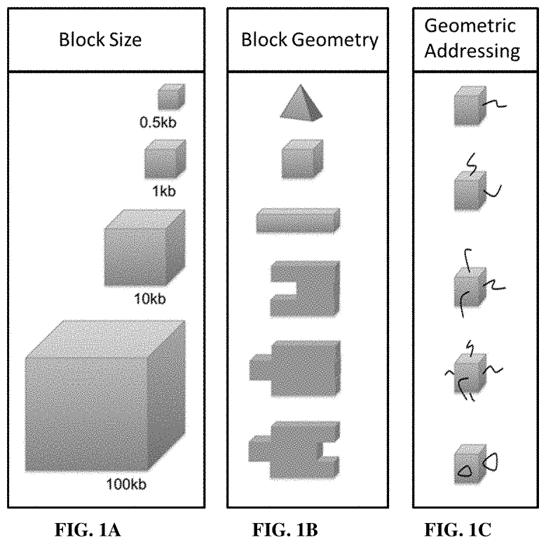

Therefore, methods for assembling sequences in the sequence of the single-strand scaffold allows for natural spatial segregation of sequence-controlled polymers, tagging or addressing the sequence-controlled polymers multiple times by functionalizing the staple strands used to fold the object, exchanging the staple strands with different overhangs to modify the address, and associating NSOs together to further spatially segregate sequence-controlled polymers of interest. Nucleic acids can be nanostructured into a diverse set of sizes and structures, and can be multiply addressed in geometrically specific positions (FIGS. 1A-1C). Nanostructured nucleic acid can fold over a wide range of scaffold sizes, from just a few hundred nucleotides up to hundreds of thousands of nucleotides in user-defined highly specific geometries that are theoretically unlimited in size. Single-stranded scaffolds can be used as a scaffold that is routed through an object that is folded to a specific shape by complementary single-strand oligonucleotide staples, or alternatively by programming the single-stranded scaffold sequence to fold onto itself. These shapes can adopt any desired arbitrary form, for example, as defined by the user. In some forms the structures are closed tightly packed blocks. In other forms the structures have the form of an open wireframe mesh, for example, a polyhedral structure. In each case, the geometry of the structures can be prescribed in an arbitrary manner to suit overall storage block super-structuring and tag presentation/accessibility.

### 2. Sequence-Controlled Polymer Storage Access

Methods of sorting, organizing and accessing sequence-controlled polymers within SSOs amongst a pool of different SSOs are described. Typically, the methods select and sort SSOs based upon inter-molecular interactions between differently or equally addressed SSOs in the pool. Typically, the methods employ nucleic acid labels bound to specifically to one or more SSOs. In some forms each SSO contains a single tag. In other forms, each SSO contains more than a single tag. Therefore, in some forms the methods provide multiply-addressed SSOs. Multiply-addressed SSOs allow rapid selection of nucleic acids using user-defined combinations of Boolean logics including AND, OR, and NOT logic. In some forms, the methods employ nucleic acid labels to physically associate distinct SSOs to one another. Therefore, in some forms the methods provide systems for rapid retrieval using the previous logic and enable physical association in supra-storage blocks for networking and spatially segregating blocks of related sequence-controlled polymers. In other forms, storage blocks are geometrically positioned in a specific location that allows for co-ordination of storage locations.

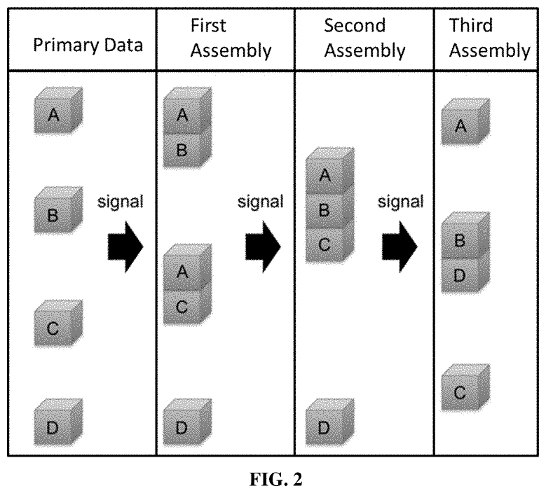

SSOS, including nanostructured NSOs, can be associated into larger super-structures based on signals to a pool of storage objects (FIGS. 2A-2D). In some forms a pool of SSOs contained in a solution is assembled based on specific geometries of overhang sequences in precise locations. Typically, assembly occurs through complementary sequences on overhangs, through a bridging oligonucleotide (splint strand), or through protein or chemical adducts to overhangs. The super-structured SSOs can be specifically dissociated and re-grouped by using external signals as desired by the user. Exemplary external signals used to control dissociation include changing the pH, lowering the salt, increasing the temperature, application of electro-magnetic radiation, toe-hold strand displacement, complementary strand excess, or enzymatic release by restriction nucleases, nickases, helicases, resolvases, releasing using UV-sensitive linker, using CRISPR/Cas9 and guide RNAs, or any combination thereof.

Sequence-controlled polymers can be biopolymers, such as DNA or polypeptides, or synthetic biopolymers, such as peptidomimetics.

A non-limiting list of suitable sequence-controlled polymers includes naturally occurring nucleic acids, non-naturally occurring nucleic acids, naturally occurring amino acids, non-naturally occurring amino acids, peptidomimetics, such as polypeptides formed from alpha peptides, beta peptides, delta peptides, gamma peptides and combinations, carbohydrates, block co-polymers, and combinations thereof. Sequence-defined unnatural polymers closely resemble biopolymers, such as polymers incorporating non-canonical amino acids. e.g., peptidomimetics, such as β-peptides (Gellman, S H. Acc. Chem. Res., 31, 173-180 (1998)), peptide nucleic acids (PNA), peptoids or poly-N-substituted glycines (Zuckermann, et al., J. Am. Chem. Soc., 1 14, 10646-10647(1992)), Oligocarbamates (Cho, C Y et al., Science, 261, 1303-1305(1993), glycomacromolecules, Nylon-type polyamides, and vinyl copolymers.

Enzymatic and non-enzymatic synthesis of sequence-defined non-natural polymers can be achieved through templated polymerization (reviewed in Brudno Y et al., Chem Biol.; 16(3): 265-276 (2009)).

In some forms, the methods include providing a nucleic acid sequence from a pool containing a multiplicity of similar or different sequences is provided. In some forms, the pool is a database of known sequences. For example, in certain forms a discrete “block” is contained within a pool of nucleic acid sequences ranging from about 100-1,000,000 bases in size, though this upper limit is theoretically unlimited. In some forms, the nucleic acid sequences within a pool of multiple nucleic acid sequences share one or more common sequences. When nucleic acids that are provided are selected from a pool of sequences, the selection process can be carried out manually, for example, by selection based on user-preference, or automatically.

B. Constructing SSOs

Generally, the goal of generating individual SSOs is to segregate blocks of sequence-controlled polymers from other blocks and to separate the identifying tags from the underlying sequence-controlled polymers and to allow large packages to be manipulated and selected as needed.

1. Custom Design of SSOs by Encapsulating Sequence-Controlled Polymers

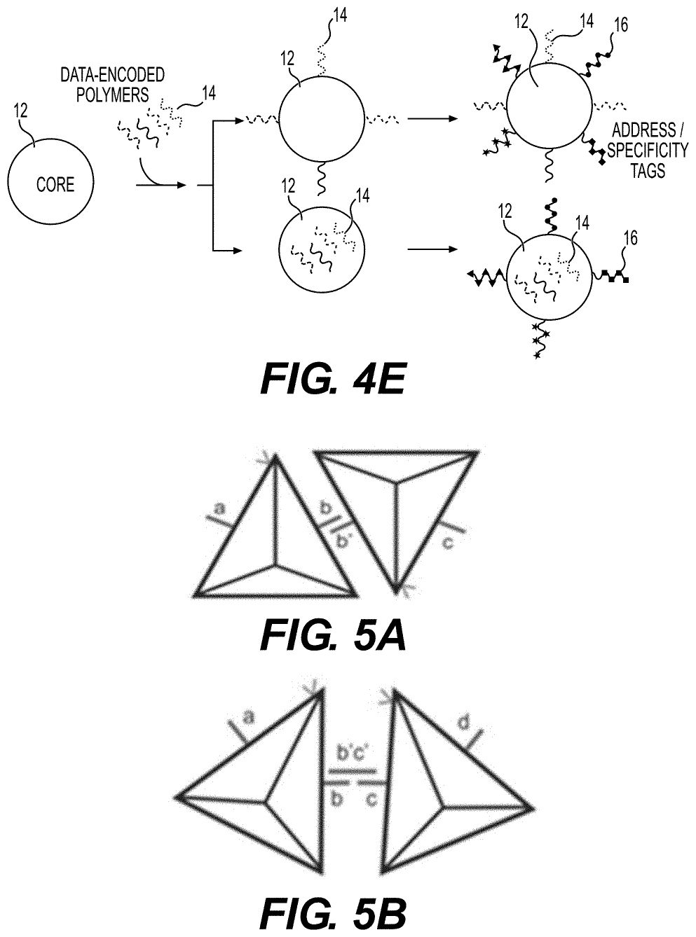

Sequence-controlled polymers can be formed into SSOs by way of encapsulation (FIGS. 4A-4E, FIGS. 19A-19C, FIGS. 20A-20B, and FIGS. 21A-21B). For example, single- and or double-stranded DNA, or any other nucleic acid can be used to generate NSOs by way of encapsulation. Sequence-controlled polymers to be encapsulated can take any arbitrary form, for example, a linear DNA sequence, a two-dimensional DNA object or a three-dimensional DNA object, a polypeptide, a protein, etc. In some forms, the linear polymers are nucleic acids that are base-paired and double stranded. In other forms, the linear nucleic acids include a long continuous single-stranded nucleic acid polymer or many such polymers. In further forms, nucleic acids encapsulated within the same particle are a mixture of linear, and non-linear nucleic acids. For example, one or more single-stranded nucleic acids and one or more scaffolded nucleic acid nanostructure can be encapsulated within the same particle.

In some forms, sequence-controlled polymers are packaged into discrete SSOs via encapsulation. Suitable encapsulating agents include gel-based beads, protein viral packages, micelles, mineralized structures, siliconized structures, or polymer packaging.

In some forms, the encapsulating agents are viral capsids or a functional part, derivative and/or analogue thereof. In some forms, the encapsulating agents are lipids forming micelles, or liposomes surrounding the nucleic acid. In some forms, the encapsulating agents are natural or synthetic polymers. In some forms, the encapsulating agents are mineralized, for example, calcium phosphate mineralization of alginate beads, or polysaccharides. In other forms, the encapsulating agents are siliconized. Packaging of sequence-controlled polymer sequences into storage blocks allows for selection and superstructuring by use of molecular identifiers, or “addresses.” In addition to nucleic acid overhangs, other purification tags can be incorporated into the overhang nucleic acid sequence in any SSOs for purification (i.e. sequence-controlled polymer retrieval). In some forms, the overhang contains one or more purification tags. In some forms, the overhang contains purification tags for affinity purification. In some forms, the overhang contains one or more sites for conjugation to a nucleic acid, or non-nucleic acid molecule. For example, the overhang tag can be conjugated to a protein, or non-protein molecule, for example, to enable affinity-binding of the SSOs. Exemplary proteins for conjugating to overhang tags include biotin, antibodies, or antigen-binding fragments of antibodies.

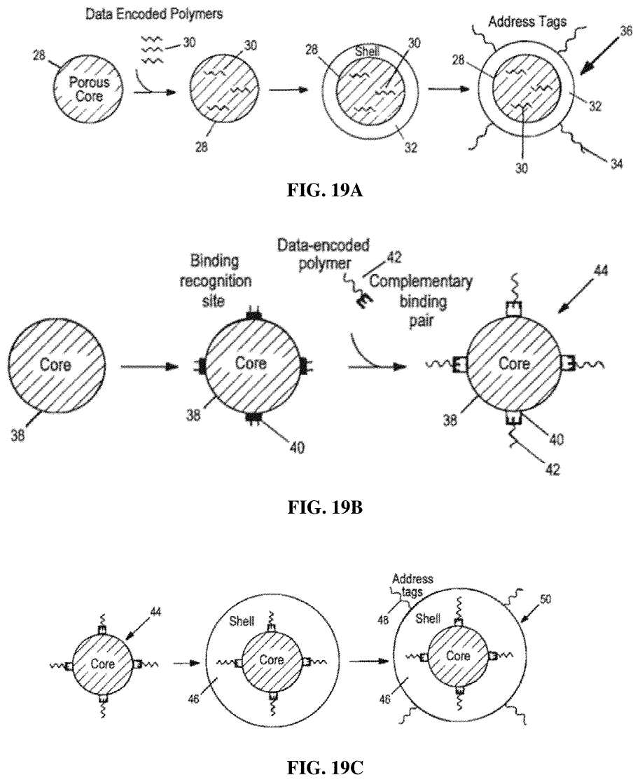

Assembly of storage objects by encapsulation, or direct assembly of sequence-controlled polymers and feature tags can be carried out to produce storage objects having a range of different structures. For example, in some forms, storage objects include a core particle, onto which one or more sequence-controlled polymers is bound. Binding of sequence-controlled polymers to a particle core can be achieved using covalent or non-covalent linkages. In some forms, a core molecule is coated or coupled to a molecule which is an intermediary receptor, for example, a binding site that is recognized by one or more ligands associated with the sequence-controlled polymer (see FIG. 19B). sequence-controlled polymers can be coupled or hybridized to the receptor-coated core molecule. In some forms, the polymer/core substructure is then coated with one or more encapsulating agents (i.e., “molecular shelling”) to produce a coated polymer/core structure, which is then coupled to one or more feature tags (see FIG. 19C). Binding of feature tags to a coated polymer/core particle can be achieved using covalent or non-covalent linkages, or hybridization of complementary nucleic acids.

In some forms, assembly of a storage object includes loading or complexing one or more sequence-controlled polymers within the interior space(s) of a porous, or otherwise accessible polymer core molecule or structure (see FIG. 19A). In some forms, assembly of a storage object includes encapsulating, or shelling the polymer-loaded core to create an encapsulated polymer-loaded particle, which is then complexed with one or more feature tags.

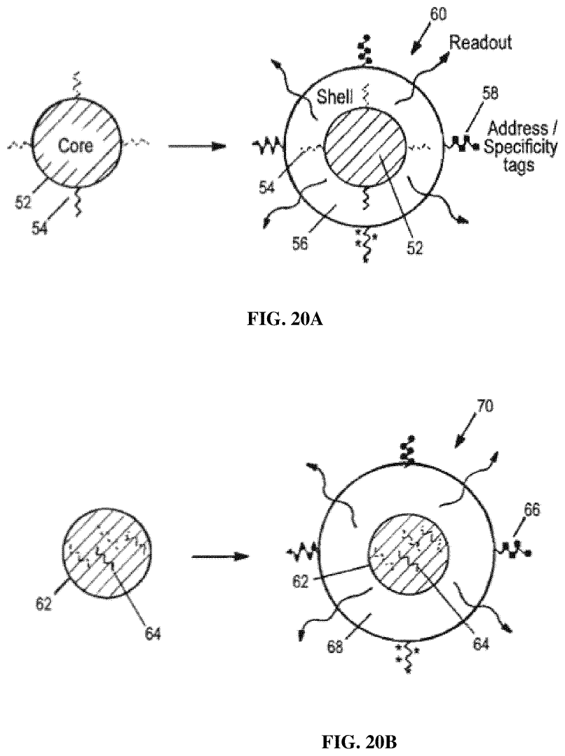

In some forms, storage objects include a sequence-controlled polymer, and optionally core molecules and/or encapsulating agents that are coated with multiple different types of feature tags. For example, in some forms, storage objects are assembled to enable multiplexed molecular logic operations and sequence-controlled polymer selection. For example, in some forms, encapsulation or molecular shelling of one or more sequence-controlled polymers, including multiple pieces of sequence-controlled polymers are labelled with multiple feature tags. The feature tags can be attached directly to the molecular core or absorbed by a molecular core are further surrounded by a molecular shell and functionalized with addressing/specificity tags for multiplexed computation (FIGS. 20A-20B).

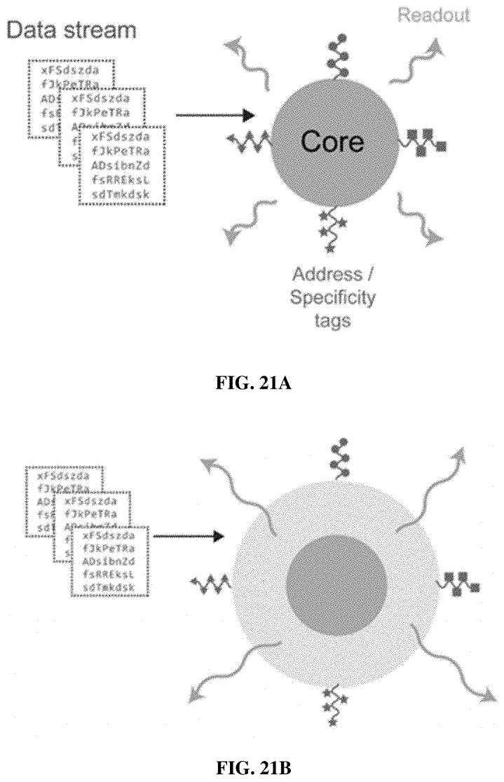

In some forms, storage objects include a sequence-controlled polymer, and optionally core molecules or encapsulating agents that are coated with feature tags, which are then coated with a shell or core which itself produces a signal or has another property that can be detected and measured to produce a readout. The outer “shell,” or inner “core” of a storage particle can, therefore, be used to address or label the storage object. Exemplary physical or chemical properties that can be detected and measured include optical, magnetic, electric, or physical properties. Therefore, in some forms, the outer shell or inner core of a storage object produces a readout based on optical, magnetic, electric, or physical properties of the shell/core. FIGS. 21A-21B are schematic illustrations depicting storage wherein sequence-controlled polymers are in the molecular core or shell. Therefore, in some forms, sequence-controlled polymers are emplaced directly on a molecular core, which has a readout based on optical, magnetic, electric, or physical properties of the core. The molecular core also contains address/specificity tags for molecular logic and sequence-controlled polymer retrieval operations. In some forms, the sequence-controlled polymers are on a molecular shell surrounding a molecular core. The shell/core has readouts based on the optical, magnetic, electric, or physical properties of the shell/core. The shell is functionalized with addressing/specificity tags for molecular logic and sequence-controlled polymer retrieval operations. In some forms, the core structure of the particle is formed from the sequence-controlled polymer folded into a 3D polyhedral or 2D polygon shape. For example, in some forms, the sequence-controlled polymer is a nucleic acid, which is folded into a nucleic acid nanostructure having a 2D or 3D shape, which is appended with one or more feature tags. Therefore, in some forms, the shape of a nucleic acid nanoparticle can be used to identify, sort or select the sequence-controlled polymers in the storage object. In some forms, the nucleic acid nanoparticle contains one or more additional core or encapsulating molecules that has a readout based on optical, magnetic, electric, or physical properties of the core.

### i. Nucleic Acid Nanostructures

Two general approaches of constructing nucleic acid storage objects (NSOs) are described below: (1) using scaffolded nucleic acid(s) along with their associated staple strands; (2) using encapsulating material to encase a defined amount of nucleic acids into a single NSO unit. Scaffolded nucleic acid nanostructures are therefore primarily made of nucleic acids, although additional non-nucleic acid component(s) can be added to the overhang sequence, for example, a protein tag for purification, or a nuclease for degradation of the nucleic acid. Encapsulated nucleic acid units can be made of any natural or synthetic materials. In some forms, scaffolded nucleic acid nanostructures are also encapsulated in one or more layers of polymers for additional layers of addresses/metadata tags, and/or for long-term stability.

### a. Scaffolded Nucleic Acid

The methods include assembling sequence-controlled polymers into a nucleic acid nanostructure. Many known methods are available to make scaffolded nucleic acid, such as DNA origami structures. Exemplary methods include those described by Benson E et al (Benson E et al., Nature 523, 441-444 (2015)), Rothemund P W et al (Rothemund P W et al., Nature. 440, 297-302 (2006)), Douglas S M et al., (Douglas S M et al., Nature 459, 414-418 (2009)), Ke Y et al (Ke Y et al., Science 338: 1177 (2012)), Zhang F et al (Zhang F et al., Nat. Nanotechnol. 10, 779-784 (2015)), Dietz H et al (Dietz H et al., Science, 325, 725-730 (2009)), Liu et al (Liu et al., Angew. Chem. Int. Ed., 50, pp. 264-267 (2011)), Zhao et al (Zhao et al., Nano Lett., 11, pp. 2997-3002 (2011)), Woo et al (Woo et al., Nat. Chem. 3, pp. 620-627 (2011)), and Torring et al (Torring et al., Chem. Soc. Rev. 40, pp. 5636-5646 (2011)), which are incorporated here in the entirety by reference.

Typically, creating a NSO includes one or more of the steps of

(1) Designing the NSO;

(2) Labelling the NSO;

(3) Assembling the NSO; and

(4) Purifying the Assembled NSO.

### b. Custom Design of Nucleic Acid Nanostructures

The nucleic acid nanostructure has a defined shape and size. Typically, one or more dimensions of the nanostructure are determined by the target sequence. The methods include designing nanostructures including the target nucleic acid sequence.

Nucleic acid nanostructures for use as NSOs can be geometrically simple, or geometrically complex, such as polyhedral three-dimensional structures of arbitrary geometry. Any methods for the manipulation, assortment or shaping of nucleic acids can be used to produce NSO nanostructures. Typically, the methods include methods for “shaping” or otherwise changing the conformation of nucleic acid, such as methods for DNA origami.

In some forms, nanostructures of nucleic acid target sequences are designed using methods that determine the single-stranded oligonucleotide staple sequences that can be combined with the target sequence to form a complete three-dimensional nucleic acid nanostructure of a desired form and size. Therefore, in some forms, the methods include the automated custom design of nucleic acid storage objects (NSOs) corresponding to a target nucleic acid sequence. For example, in some forms, a robust computational approach is used to generate DNA-based wireframe polyhedral structures of arbitrary scaffold sequence, symmetry and size. In particular forms, design of a NSO corresponding to the target nucleic acid sequence, includes providing geometric parameters corresponding to the desired form and dimensions of the NSO, which are used to generate the sequences of oligonucleotide “staples” that can hybridize to the target nucleic acid “scaffold” sequence to form the desired shape. Typically, the target nucleic acid is routed throughout the Eulerian circuit of the network defined by the wire-frame geometry of the nanostructure of the nanostructure.

Therefore, in some forms, a NSO is designed by a method including the steps of:

(1) Selecting a target structure, which may be from a predefined set of geometries, or may additionally include the steps of:
(a) Determining the spatial coordinates of all vertices, the edge connectivities between vertices, and the faces to which vertices belong in the target structure;
(b) Identifying the route of a single-stranded nucleic acid scaffold sequence that traces throughout the entire target structure, and

(2) Determining the nucleic acid sequence of the single-stranded nucleic acid scaffold and the nucleic acid sequence of corresponding staple strands.

A step-wise, top-down approach has been proven for generating DNA nanostructure origami objects of any regular or irregular wireframe polyhedron, with edges composed of a multiple of two number of helices (i.e., 2, 4, 6, etc.) and with edge lengths a multiple of 10.5 rounded down to the closest integer.

Typically, the route of the scaffold nucleic acid is identified by

(i) Determining edges that form the spanning tree of the node-edge network (for example, using the Prim's Algorithm);

(ii) Bisecting each edge that does not form the spanning tree to form two split edges;

(iii) Determining an Eulerian circuit that passes twice along each edge of the spanning tree. The direction of the continuous scaffold sequence is reversed at the bisecting point of the node-edge network in a DX-anti-parallel crossover, and the Eulerian circuit defines the route of a single-stranded nucleic acid scaffold sequence that passes throughout the entire structure. In some forms, the spanning tree that is used to determine positions of the scaffold crossovers for the scaffold routing is a maximum breadth spanning tree. This is important in minimizing the number of staples per object, leading to a more stable/robust structure. Any spanning tree, however, will lead to a valid scaffold routing. In some forms, this method is implemented as a computational tool.

Given inputs of the geometry of the nanoparticle and the scaffold sequence the program output is of the staple sequences necessary to fold the scaffold into the chosen nanoparticle. Staple strands are located at the vertices and edges of the route of the single-stranded nucleic acid scaffold sequence determined in (3). In some forms, these staple oligonucleotide sequences have nick positions where either a staple strand closes in on itself or where two staple strands come together, and the nick strands are positioned to be away from the center of the object (“outside”).

Exemplary methods for the top-down design of nucleic acid nanostructures of arbitrary geometry are described in Venziano et al, Science, 352 (6293), 2016, the contents of which are incorporated by reference in entirety.

In other forms, the sequence of the NSO is designed manually, or using alternative computational sequence design procedures. Exemplary design strategies that can be incorporated into the methods for making and using NSOs include single-stranded tile-based DNA origami (Ke Y, et al., Science 2012); brick-like DNA origami, for example, including a single-stranded scaffold with helper strands (Rothemund, et al., and Douglas, et al.); and purely single-stranded DNA that folds onto itself in PX-origami, for example, using paranemic crossovers.

Alternative structured NSOs include bricks, bricks with holes or cavities, assembled using DNA duplexes packed on square or honeycomb lattices (Douglas et al., Nature 459, 414-418 (2009); Ke Y et al., Science 338: 1177 (2012)). Paranemic-crossover (PX)-origami in which the nanostructure is formed by folding a single long scaffold strand onto itself can alternatively be used, provided bait sequences are still included in a site-specific manner. Further diversity can be introduced such as using different edge types, including 6-, 8-, 10, or 12-helix bundle. Further topology such as ring structure is also useable for example a 6-helix bundle ring.

### c. Assembling Nucleic Acid Nanostructures

The methods include assembly of the single-stranded nucleic acid scaffold and the corresponding staple sequences into a NSO nanostructure having the desired shape and size. In some forms, assembly is carried out by hybridization of the staples to the scaffold sequence. In other forms, NSOs include only of single-stranded DNA oligos. In further forms the NSOs include a single-stranded DNA molecule folded onto itself. Therefore, in some forms, the NSOs are assembled by DNA origami annealing reactions.

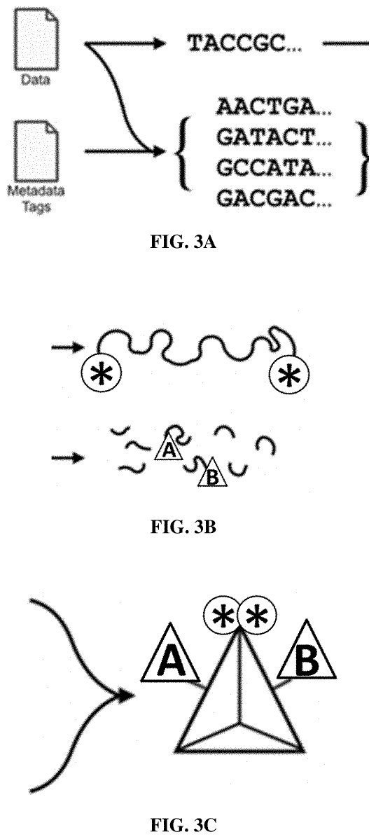

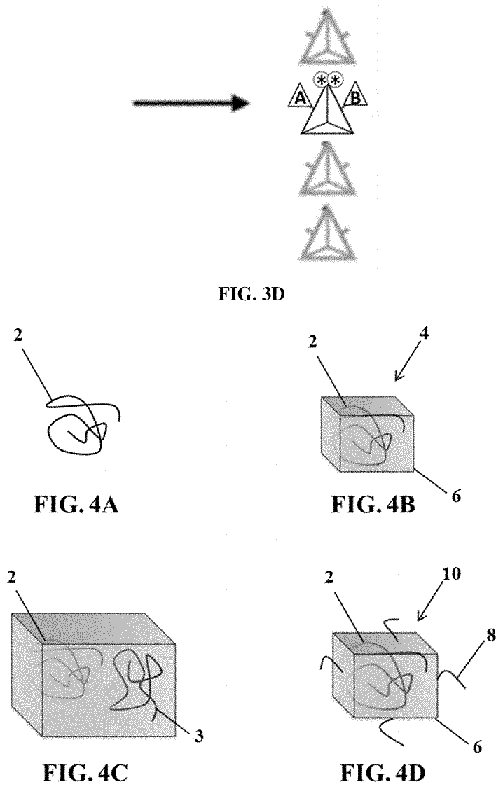

Typically, annealing can be carried out according to the specific parameters of the staple and/or scaffold sequences. For example, the oligonucleotide staples are mixed in the appropriate quantities in an appropriate reaction volume. In preferred forms, the staple strand mixes are added in an amount effective to maximize the yield and correct assembly of the nanostructure. For example, in some forms, the staple strand mixes are added in molar excess of the scaffold strand. In an exemplary form, the staple strand mixes are added at a 10-20× molar excess of the scaffold strand. In some forms, the synthesized oligonucleotides staples with and without tag overhangs are mixed with the scaffold strand and annealed by slowly lowering the temperature (annealing) over the course of 1 to 48 hours. This process allows the staple strands to guide the folding of the scaffold into the final NSO. This is done either in separate wells and added to a pool of NSOs (as in FIGS. 3A-3D), or in a pool of oligonucleotides and scaffolds to generate a pool of NSOs. In FIGS. 3A-3D, an exemplary NSO is shown as a tetrahedron, representative of any storage block.

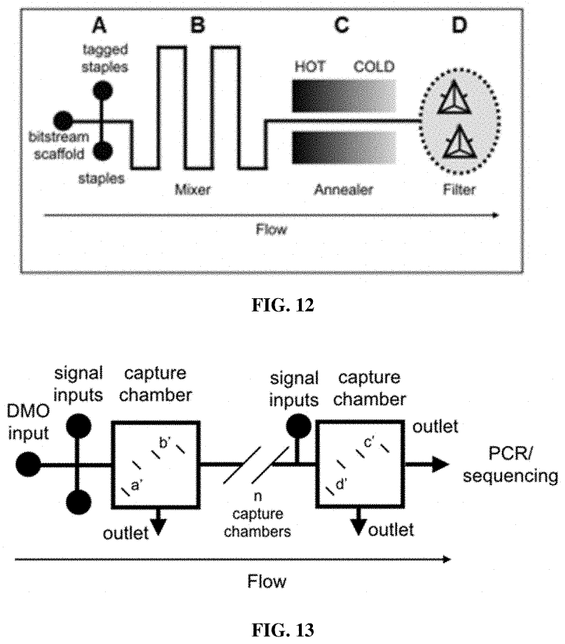

Material usage for assembly can be minimized and assembly hastened by use of microfluidic automated assembly devices (FIGS. 11-12). For example, in certain forms, the oligonucleotide staples are added in one inlet, the scaffold can be added in a second inlet, with the solution being mixed using methods known in the art, and the mix traveling through an annealing chamber, wherein the temperature steadily decreases over time or distance. The output port then contains the assembled NSO for further purification or storage. Similar strategies can be used based on digital droplet-based microfluidics on surfaces to mix and anneal solutions and applied to purely single-stranded oligo-based NSOs or single-stranded scaffold origami in the absence of helper strands.

2. Labelling SSOs

One or more specific labels, such as nucleic acid sequence motifs, unique sequence identifiers, or “tags,” are associated with the sequence-controlled polymers on a SSO. For example, in some forms, one or more labels are selected and then encoded into a nucleic acid sequence using a conversion method of the user's choice.

Typically, the label is a nucleic acid sequence motif, such as a barcode sequence. In some forms the label includes a mechanism of direct conversion, including, but not limited to, strings, integers, dates, times, events, genres, metadata, participants, hashes, or authors. In certain forms, tags enable direct sequence selection, with the user keeping an external library of addresses.

Nanostructuring the sequence-controlled polymer blocks allows for a natural extension to spatial segregation of sequence-controlled polymers based on input signals, associating related sequence-controlled polymers into supra-block storage. The address space is multiplied by the number of tags in use. For example, the methods enable nucleotide addresses having 4^{(k*n)} bases, where n is the number of nucleotides of the address per tag and k is the number of tags. The number of tags per nanostructure can be determined by the user. Typically, each nanostructure has at least one tag, for example 2 or more tags, 3 or more tags, up to 10 tags, 20 tags, 100 tags or 1,000 tags. In some forms, each edge of a polyhedron has one tag, or more than one tag. In some forms SSOs have a number of tags that is directly proportional to the size of the polyhedron, or is dependent upon the shape of the polyhedron.

In some forms, when nanostructured nucleic acid objects are used as NSOs, the label is a nucleic acid sequence that is associated with a staple sequence in the form of an overhang “tag” sequence. Exemplary overhang sequences are between 4 and 60 nucleotides. In some forms, these overhang tag sequences are placed on the 5′ end of any of the staples used to generate a wireframe DNA. In other forms, these overhang tag sequences are placed on the 3′ end of any of the staples used to generate a wireframe DNA. In some forms, combinations of overhangs are employed to make logic AND/OR gates to self-assemble SSOs.

In certain forms parameters including the size, charge, conformation and sequence of an overhang tag is determined by one or more of user preference, location on the SSO, downstream purification techniques, or combinations. Typically, overhang tag sequences contain metadata for the scaffolded nucleic acid. For example, overhang tag sequences have address(es) for locating a particular sequence-controlled polymer. In some forms, each overhang tag contains a plurality of functional elements such as addresses, as well as region(s) for hybridizing to other overhang tag sequences, or to bridging strands.

In some forms, the total maximal number of tags per individual NSO from 1 overhang is up to 2× (number of staples in the NSO). For example, one staple has one tag, or two tags; two staples have one tag, two tags, three tags, or four tags and so on. These tag sequences added to the staple sequences at user-defined locations, with the untagged staple strands are then synthesized individually or as a pool directly using any known methods.

In some forms, the tag is designed to change one or more of the interactions between the tag and the scaffold nucleic acid with which it interacts. In some forms the nucleic acid sequence of the tag is designed or manipulated by appending one or more sequences that alter the physical properties of the tag. Exemplary physical properties of the nucleic acid sequence that can be modified include the melting temperature or the nucleic acid. For example, in some forms, the melting temperature and length of the nucleic acid sequence is controlled such that hA the total length, or more than A of the total length of the sequence is the hash value and the other half of the sequence is a “homo-typic” sequence including one type of nucleotide, or a randomly or non-randomly generated permutation of two types of nucleotides, or three types of nucleotide, or greater than three types of nucleotides. In an exemplary form, the melting temperature and length of a DNA sequence is controlled such that hA the length of the sequence is the hash value and the other half of the sequence is composed of nucleotides that make the GC content 50% and an 18-mer in length.

Other physical features of the tag that can be varied include the secondary structure of the nucleic acid, the ratio of one or more types of nucleotides relative to one or more of the other types of nucleotides, or the length, molecular weight, or electrochemical properties of the nucleic acid sequence.

In other forms, the tag sequence is a category with discrete values. Exemplary discrete values include any integer value, such as year, or collection of integer values, such as date. In other forms, the tag sequence encodes some continuous variable such as a shade of blue. In some forms the tag is partially used for key storage and partially used for value storage such that a value-key pair is stored on the tag.

In some forms, the pools contain different sets of tag overhangs for the same object, such that a single sequence-controlled polymer is addressed with many times the number of allowed functional nick positions in the object itself. In some forms, the scaffold polymer is overlapped in sequence with multiple other scaffold messages to allow for bioinformatics assembly of long messages that extend beyond the size of the scaffold of the chosen geometries.

3. Purifying Assembled SSOs

The methods include purification of the assembled SSOs. Purification separates assembled structures from the substrates and buffers required during the assembly process. Typically, purification is carried out according to the physical characteristics of nanostructures, for example, the use of filters and/or chromatographic processes (FPLC, etc.) is carried out according to the size and shape of the nanostructures.

In an exemplary form, SSOs are purified using filtration, such as by centrifugal filtration, or gravity filtration, or by diffusion such as through dialysis. In some forms, filtration is carried out using an Amicon Ultra-0.5 mL centrifugal filter (MWCO 100 kDa).

C. Storing Information as SSOs

The methods include storage of SSO structures. Purified SSOs can be placed into an appropriate buffer for storage, and/or subsequent structural analysis and validation.

In some forms the SSOs are stored in solution. In an exemplary form, SSOs are stored in an aqueous solution. Suitable aqueous storage buffers include PBS, and TAE-Mg²⁺. In other forms, SSOs are stored in oil, or an emulsion, or other hydrophobic solution. In some forms, the SSOs are dried or dehydrated, for example by lyophilization. In certain forms, the SSOs are dried and affixed to a solid support, such as filter paper.

Storage can be carried out at room temperature (i.e., 25° C.), 4° C., or below 4° C., for example, at −20° C., −40° C. or −80° C. In some forms, the NSOs are frozen, for example by immersion in liquid nitrogen.

In some forms, SSOs are stored at conditions for desired longevity. For example, the nucleic acid within NSOs can be maintained at high-fidelity for prolonged periods of time. For example, in some forms, NSOs are stored for up to a day, more than a day, up to a week, more than a week, up to a month, up to six months, up to a year, more than a year, up to 2 years, 3 years, 5 years, 10 years, more than 10 years, up to 20 years, or more than 20 years. Typically, very little energy required for maintenance (Zhirnov, V et al., Nature materials. 15, 366-370 (2016)). Typically, NSOs maintain the fidelity of information encoded within the nanostructures or encapsulated for a period of time that is greater than tape-based storage having a life-time rating of 10-30 years.

DNA's information retention has been improved to an estimated ˜2,000 years at 10° C. and ˜2,000,000 years at −18° C. by the encapsulation of the DNA in silica (Grass, R N et al., Angew. Chem. Int. Ed. 54, 2552-2555 (2015)).

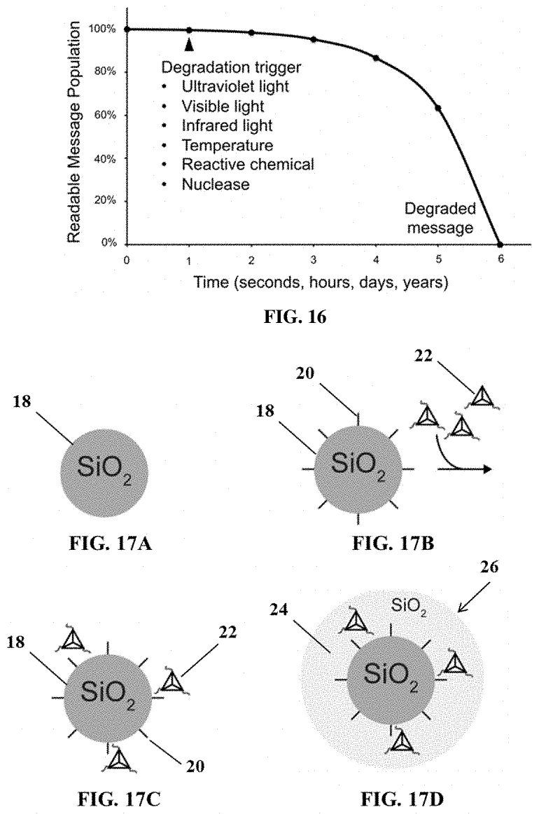

In some forms, the SSOs are preserved by chemical means, for example, encapsulation in silica (SiO₂). For example, in some forms, NSOs are preserved by chemical means, for example, encapsulation in silica (SiO₂). Therefore, redundancy of sequence-controlled polymer storage can be used to ensure that replicates of NSOs that may degrade over time in a random manner where nucleotide identity is lost can still be read out to reconstruct overall storage. Sequencing errors can also be eliminated by reading multiple copies of NSOs and using consensus sequence mapping. Degradation of nucleic acid storage objects upon exposure to external stimuli is depicted in FIG. 16.

D. Sequence-Controlled Polymers as SSOs

The methods enable the organization of sequence-controlled polymers contained within SSOs. Typically, organization of sequence-controlled polymers is carried out by separating, associating or otherwise partitioning one sequence-controlled polymer with or from another sequence-controlled polymer. Therefore, in some forms, the methods organize sequence-controlled polymers by association or separation of one or more SSOs.

In some forms organization of sequence-controlled polymers is achieved by physical manipulation of one or more SSOs within a pool of SSOs.

1. Association of SSO Superstructures

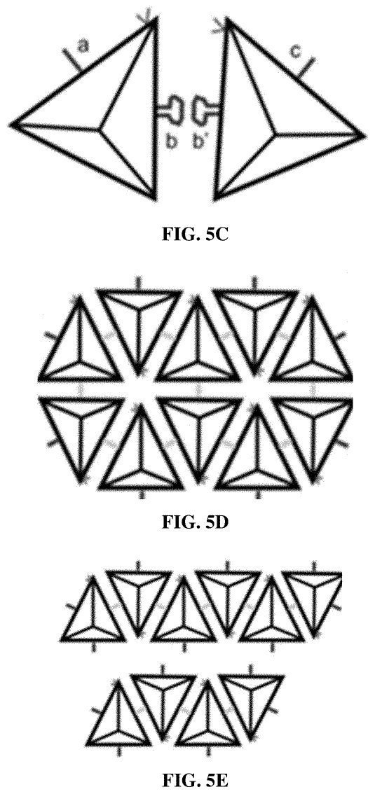

In some forms, the methods group or otherwise connect sequence-controlled polymers by physically associating two or more SSOs to form SSO superstructures. Therefore, the methods allow association of larger sets of SSOs. An exemplary super-structure is shown in FIGS. 5D-5E, where 10 tetrahedra are associated together. In an exemplary form, two tetrahedral storage objects are associated and four tetrahedral storage objects are brought together in a dimer and tetramer of SSOs in a complex, respectively, by way of two complementary overhangs per edge. Such association techniques are not limited to tetrahedra i.e. any nucleic acid storage object with a larger or smaller set of objects in the super-structure. Association through staple tags typically involves complementary tag sequences, bridging or splint sequences, kissing loops, or hybrid interconnecting staple strands, or hybrid interconnecting staple strands. In some forms, association occurs based on structural complementarity and non-specific base-stacking of DNA duplex ends, to form larger-scale 1D/2D/3D semi-crystalline or crystalline arrays in solution or on surfaces. Typically, buffer conditions and temperature are used to control the aggregation state of such non-specifically associated SSOs.

### i. Complementary Tag Sequences

In some forms, SSO structures chosen for association by the user are assembled such that their tag overhangs of two objects to be associated are complementary in their nucleotide sequences. As the objects with the complementary sequences are brought together, the overhang sequences anneal and the objects will form larger superstructures. An exemplary complementary tag interaction between two NSOs is depicted in FIG. 5A.

### ii. Bridging or Splint Sequences

In some forms, two objects are brought together with two non-complementary tag overhang sequences using a bridging or splint oligonucleotide, which contains complementary nucleotide sequence to the two overhang sequences. This allows for more dynamic associations, as the splint strand is added later after the folding of the individual objects. An exemplary bridging interaction between two NSOs is depicted in FIG. 5B.

### iii. Interconnecting Staples

In further forms, two SSO structures are assembled using a hybrid staple that directly acts as a staple between two storage scaffolds, bringing the objects together directly during folding. In this case, the SSOs are stably bound to each other.

### iv. Kissing Loops

In certain forms, two SSO structures are assembled using a kissing loop mechanism where complementary loops are present in two different storage objects and that directly connect two storage scaffolds, when the scaffolds are mixed together. This method brings the two objects together directly after folding. In this case, the SSOs are stably bound to each other. An exemplary kissing-loop interaction between two NSOs is depicted in FIG. 5C.

2. Dissociation of SSO Superstructures

The methods include dissociating SSO superstructures. Methods for dissociation of superstructure objects include multiple techniques, including but not limited to changing the pH, for example by increasing or decreasing pH, changing the salt concentration, increasing the temperature, toe-hold strand displacement, enzymatic release by restriction nucleases, nickases, helicases, resolvases, UV/light sensitive linkers, or any combinations thereof.

This has application in association of nucleic acid storage block structures, for example, in making a superstructure of all objects associated with the species H. sapiens, by inserting sequences that would aggregate all objects tagged with the metadata addressing the species H. sapiens. Dendritic DNA stars including arrays of single-stranded overhangs physically associated at a central covalent linkage or on a bead may also be used to aggregate SSOs in this manner.

Additionally, re-assortment of super-molecular storage structures is also feasible using nanostructured data. SSOs, which have been associated via splint strands, complementary tag overhangs, or kissing loop interactions can be dissociated via a variety of techniques, including by changing the pH, lowering the salt, increasing the temperature, toe-hold strand displacement, enzymatic release by restriction nucleases, nickases, helicases, resolvases, or any combination thereof. Re-association of the SSOs then allows for a modification in the structures of the controlled aggregates.

In the context of associative storage, this allows the re-association of new combinations of scaffolds. For example, this allows for disassembling the superstructure representing SSOs displaying metadata tags encoding the species H. sapiens and re-associating a new SSO superstructure associating all NSOs displaying metadata tags encoding for human neural DNA.

Tags from functionalized staple strands can be modified with a new addressing system, and the nanostructures can be refolded with the new set of tagged staples. This allows for a dynamic addressing system that does not require resynthesis of all the sequence-controlled polymers. Dissociation can also be used to move SSOs from one to another storage block based on extrinsic signals or cues described above. A schematic chart depicting the associative nanostructured data framework amongst a pool of nucleic acid storage objects is depicted in FIG. 2.

E. Access of Sequence-Controlled Polymers within SSOs

The methods include the step of accessing sequence-controlled polymers. For example, nucleic acid sequences can be accessed by selecting one or more SSOs, for example, selecting a subset of SSOs or SSO superstructures. Typically, selection of SSOs is carried out using methods that selectively capture or remove one or more sequence tags associated with one or more SSOs or subsets of SSOs. Therefore, the methods provide random access of information. In some forms, selection is based on SSO geometry, SSO size, SSO sequence, or combinations. In some forms, nucleic acids and/or nucleic acid structures are bound to a solid phase for use in the selection and purification of SSOs. For example, nucleic acids can be hybridized onto beads, such as AMPure XL SPRI beads.

In some forms, methods for retrieval of encapsulated sequence storage objects target one or more populations of interest for retrieval from a pool of populations. For example, in some forms, the methods retrieve encapsulated sequence storage objects including one or more populations of interest from a pool of populations, wherein the sequence storage objects include molecular tags corresponding to one or more characteristics associated with the population of interest, and wherein the retrieval includes

(i) contacting the molecular tags with molecular probes that selectively bind to the molecular tags associated with the population of interest; and

(ii) isolating the sequence storage objects bound to the probes.

To permit retrieval of collections of particles belonging to one of a number of discrete categories, one orthogonal barcode sequence is associated with each category and a particle's membership in each category is indicated by the particle's corresponding selection of barcodes. The various schemes by which barcodes may be assigned to particles in order to permit selection of different collections of related particles are also described.

1. Selection of Geometry

In some forms, when nanostructured nucleic acid objects are used as NSOs, the methods include selecting the geometry of nanostructured NSOs. Therefore, in some forms, NSOs having certain geometry are selected from a pool of NSOs having different geometry (FIGS. 7A-7C). For example, in some forms, geometry determines the position and/or accessibility of one or more tags. In some forms, NSOs having defined tags in certain orientations on the NSO allow for the specific capture of only those NSOs. In certain forms, one or more NSOs or NSO superstructures with specific sequences and geometries satisfying the specific geometric placement of complementary strands on a complementary or receiving object are selected.

For example, as shown in FIGS. 7A-7C, a nanostructured NSO displaying sequences a and b on different geometric locations, such as on two edges. These sequences would be complementary to two overhangs on a complementary geometric DNA nanostructure, displaying a′ and b′ at positions ideal for selecting the NSO. Typically, the larger nanostructure is part of a surface, or bound to a surface or solid support by chemical, hybridization, or protein interaction. In this way, a NSO is specifically selected based not just on sequence of the tagged overhang, but also on the geometry of the NSO.

2. Selection Based on Sequence

The methods include selecting one or more components of the sequence of SSOs. A mechanism to selectively retrieve only desired portions of a pool (i.e., random access) is implemented by selecting the desired sequence tag of the SSOs of interest. Methods of capturing desired DNA sequence tag are known in the art.

In some forms, the desired sequence tags are captured via nucleic acid hybridization, in which “bait” sequences are used to select the tag regions of the SSOs. In some forms, the “bait” sequences are nucleotide sequences complementary to the desired sequence tag. In some forms, the “bait” sequences are DNA molecules. In other forms, the “bait” sequences are RNA molecules. In some forms, hybridization capture is an in-solution approach. In preferred forms, hybridization capture is a solid-phase (immobilized) approach.

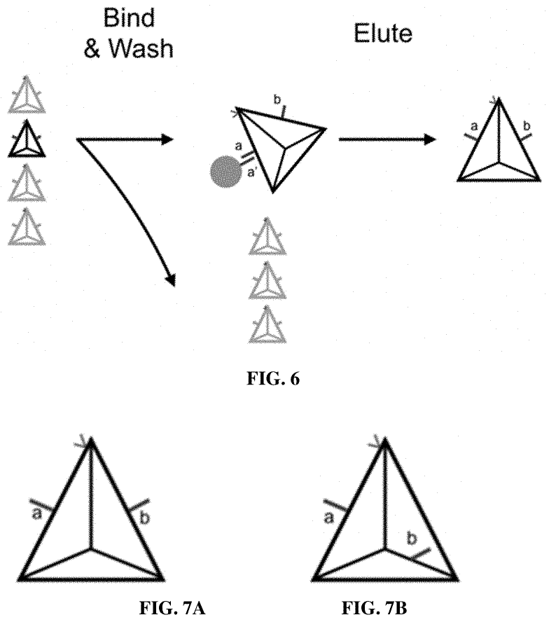

An exemplary method of retrieving NSO structures of interest from a pool of NSOs in shown in FIGS. 6A-6C. For example, in some forms a target SSO in a pool of SSOs, can be retrieved using tag overhang sequences. In some forms, short single-strand oligonucleotides are synthesized with sequences complementary to the sequence of the tag overhang of the SSOs of interest using known methods. Typically, these sequences are synthesized with a label that is used for capturing these oligonucleotides on a stationary phase, for example a biotin 5′ label. The labeled nucleotides are attached to a stationary support. Exemplary stationary supports include streptavidin-coated beads or streptavidin-coated surfaces. When biotin is used, biotin-oligonucleotide captured nucleic acids are incubated with the streptavidin support to allow for binding (hereafter “capture support”). Unbound sequences are removed from the sample, for example, by washing.

In an exemplary form, specific capture is achieved by annealing the SSO complementary overhang sequence to the capture support. Methods for specific capture of SSOs by annealing include mixing a pool of SSOs with a capture support and annealing, for example, by incubating at temperatures from 4° C. up to the melting temperature of the SSOs (approximately 55° C.), and then cooling to allow annealing. Washing the unbound fraction from the capture support using mild conditions to remove nonspecific binding, such as with slight heating or lowered salt allows for specific capture and subsequent purification of the SSO of interest away from the pool.

In some forms, the capture sequence is complementary to the key-value pair such that a target address and corresponding storage block will be captured and those target addresses with low Hamming distances and corresponding storage blocks will also be captured. Methods of increasing or decreasing this background of storage blocks with similar feature tags can be, for example but not limited to, based on temperature, pH, capture time, changes in salt. For example, an NSO with a “sky-blue” tag could be captured by a selection on a “light-blue” complementary capture support given the specific conditions of the capture.

The captured SSO is released from the capture support by any mechanisms known in the art. The non-limiting methods include changing the pH, lowering the salt, increasing the temperature, toe-hold strand displacement, enzymatic release by restriction nucleases, nickases, helicases, resolvases, or any combination thereof.

In further forms, splint strands can be generated that would include part of the sequence complementary to the tag overhang being targeted, and a second part of the splint sequence complementary to the capture sequence on the capture support, as described for superstructures in FIGS. 5A-5C.

In some forms, capturing of SSOs takes place in minimized volumes, for example, using microfluidic devices in bulk or on surfaces. In some forms a microfluidic device includes of a surface or bead-based oligonucleotide support, with sequences complementary to the tag overhang sequences of one or more SSOs. The inlet port provides an aliquot of the pooled storage objects, leading to a stationary phase capture region, allowing for segregation of capture and flow-through objects. In this manner, flow-through (i.e., unbound) objects are captured separately from the captured objects (FIGS. 13A-13G). Prior to manipulation and capture SSOs are stored in a dry state in paper, or other solid support matrix, for long-term storage prior to rehydration and manipulation prior to sequencing-based readout.

### a. Fluorescence Gate Selection

Exemplary molecular probes for use in methods for selecting and/or retrieving sequence storage objects include fluorescently labelled probes that bind selectively to molecular tags associated with the sequence storage objects. Therefore, in some forms, the methods include fluorescence gate selection. For example, in some forms, methods for isolating the sequence storage objects bound to the probes include fluorescence gate selection using different colors associated with each probe, to identify and retrieve the populations of interest.

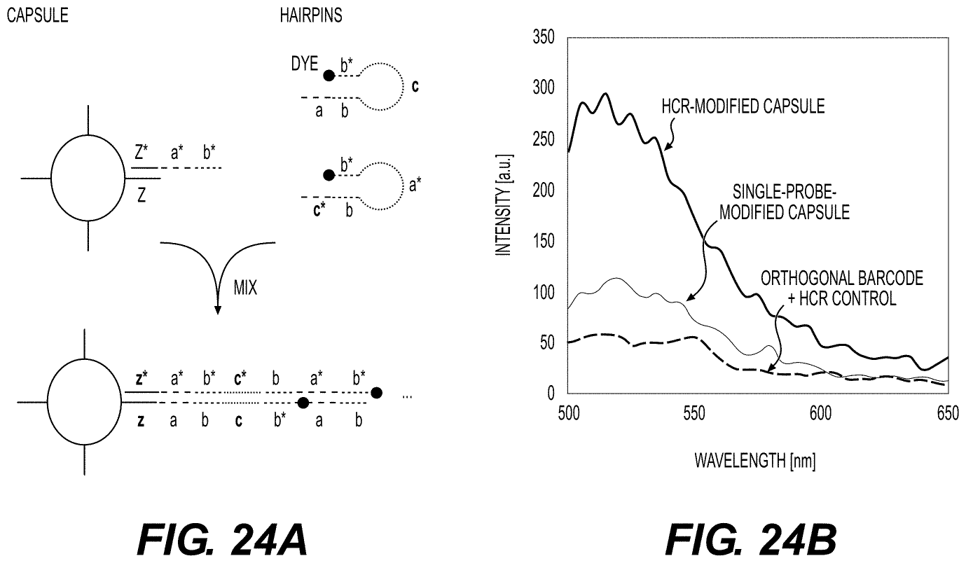

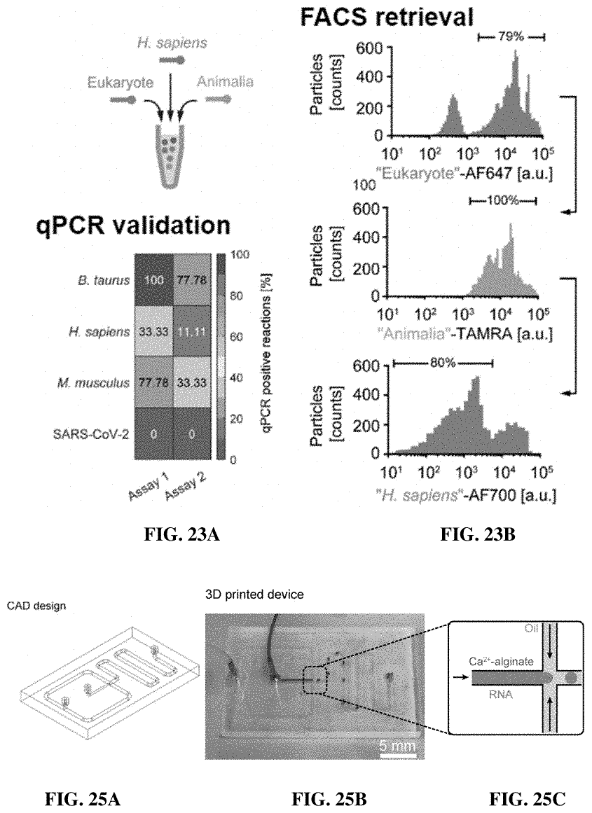

In an exemplary method for retrieval of encapsulated sequence storage objects, capsules that contain B. taurus (contains “Eukaryote”, “Animalia”, “2021-01-05”, and “Bos taurus” labels) and M. musculus (contains “Eukaryote”, “Animalia”, “2021-01-03”, and “Mus musculus” labels) genomes were targeted for retrieval from the pool that contains H. sapiens total RNA (contains “Eukaryote”, “Animalia”, “2021-01-03”, and “Homo sapiens” labels) and SARS-CoV-2 RNA genome (contains “Riboviria”, “Orthornavirae”, “2020-12-20”, and “SARS-CoV-2” labels) (see FIG. 23A). Boolean logical query using molecular probes that matches the query strings “Eukaryote”, “Animalia”, and “Homo sapiens” were added into the pool. Fluorescence gate selection using different colors associated with each probe identify the populations of interest. Selection of populations that are positive on “Eukaryote” AND “Animalia” selects B. taurus, M. musculus, and H. sapiens. Additional “Homo sapiens” gate can be used to select population that are negative for “Homo sapiens” or in Boolean logic representation, NOT Homo sapiens. Thus, the final Boolean logical search query is “Eukaryote” AND “Animalia” AND (NOT “Homo sapiens”), which selects B. taurus and M. musculus as validated using quantitative real-time polymerase chain reaction (see FIG. 23B).

### b. Hybridization Chain Reaction

In some forms, the methods also include hybridization chain reaction (HCR). For example, in some forms, methods for isolating the sequence storage objects bound to the probes include hybridization-based selection for probes designed to have distinct hybridization properties with distinct molecular “barcode” tags at the surface of sequence storage objects, to identify and retrieve the populations of interest.

In some forms, the members of at least one of the sets of feature tags are hybridization ordered, wherein the members of the at least one of the sets of feature tags have the same number of nucleotides. In some forms, in at least one of the sets of feature tags, (a) the members of the set of feature tags have the same number of nucleotides and (b) each of the feature tags in the set differs from the other feature tags in the set by 1 to x mismatched nucleotides, wherein the mismatched nucleotides are (i) at least two nucleotides from either end of the feature tag and (ii) are separated by at least one matching nucleotide in the feature tags, and wherein x is the number of different nucleotide positions in the feature tags that are varied in the set. In some forms, independently for one or more sets of the feature tags, each feature tag in the set is mismatched from every other feature tag in the set by 1 to w nucleotides, wherein w is an integer from 2 to (y−4)÷2, wherein y is the number of nucleotides in the feature tags in the set, wherein the expression (y−4)÷2 is rounded up. In some forms, the sequence-controlled storage object further includes a plurality of different digit tags, wherein the digit tags are present at the surface of the storage object, wherein the digit tags are number encoded.

Therefore, in some forms, the methods retrieve sequence storage objects including sequences of interest by hybridization-based selection of barcodes on sample surfaces as initiators. In an exemplary method, capsules that contain the “Homo sapiens” tag (e.g., labelled as “z” in FIG. 24A) are hybridized with complementary z* tag, which also includes a toehold sequence “a*” and stem sequence “b*”, triggering hybridization chain reaction (HCR) between two hairpin structure modified with a marker, which can be a dye or a chemical/biochemical tag, as depicted in FIG. 24A. When the marker is a fluorescent tag, fluorescence enhancement is observed for HCR-amplified capsules, as compared to capsules that were hybridized with only a complementary strand containing a single dye, as depicted in FIG. 24B.

### c. Numerical Range-Based Selection

In some forms, the methods include selection and/or isolation of the sequence storage objects based on or including molecular tags that are “barcodes”, where the barcode sequence design process includes a range of some numerical feature of the underlying biomolecule/sequence.

In some forms, the differences in the numerical values with which members of the set of related features have or can be associated are proportional to the similarity of the features in the set of related features. In some forms, the multidigit number is arbitrarily assigned to the feature attributable to one or more of the different sequence-controlled polymers to which the multidigit number corresponds. In some forms, the multidigit number is the same as a given number of digits of the numerical value of the feature attributable to one or more of the different sequence-controlled polymers starting from the most significant digit of the numerical value.

In some forms, each set of digit tags has the same number of members as the mathematical base in which the multidigit number is expressed. To permit retrieval of collections of particles belonging to ranges of a discretized numerical feature, one orthogonal barcode sequence is associated with each possible digit value at each digit of the number. With this approach, a collection of particles corresponding to any numerical range of the feature may be retrieved, as long as this range can be specified by selecting for particular digit values at some subset of the digits in the number. For example, in some forms, each possible digit value at each digit place of the number is associated with a distinct orthogonal barcode, permitting retrieval of ranges of values by selecting particles with particular digit values at a subset of the digit places.

As an example, a numerical feature can be represented in base 3, and the collection of particles with barcodes corresponding to numbers in the range [1000, 1100) can be retrieved by selecting particles with the barcode associated with “1” in the 27 s place and “0” in the 9 s place, as depicted in FIG. 26.

### d. Design of Sequence Tags for Exact and Approximate Similarity-Based Retrieval

In some forms, the methods also include selection and/or isolation of the sequence storage objects based on or including molecular tags that are “barcodes”, where the barcode sequence design process enables exact similarity-based retrieval with respect to a feature whose similarity metric is simple enough to permit an exact isometric embedding from feature similarity space to a low-dimensional hypercube. For example, in some forms, selection of and/or isolation of the sequence storage objects is based on similarity, determined by isometric embedding to a low-dimensional hypercube.

To permit retrieval of collections of particles that are similar to each other with respect to continuous or non-discrete features, barcode sequences are mutated at a small number of carefully selected sites within the sequence. A restricted set of mutated variant barcode sequences are represented in a graph G, such as, but not limited to, a hypercube graph. The mutation sites are selected so that the graph G faithfully represents the binding affinity between the barcodes and the complementary sequences to the barcodes that are to be used as probes. The similarity space of the continuous feature is also represented in a graph H, which is subsequently embedded isometrically into the graph G. For certain simple graphs H, an exact isometric embedding may be found using polynomial time algorithms. For arbitrary, complex graphs H, the isometric embedding may be found by first performing dimensional reduction on the corresponding metric space represented by H. The dimensional reduction may be performed using any standard technique that attempts to preserve distance during the transformation. The lower-dimensional space may then be discretized to approximate an isometric embedding into G. Examples of finding an isometric embedding both when H is simple and complex are shown in FIGS. 27 and 28.

The term “hypercube” as used herein, refers to an extrapolation of a cube or square to n dimensions. For example, a 4th dimensional hypercube is called a tesseract. Therefore, an n-dimensional hypercube is also known as an n-cube. It is best drawn and represented in non-Euclidean geometry.

Therefore, in some forms, the methods for retrieval of encapsulated sequence storage objects target one or more populations of interest for retrieval from a pool of populations based on approximate similarity-based retrieval of the target population. The methods retrieve sequence storage objects of interest from a pool of sequence storage objects, wherein the sequence storage objects of interest include molecular tags corresponding to one or more characteristics associated with an arbitrarily complex similarity metric.

### i. Barcode Design by Isometric Embedding

In some forms, molecular “barcode” tags associated with the sequence storage objects are nucleic acid sequences that include or encode a sequence associated with one or more characteristics determined by isometric embedding, whereby the isometric embedding corresponds directly to an assignment of barcodes to each particle that permits similarity-based retrieval. Therefore, in some forms, the methods include one or more steps for designing the sequences of molecular “barcode” tags by isometric embedding.

In some forms, the methods design the tags by representing a simple similarity metric as a cyclic graph with “n” nodes that may be isometrically embedded exactly into a 4-dimensional hypercube graph. In an exemplary form, a simple similarity metric is represented in a cyclic graph with 8 nodes that may be isometrically embedded exactly into a 4-dimensional hypercube graph, as depicted in FIG. 27.

A schematic of an exemplary barcode sequence design process that enables approximate similarity-based retrieval with respect to a feature with an arbitrarily complex similarity metric is set forth in FIG. 28. In an exemplary form, the feature similarity space is simplified using standard dimensional reduction to reduce it to a small number of dimensions. These dimensions are then approximated further by binning, after which they can be embedded directly into a hypercube graph whose nodes represent mutational variants of a set of barcodes.

In an exemplary method, the process begins with a complex similarity metric derived from, for example, 4187 SARS-CoV2 genomes whose pairwise genetic similarity was computed. This similarity metric is reduced to 18 dimensions using multidimensional scaling (MDS); for visualization purposes, the number of dimensions was reduced further to 2 dimensions before plotting. After binning, linear regression showed a strong correlation between the original similarity metric and the final distance in a 54-dimensional hypercube embedding. The hypercube embedding corresponded directly to an assignment of 6 barcode sequences to each node in the original feature space.

Therefore, in some forms, methods for designing molecular barcode tags correlated with two or more similar features include

(a) determining a low-dimensional feature similarity metric for the two or more similar features by simplifying the feature similarity space of the two or more similar features;

(b) embedding the simplified features directly into a hypercube graph, e.g., wherein the similarity metric is correlated with distance in the hypercube embedding to provide correspondingly differing barcode sequences; and

(c) generating the barcode sequence tags.

(a) Simplifying the Feature Similarity Space

In some forms, the methods for designing molecular barcode tags include one or more steps for determining a similarity metric for a complex similarity metric for the two or more features. Exemplary methods for providing a complex similarity amongst a pool of two or more samples include determining a feature similarity metric, such as sequence identity, etc. between each of the members of the pool. In exemplary forms, a population includes a library of distinct species, such as a library of genomic sequences, for example, a library of viral genomic sequences. Similarity between the members of a population of viral genomic sequences can be assessed, for example, by sequence identity to each other.

In some forms, prior to mapping the features to which the feature tags correspond, the dimensionality of the features to which the feature tags correspond is reduced.

Therefore, in some forms, the methods for designing molecular barcode tags include one or more steps for simplifying the feature similarity space by dimensional reduction to provide a feature similarity metric. In some forms, simplifying the feature similarity space includes using standard dimensional reduction. In particular forms, the similarity metric is reduced using multidimensional scaling (MDS). Typically, the feature similarity space is reduced to a small number of dimensions, such as from about 2 to about 20 dimensions, inclusive. Therefore, in some forms, the similarity encoded feature tags of the set of feature tags are similarity encoded by reducing the dimensionality of the features to which the feature tags correspond.

(b) Embedding Directly into a Hypercube Graph

In some forms, the dimensionality-reduced features are mapped to the hypercube based on the similarity of the dimensionality-reduced features.

Therefore, in some forms, the methods include one or more steps for further approximating the dimensions by binning and embedding directly into a “n” dimensional hypercube graph whose nodes represent mutational variants of a set of barcodes, where “n” is an integer less than or equal to the number of features to which the feature tags correspond, and where “n” is a factor of the number of features to which the feature tags correspond. In some forms, the methods map the dimensionality-reduced features to an n-dimensional hypercube based on the similarity of the dimensionality-reduced features, wherein n is an integer less than or equal to the number of features to which the feature tags correspond, wherein n is a factor of the number of features to which the feature tags correspond. In some forms, the methods implement a computer system to complete one or more of the steps. For example, in some forms, the mapping is implemented using a computer.

In some forms, the quality of this mapping may be assessed by calculating a correlation between the distance in the original similarity metric and the distance in the n-dimensional hypercube after embedding. In some forms, linear regression modelling may be used to calculate this correlation. A high correlation (i.e., close to 1) indicates that the mapping preserves well the similarities between features as described by the original similarity metric. In some forms, the correlating includes linear regression modelling. Preferably, the hypercube embedding corresponds directly to an assignment of barcode sequences to each node in the original feature space. In some forms, the number of edges of the hypercube between the nodes to which any two of the mapped features are mapped is proportional to the similarity of the two features.

(c) Generating Molecular Barcode Tags

In some forms, the methods include one or more steps for generating the molecular barcode tags according to an assignment of barcode sequences to nodes in the n-dimensional hypercube. A restricted set of barcode sequence variants is generated by mutating at a small number of sites, such that the binding affinities between barcodes and their complements (i.e. probes) are represented accurately in an n-dimensional hypercube.

This hypercube determines a barcode sequence for each node in the n-dimensional hypercube of (b). Using the mapping determined in (a) and (b), this determines a barcode sequence for each node in the original feature space. The barcode sequences are then associated with the corresponding sequence controlled polymer(s) to produce a tagged sequence storage object.

3. Boolean Logic

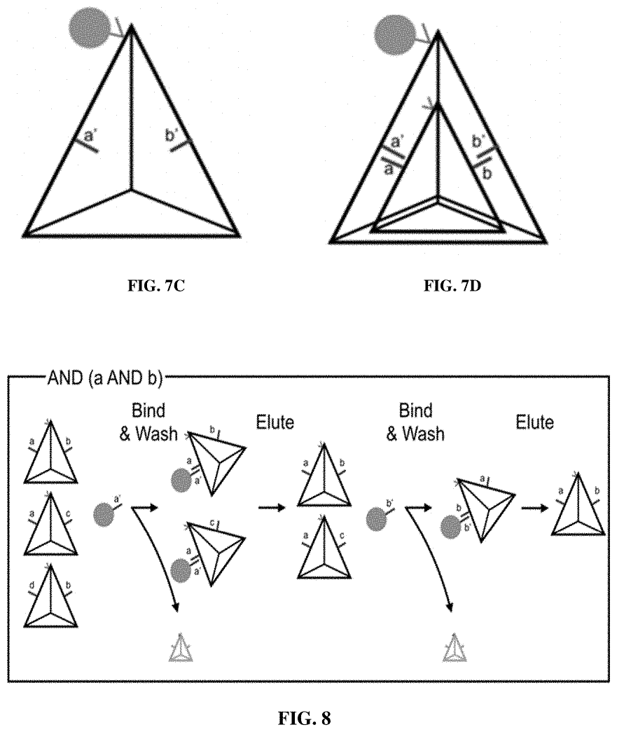

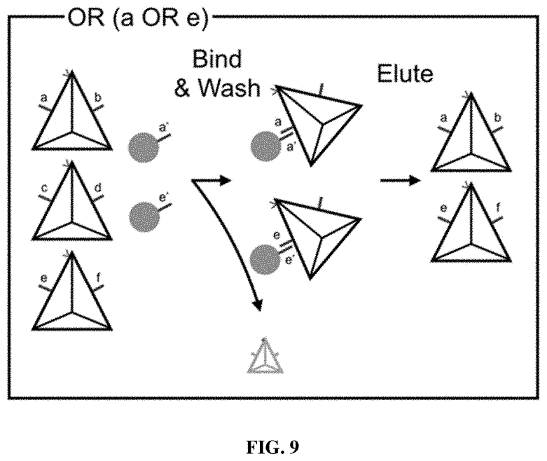

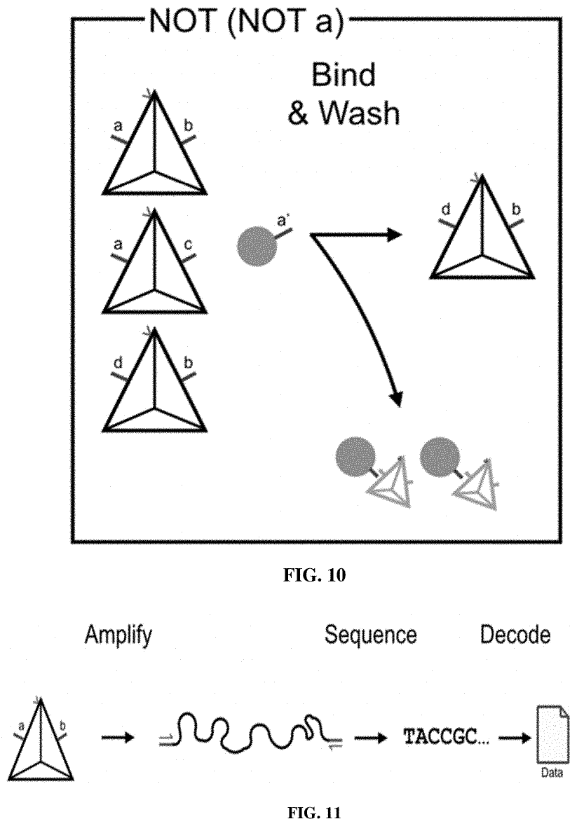

In some forms, Boolean logic of AND, OR, and NOT are applied to SSOs using the tag overhang sequences as described in FIGS. 8A-8E, FIGS. 9A-9C, and FIGS. 10A-10B. These logic applications are complementary. In some forms, these logic applications are applied once. In other forms, the same logic application is applied multiple times, for example, 2, 3, 4, 5, 6, 7, 8, 9, 10, 20, 30, 40, 50, 100 times, or more than 100 times. An exemplary multiple applications of the same logic is a AND b AND c AND d AND e, etc. In some forms, these logic applications are used in any desired order or combination to generate large sets of logical computations. An exemplary combination is a AND b, followed by NOT c. In some forms, these logic applications are used in any desired order or combination multiple times, for example, 2, 3, 4, 5, 6, 7, 8, 9, 10, 20, 30, 40, 50, 100 times, or more than 100 times.

i. AND Logic

In some forms, AND logic is applied in the selection and purification of a SSO with two or more overhang tag sequences (FIGS. 8A-8E). A SSO or set of SSOs is purified from a pool of SSOs when the targeted SSOs are able to be separated using AND logic. For example, a SSO or set of SSOs of interest are purified in multiple rounds, first using a capture support specific to one overhang of interest (i.e., capturing all SSOs with the overhang sequence a). Unbound NSOs are then washed away, leaving the bound SSOs attached to the capture support, as described in FIGS. 5A-5C. Captured SSOs are then released from the support by changing the pH, lowering the salt, increasing the temperature, toe-hold strand displacement, enzymatic release by restriction nucleases, nickases, helicases, resolvases, UV/light sensitive linkers, or any combination thereof. The pool of released SSOs from this first round are then applied to a second round of purification with a second, distinct set of capture sequences bound to a support. The SSOs are then captured on the second capture support with a distinct capture sequence (i.e., capturing all SSOs of the released pool having an overhang sequence b) and unbound SSOs are washed away as in FIGS. 6A-6C. The bound SSO(s) are then released from the support by changing the pH, lowering the salt, increasing the temperature, toe-hold strand displacement, enzymatic release by restriction nucleases, nickases, helicases, resolvases, with UV or light, or any combination thereof. This yields SSOs with overhang sequences a AND b. In some forms, this AND logic purification process is repeated twice, three times, four times, five time, up to ten times, or more than ten times. In some forms, this AND logic purification process is repeated for the number of instances of tags on a given object (2×(number of staples)).

F. Retrieval of Sequence-Controlled Polymers from SSOs

The methods include retrieving the sequence-controlled polymers stored within sequence-controlled polymers objects. For example, in some forms the methods include retrieving the nucleic acid nanostructures.

### 1. Retrieval of Sequence-Controlled Polymers from NSOs

In some forms, methods for dissociation of NSOs to their single-strand components include denaturation of NSOs. NSOs can be denatured by changes in pH, or temperature. In an exemplary form, NSOs are denatured by melting (FIGS. 11A-11D). The released single-strand scaffold is purified and amplified by virtue of master primer sequences flanking the DNA sequence. The nucleotide sequence is read out via any known sequencing methods. In some forms, PCR is used to amplify the final selected message. In some forms PCR is achieved using a set of primers that are specific to the NSO of interest. In some forms, PCR is carried out using a set of “master primers” that are tested to be orthogonal to the sequences. Typically, the object pool is specifically selected to narrow down the pool to only messages that satisfy the user request. When all the sequence-controlled polymers within NSOs is surrounded by a single set of master primers, only a single PCR reaction is necessary in the workflow. In some forms, barcode sequences are generated on the surface of nanoparticle and/or microparticle scaffolds using a DNA synthesizer. The barcode-modified scaffolds capture the requested NSOs from the object pool. In some forms, barcode sequences generated on chip arrays capture the requested NSOs from the object pool for retrieval and subsequent PCR amplification.

### i. Sequencing Methods

Any known DNA sequencing methods can be used. In some forms, the nucleotide sequence is read out via sequencing methods including Sanger sequencing (Sanger F et al., Proc. Natl. Acad. Sci. U.S.A. 74 (12): 5463-7(1977)).

In some forms, the nucleotide sequence is read out via Maxam & Gilbert sequencing (Maxam A M et al., Proc. Nat. Acad. Sci. USA 74,560-564 (1977)), or any other chemical methods. In other forms, sequencing is done by PYROSEQUENCING™. In further forms, the nucleotide sequence is read out by single molecule sequencing using exonuclease.

In some forms, sequencing is done by next generation sequencing. Some exemplary technologies include ILLUMINA®, Roche 454 sequencing, Ion torrent: Proton/PGM sequencing, SOLiD sequencing. Some exemplary commercial providers of next generation sequencing are Pacific Biosciences, ILLUMINA®, Oxford Nanopore Technologies.

### ii. Error Correction

DNA synthesis generates errors in the nucleotide sequence, with the error rates on the order of 1% per nucleotide. Furthermore, long-term storage of NSOs will compromise data integrity. In some forms, errors are reduced by increase data redundancy, by means of storing NSOs, or by replicating NSOs periodically.

### iii. Data Redundancy

A key aspect of DNA storage is to devise appropriate schemes that tolerate errors by adding redundancy. In some forms, errors are tolerated by adding redundancy at the stage of encoding. For example, the encoding proposed by Goldman et al., where the input DNA nucleotides are split into overlapping segments to provide multiple fold redundancy for each segment (Goldman N et al., Nature, 494:77-80 (2013)). In some forms, the encoding redundancy is incorporated as proposed by Bornholt J et al. (Bornholt, J et al., 21th ACM International Conference on Architectural Support for Programming Languages and Operating Systems. (2016)) using exclusive, or of two payloads to form a third strand.

### iv. Replication of NSOs

For long term storage of sequence-controlled polymers via NSOs, deamination is the highest source of information loss in ancient DNA and has the lowest energy barrier (Zhirnov V et al., Nat Mater. 23; 15(4):366-70 (2016)). To combat information loss in practical storage or storage systems, error-correction codes are widely used (Kim C et al., IEEE Trans. Consum. Electron. 61, 206-214 (2015)). Fortunately, nucleic acid is easy to copy, which decreases the ECC overhead and thus makes error correction a primary factor for data integrity. In some forms, nucleic acids are replicated into numerous physical copies of itself with high fidelity and low cost.

### III. Databases

The methods can include the creation of databases. Databases can be used to enable or assist subsequent analysis of the same or different samples. For example, databases can be used to assist the analysis of one or more similar types of samples having similar or different levels of heterogeneity.

For example, the methods can include a step of developing a database of sequence-controlled polymers. Databases can be initiated, developed and maintained in any format known in the art, for example by employing a data system such as a digital computer. In some forms, sequence-controlled polymers for populating a database can be accumulated by including a sufficiently large number of samples, for example, by creating a library of nucleic acid nanostructures, and/or encapsulated nucleic acid units.

Typically, databases include at least two different pieces of data, such as sequences or tags that can be used to identify sequence-controlled polymers, or subsets of sequence-controlled polymers. In some forms, databases include nucleic acid sequences and/or corresponding barcodes for each sequence-controlled polymer object in a pool, for example, corresponding to each SSO in a pool, or a library of SSOs. In some forms, each tag or barcode in a database corresponds to one or more sequences or other features of sequence-controlled polymers. Databases populated with binary barcodes depicting the sequences of different sequence-controlled polymers, such as a library of SSOs produced according to the described methods, can be developed. Databases can store binary sequence barcodes corresponding to one or more different pools of objects. For example, a database can include of tens, hundreds, thousands of more non-contiguous nucleic acid sequences.

In some forms, the generation of a multiply-addressed pool of SSOs will act as a database for the long-term storage of sequence-controlled polymers. Multiple indices on features will allow for highly specific extraction of sequence-controlled polymers based on features used. Therefore, in some forms, the database is searched using features based on nucleic acid sequences complementary to the tags of the SSOs. In some forms, the tag is encoded by a known scheme such that no external database is needed to extract SSOs based on metadata. This direct conversion of metadata to capture sequence can be used to mine sequence-controlled polymers contained within the solution-database of SSOs as deeply as allowed by the number of allowed tags on a given geometry. Common database queries can be used against a system, such as PUT, GET, Delete, AND, and OR. Thus, a database of all sequence-controlled polymers of a SSO can be indexed with various features of the sequence-controlled polymers. A particular feature can then be extracted out after the pool of all objects has been probed to capture the specific feature of interest. Using associative storage would allow for specific aggregation of records satisfying a set of criteria generated by the user and when given the proper signal. For example, all sequence-controlled polymers from a given species could be associated to a superstructure.

### IV. Compositions

The compositions described below include materials, compounds, and components that can be used for the disclosed methods. Various exemplary combinations, subsets, interactions, groups, etc. of these materials are described in more detail above. However, it will be appreciated that each of the other various individual and collective combinations and permutations of these compounds that are not described in detail are nonetheless specifically contemplated and disclosed herein. For example, if one or more nucleic acid nanostructures are described and a number of substitutions of one or more of the structural or sequence parameters are discussed, each and every combination and permutation of the structural or sequence parameters possible are specifically contemplated unless specifically indicated to the contrary.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

A. Nucleic Acid Storage Objects

1. Nucleic Acid Samples

Nucleic acids for use in the described methods can be synthesized or natural nucleic acids. In some forms, the nucleic acid sequences are not naturally occurring nucleic acid sequences. In some forms, the nucleic acid sequences are synthetic nucleic acid sequences. In some forms, the nucleic acid nanostructures are not genomic nucleic acid of a virus. In some forms, the nucleic acid nanostructures are virus-like particles.

Numerous other sources of nucleic acid samples are known or can be developed and any can be used with the described method. In some forms, nucleic acids used in the described methods are naturally occurring nucleic acids. Examples of suitable nucleic acid samples for use in the described methods include genomic samples, RNA samples, cDNA samples, nucleic acid libraries (including cDNA and genomic libraries), whole cell samples, environmental samples, culture samples, tissue samples, bodily fluids, and biopsy samples.

Nucleic acid fragments are segments of larger nucleic molecules. Nucleic acid fragments, as used in the described method, generally refer to nucleic acid molecules that have been cleaved. A nucleic acid sample that has been incubated with a nucleic acid cleaving reagent is referred to as a digested sample. A nucleic acid sample that has been digested using a restriction enzyme is referred to as a digested sample.

In certain forms, the nucleic acid sample is a fragment or part of genomic DNA, such as human genomic DNA. Human genomic DNA is available from multiple commercial sources (e.g., Coriell #NA23248). Therefore, nucleic acid samples can be genomic DNA, such as human genomic DNA, or any digested or cleaved sample thereof. Generally, an amount of nucleic acids between 375 bp and 1,000,000 bp is used per nucleic acid nanostructure.

2. Nucleic Acid Nanostructures

The basic technique for creating nucleic acid (e.g., DNA) origami of various shapes involves folding a long single stranded polynucleotide, referred to as a “scaffold strand,” into a desired shape or structure using a number of small “staple strands” as glue to hold the scaffold in place. Several variants of geometries can be used for construction of NSOs. For example, in some forms, NSOs from purely shorter single stranded staples can be assembled, or NSOs including purely a single stranded scaffold folded onto itself, any of which can take on diverse geometries/architectures including wireframe or bricklike objects.

### i. Staple Strands

The number of staple strands will depend upon the size of the scaffold strand and the complexity of the shape or structure. For example, for relatively short scaffold strands (e.g., about 50 to 1,500 base in length) and/or simple structures the number of staple strands are small (e.g., about 5, 10, 50 or more). For longer scaffold strands (e.g., greater than 1,500 bases) and/or more complex structures, the number of staple strands are several hundred to thousands (e.g., 50, 100, 300, 600, 1,000 or more helper strands).

Typically, Staple strands include between 10 and 600 nucleotides, for example, 14-600 nucleotides.

In scaffolded DNA origami, a long single-stranded DNA is associated with complementary short single-stranded oligonucleotides that bring two distant sequence-space parts of the long strand together to fold into a defined shape. Historically, folding of DNA nanostructures has relied on tedious per-object design without generalized scaffold sequence choice.

A robust computational-experimental approach is used to generate DNA-based wireframe polyhedral structures of arbitrary scaffold sequence, symmetry and size. These DNA origami objects have several important properties that render them useful for DNA-based storage, including 1) arbitrary numbers of faces or edges that are programmed to present outward-facing ssDNA tags that act as either handles to physically associate with other storage blocks or act as barcodes on these storage blocks for bead-based or other physical extraction/purification; 2) they do not associate or aggregate with one another non-specifically because they have an absence of free duplex ends, unlike brick-like origami; 3) they are porous so that small molecules and other singles-stranded nucleic acids as well as restriction enzymes and polymerases may diffusive through these storage blocks even when assembled into supramolecular storage blocks; 4) they remain stably folded under moderate ionic strengths; 5) unlike unpaired single-stranded DNA that associates non-specifically with itself and other strands of partial base complementarity, these DNA nanostructure origami sequester single-stranded DNA in a tightly associated, stable form that renders biochemical purification and transport practical.

### ii. Geometric Shapes of NSOs

NSOs are nucleic acid assemblies of any arbitrary geometric shapes. NSOs can be of two-dimensional shapes, for example plates, or any other 2-D shape of arbitrary sizes and shapes. In some forms, the NSOs are simple DX-tiles, with two DNA duplexes connected by staples. DNA double crossover (DX) motifs are examples of small tiles (˜4 nm×˜16 nm) that have been programmed to produce 2D crystals (Winfree E et al., Nature. 394:539-544(1998)); often these tiles contain pattern-forming features when more than a single tile constitutes the crystallographic repeat. In some forms, NSOs are 2-D crystalline arrays by parallel double helical domains with sticky ends on each connection site (Winfree E et al., Nature. 6; 394(6693):539-44 (1998)). In some forms, NSOs are 2-D crystalline arrays by parallel double helical domains, held together by crossovers (Rothemund P W K et al., PLoS Biol. 2:2041-2053 (2004)). In some forms, NSOs are 2-D crystalline arrays by an origami tile whose helix axes propagate in orthogonal directions (Yan H et al., Science. 301:1882-1884 (2003)).

In some forms, NSOs are wireframe nucleic acid (e.g., DNA) assemblies of a uniform polyhedron that has regular polygons as faces and is isogonal. In some forms, NSOs are wireframe nucleic acid (e.g., DNA) assemblies of an irregular polyhedron that has unequal polygons as faces. In some forms, NSOs are wireframe nucleic acid assemblies of a convex polyhedron. In some further forms, NSOs are wireframe nucleic acid assemblies of a concave polyhedron. In some further forms, NSOs are brick-like square or honeycomb lattices of nucleic acid duplexes in cubes, rods, ribbons or other rectilinear geometries. The corrugated ends of these structures are used to form complementary shapes that can self-assemble via non-specific base-stacking. Some exemplary superstructures of NSOs include Platonic, Archimedean, Johnson, Catalan, and other polyhedral. In some forms, Platonic polyhedron are with multiple faces, for example, 4 face (tetrahedron), 6 faces (cube or hexahedron), 8 face (octahedron), 12 faces (dodecahedron), 20 faces (icosahedron). In some forms, NSOs are toroidal polyhedra and other geometries with holes. In some forms, NSOs are wireframe nucleic acid assemblies of any arbitrary geometric shapes. In some forms, NSOs are wireframe nucleic acid assemblies of non-spherical topologies. Some exemplary topologies include nested cube, nested octahedron, torus, and double torus.

In preferred forms, a set of tags to be associated with the sequence-controlled polymers on a NSO are selected and then encoded into a nucleic acid (DNA or locked nucleic acids or RNA, etc.) sequence using a conversion method of the user's choice. In some forms, it also includes a mechanism of direct conversion from, including but not limited to strings, integers, dates, events, genres, metadata, participants, or authors. In further forms, this additionally includes direct sequence selection, with the user keeping an external library of addresses.

B. Sequence-Controlled Polymer Encapsulation

Single- and/or double-stranded DNA or any other sequence-controlled polymer can be encapsulated to generate SSOs. These encapsulated acid sequence-controlled polymer units can also have one or more surface-based molecular identifier (feature tag) for physical selection and manipulation. Typically, the encapsulated acid sequence-controlled polymer units are designed for reversibility and recovery of the intact encapsulated sequence-controlled polymer, thus allowing for sequencing and readout of the sequence-controlled polymer.

The encapsulated storage objects typically include one or more feature tags coupled to the exterior of the coating. Feature tags can be are directly or indirectly. Feature tag-functionalized particles are pooled and stored for downstream object selection and polymer retrieval. In further forms, the feature tags on the surface of the SSO-containing particles are used to select objects using a complementary strand to isolate the desired object from the object pool. The SSOs are released from the particles using a buffered oxide etch. The SSOs can then be processed for decoding and readout.

1. Sequence-Controlled Polymers to be Encapsulated

Sequence-controlled polymers to be encapsulated can take any arbitrary form, for example, a linear or branched polypeptide, a linear or branched carbohydrate, a protein, a glycosylated polypeptide, a linear nucleic acid sequence, a two-dimensional nucleic acid object or a three-dimensional nucleic acid object. In some forms, the linear nucleic acid are base-paired double stranded. In other forms, the linear nucleic acids include a long continuous single-stranded nucleic acid polymer or many such polymers. In further forms, sequence-controlled polymers encapsulated within the same particle are a mixture of any one or more of a linear, or non-linear single or double stranded nucleic acid molecule, a polypeptide, a carbohydrate, a protein, or a glycosylated polypeptide. For example, is some forms, one or more single-stranded nucleic acids and one or more scaffolded nucleic acid nanostructure are encapsulated within the same particle.

2. Encapsulating Agents

In some forms, sequence-controlled polymers are packaged into discrete SSOs via encapsulation. For example, in some forms, nucleic acids are packaged into discrete NSOs via encapsulation. Suitable encapsulating agents include gel-based beads, protein viral packages, micelles, mineralized structures, siliconized structures, or polymer packaging.

In some forms, the encapsulating agents are viral capsids or a functional part, derivative and/or analogue thereof. In some forms, the NSOs are viral like particles, with nucleic acid content enveloped by protein content on the surface. Viral capsids can be derived from retroviruses, human papilloma viruses, M13 viruses, adeno viruses adeno-associated viruses, for example, adenovirus 16. In preferred forms, viral capsids used for encapsulating NSOs do not interfere with the overhang tags i.e. overhang tags are accessible for purification purposes.

In some forms, the encapsulating agents are lipids forming micelles, or liposomes surrounding the nucleic acid. In some forms, micelles, or liposomes are formed from one or more lipids, which can be neutral, anionic, or cationic at physiologic pH. Suitable neutral and anionic lipids include, but are not limited to, sterols and lipids such as cholesterol, phospholipids, lysolipids, lysophospholipids, sphingolipids or pegylated lipids. Neutral and anionic lipids include, but are not limited to, phosphatidylcholine (PC) (such as egg PC, soy PC), including, but not limited to, 1,2-diacyl-glycero-3-phosphocholines; phosphatidylserine (PS), phosphatidylglycerol, phosphatidylinositol (PI); glycolipids; sphingophospholipids such as sphingomyelin and sphingoglycolipids (also known as 1-ceramidyl glucosides) such as ceramide galactopyranoside, gangliosides and cerebrosides; fatty acids, sterols, containing a carboxylic acid group for example, cholesterol; 1,2-diacyl-sn-glycero-3-phosphoethanolamine, including, but not limited to, 1,2-dioleylphosphoethanolamine (DOPE), 1,2-dihexadecylphosphoethanolamine (DHPE), 1,2-distearoylphosphatidylcholine (DSPC), 1,2-dipalmitoyl phosphatidylcholine (DPPC), and 1,2-dimyristoylphosphatidylcholine (DMPC). The lipids can also include various natural (e.g., tissue derived L-(α-phosphatidyl: egg yolk, heart, brain, liver, soybean) and/or synthetic (e.g., saturated and unsaturated 1,2-diacyl-sn-glycero-3-phosphocholines, 1-acyl-2-acyl-sn-glycero-3-phosphocholines, 1,2-diheptanoyl-SN-glycero-3-phosphocholine) derivatives of the lipids.

Suitable cationic lipids in the micelles, or the liposomes include, but are not limited to, N-[1-(2,3-dioleoyloxy)propyl]-N,N,N-trimethyl ammonium salts, also references as TAP lipids, for example methylsulfate salt. Suitable TAP lipids include, but are not limited to, DOTAP (dioleoyl-), DMTAP (dimyristoyl-), DPTAP (dipalmitoyl-), and DSTAP (distearoyl-). Suitable cationic lipids in the liposomes include, but are not limited to, dimethyldioctadecyl ammonium bromide (DDAB), 1,2-diacyloxy-3-trimethylammonium propanes, N-[1-(2,3-dioloyloxy)propyl]-N,N-dimethyl amine (DODAP), 1,2-diacyloxy-3-dimethylammonium propanes, N-[1-(2,3-dioleyloxy)propyl]-N,N,N-trimethylammonium chloride (DOTMA), 1,2-dialkyloxy-3-dimethylammonium propanes, dioctadecylamidoglycylspermine (DOGS), 3-[N-(N′,N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA), β-alanyl cholesterol, cetyl trimethyl ammonium bromide (CTAB), diC₁₄-amidine, N-ferf-butyl-N′-tetradecyl-3-tetradecylamino-propionamidine, N-(alpha-trimethylammonioacetyl)didodecyl-D-glutamate chloride (TMAG), ditetradecanoyl-N-(trimethylammonio-acetyl)diethanolamine chloride, 1,3-dioleoyloxy-2-(6-carboxy-spermyl)-propylamide (DOSPER), and N, N, N′, N′-tetramethyl-, N′-bis(2-hydroxylethyl)-2,3-dioleoyloxy-1,4-butanediammonium iodide. In one form, the cationic lipids can be 1-[2-(acyloxy)ethyl]2-alkyl(alkenyl)-3-(2-hydroxyethyl)-imidazolinium chloride derivatives, for example, 1-[2-(9(Z)-octadecenoyloxy)ethyl]-2-(8(Z)-heptadecenyl-3-(2-hydroxyethyl)imidazolinium chloride (DOTIM), and 1-[2-(hexadecanoyloxy)ethyl]-2-pentadecyl-3-(2-hydroxyethyl)imidazolinium chloride (DPTIM). In one form, the cationic lipids can be 2,3-dialkyloxypropyl quaternary ammonium compound derivatives containing a hydroxyalkyl moiety on the quaternary amine, for example, 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimetyl-hydroxypropyl ammonium bromide (DORIE-HP), 1,2-dioleyl-oxy-propyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), and 1,2-disteryloxypropyl-3-dimethyl-hydroxyethyl ammonium bromide (DSRIE).

The lipids may be formed from a combination of more than one lipid, for example, a charged lipid may be combined with a lipid that is non-ionic or uncharged at physiological pH. Non-ionic lipids include, but are not limited to, cholesterol and DOPE (1,2-dioleolylglyceryl phosphatidylethanolamine).

In some forms, the encapsulating agents are natural or synthetic polymers. Representative natural polymers are proteins, such as zein, serum albumin, gelatin, collagen, and polysaccharides, such as cellulose, dextrans, and alginic acid. Representative synthetic polymers include polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes, alkyl cellulose, hydroxyalkyl celluloses, cellulose ethers, cellulose esters, nitrocelluloses, polymers of acrylic and methacrylic esters, poly[lactide-co-glycolide], polyanhydrides, polyorthoestersblends and copolymers thereof. Specific examples of these polymers include cellulose acetate, cellulose propionate, cellulose acetate butyrate, cellulose acetate phthalate, carboxymethyl cellulose, cellulose triacetate, cellulose sulphate, poly(methyl methacrylate), (poly(ethyl methacrylate), poly(butyl methacrylate), Poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate), polyethylene, polypropylene, poly(ethylene glycol), poly(ethylene oxide), poly(ethylene terephthalate), poly(vinyl alcohols), poly(vinyl acetate), poly(vinyl chloride), polystyrene and polyvinylpyrrolidone, polyurethane, polylactides, poly(butyric acid), poly(valeric acid), poly[lactide-co-glycolide], polyanhydrides, polyorthoesters, poly(fumaric acid), and poly(maleic acid).

In some forms, the encapsulating agents are mineralized, for example, calcium phosphate mineralization of alginate beads, or polysaccharides. In other forms, the encapsulating agents are siliconized. In one form, the nucleic acid is packaged in a mineral structure, but has on its surface single-stranded nucleic acids that act as the address used for association with other NSOs, or selection by Boolean logic.

In some forms, the encapsulating agents are metal oxide particles. Exemplary metal oxide encapsulating agents include silicon dioxide (SiO₂) and titanium dioxide (TiO₂), that can be mesoporous, compact, or structured. In some forms, the DNA is adsorbed on the surface of a modified metal oxide particle then coated with polyelectrolytes, for example poly(diallyldimethylammonium chloride), poly(acrylamide-co-diallyldimethylammonium chloride), and poly(allylamine hydrochloride).

3. Feature Tags

In some forms, the feature tags are directly synthesized on to the encapsulated storage objects. In one form, NSO-containing particles that have surfaces coated with 9-O-dimethoxytrityl (DMT)-triethylene glycol,1-[(2-cyanoethyl)-(N, N-diisopropyl)]-phosphoramidite. When a DNA synthesizer is used to generate the feature tags, modified silica particles are used directly as the solid-phase support for the DNA synthesizer. In other forms, the feature tags are synthesized separately and are attached on the surface of NSO-containing particles using chemical conjugation. For example, in some forms, feature tags are conjugated to storage objects wherein the conjugation chemistry involves biotin-avidin recognition pairs, N-hydroxysuccinimide (NHS) coupling, 1-ethyl-3-(3-dimethylaminopropyl) carbodiimide (EDC) coupling, succinimidyl 4-(N-maleimidomethyl)cyclohexane-1-carboxylate (SMCC)-mediated coupling, sulfo-SMCC coupling, copper-catalyzed azide-alkyne cycloaddition (CuAAC), strain-promoted azide-alkyne cycloaddition (SPAAC), or combinations of these. Feature tag-functionalized particles are pooled and stored for downstream object selection and polymer retrieval. In further forms, the feature tags on the surface of the SSO-containing silica particles are used to select objects using a complementary strand to isolate the desired data from the object pool. The SSOs are released from the silica particles using a buffered oxide etch. The SSOs can then be processed for decoding and readout.

In addition to nucleic acid overhangs, other purification tags can be incorporated into the overhang nucleic acid sequence in any SSOs for purification (i.e. object retrieval). In some forms, the overhang contains one or more purification tags. In some forms, the overhang contains purification tags for affinity purification. In some forms, the overhang contains one or more sites for conjugation to a nucleic acid, no non-nucleic acid molecule. For example, the overhang tag can be conjugated to a protein, or non-protein molecule, for example, to enable affinity-binding of the SSOs. Exemplary proteins for conjugating to overhang tags include biotin and antibodies, or antigen-binding fragments of antibodies. Purification of antibody-tagged SSOs can be achieved, for example, via interactions with antigens, and or protein A, G, A/G or L.

Further exemplary affinity tags are peptides, nucleic acids, lipids, saccharides, or polysaccharides. For example, overhang contains saccharides such as mannose molecules, then mannose-binding lectin can be used for selectively retrieve mannose-containing SSOs, and vice versa. Other overhang tags allow further interaction with other affinity tags, for example, any specific interaction with magnetic particles allows purification by magnetic interactions.

4. Nucleic Acid Overhang Tag

In some forms, the overhang sequences are between 4 and 60 nucleotides, depending on user preference and downstream purification techniques. In preferred forms, the overhang sequences are between 4 and 25 nucleotides. In some forms, the overhang sequences contain 4, 5, 6, 7, 8, 9, 10, 11, 12, 13, 14, 15, 16, 17, 18, 19, 20, 21, 22, 23, 24, 25, 30, 35, 40, 45, 50 nucleotides in length.

In some forms, these overhang tag sequences are placed on the 5′ end of any of the staples used to generate a wireframe nucleic acid. In other forms, these overhang tag sequences are placed on the 3′ end of any of the staples used to generate a wireframe nucleic acid.

In some forms, overhang tag sequences contain metadata for the scaffolded nucleic acid, or the encapsulated nucleic acid. For example, overhang tag sequences have address(es) for locating a particular sequence-controlled polymer. In some further forms, each overhang tag contains a plurality of functional elements such as addresses, as well as region(s) for hybridizing to other overhang tag sequences, or to bridging strands. These tag sequences added to the staple sequences at user-defined locations, with the untagged staple strands are then synthesized individually or as a pool directly using any known methods.

5. Modifications to Nucleotides

In some forms, one or more of the nucleotides of the feature tags of SSOs are modified nucleotides. In some forms, one or more of the nucleotides of the scaffolded nucleic acid sequences of NSOs are modified nucleotides. In some forms, the nucleotides of the encapsulated nucleic acid sequences of NSOs are modified. In some forms, one or more of the nucleotides of the nucleic acid staple sequences are modified nucleotides. In some forms, the nucleotides of the DNA tag sequences are modified for further diversification of addresses associated with SSOs. Examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, and (acp3)w, 2,6-diaminopurine. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Locked nucleic acid (LNA) is a family of conformationally locked nucleotide analogues which, amongst other benefits, imposes truly unprecedented affinity and very high nuclease resistance to DNA and RNA oligonucleotides (Wahlestedt C, et al., Proc. Natl Acad. Sci. USA, 975633-5638 (2000); Braasch, D A, et al., Chem. Biol. 81-7 (2001); Kurreck J, et al., Nucleic acids Res. 301911-1918 (2002)). In some forms, the scaffolded DNAs are synthetic RNA-like high affinity nucleotide analogue, locked nucleic acids. In some forms, the staple strands are synthetic locked nucleic acids.

Peptide nucleic acid (PNA) is a nucleic acid analog in which the sugar phosphate backbone of natural nucleic acid has been replaced by a synthetic peptide backbone usually formed from N-(2-amino-ethyl)-glycine units, resulting in an achiral and uncharged mimic (Nielsen, et al., Science 254, 1497-1500 (1991)). It is chemically stable and resistant to hydrolytic (enzymatic) cleavage. In some forms, the scaffolded DNAs are PNAs. In some forms, the staple strands are PNAs.

In some forms, a combination of PNAs, DNAs, and/or LNAs is used for the nucleic acids in an NSO. In other forms, a combination of PNAs, DNAs, and/or LNAs is used for the staple strands, overhang sequences, or any nucleic acid component of the SSOs.

### V. Devices, Data Structures and Computer Control

Described are data structures used in, generated by, or generated from, the described method. Data structures generally are any form of data, information, and/or objects collected, organized, stored, and/or embodied in a composition or medium. For example, the nucleotide sequence associated with a nucleic acid nanostructure labeled with a specific sequence tag, or set of sequences stored in electronic form, such as in RAM or on a storage disk, is a type of data structure. The described method, or any part thereof or preparation therefor, can be controlled, managed, or otherwise assisted by computer control. Such computer control can be accomplished by a computer controlled process or method, can use and/or generate data structures, and can use a computer program. Such computer control, computer controlled processes, data structures, and computer programs are contemplated and should be understood to be described herein.

The methods and general approach towards molecular data storage and computation can be carried out using a computer-based system. In some forms, one or all of the method steps are carried out following an input to a computer. For example, data to be encoded can include any digital files and folders from a computer. The digital files are encoded and/or converted to a molecular storage code (e.g., nucleotides, amino acids, polymers, atoms, surfaces. The code is written to the physical storage block used to store the data. The stored data is associated with a set of address codes to identify the storage block. In some forms, assembly of the storage blocks is implemented through one or more automated processes, for example, as controlled by a computer. The addresses affixed to the storage block (such that they can be used for subsequent reading, manipulation, selection, and computation, including physical tags, electrostatic or magnetic properties, chemical properties, or optical properties) are recorded in one or more databases or files written to the computer. In some forms, physical placement of the storage blocks with addresses within a pool of other storage blocks for storage and computation can be implemented through one or more automated processes, for example, as controlled by a computer. In some forms, physical separation based on the physical properties, with some storage blocks satisfying the selection criteria and others not, and sorting are implemented through one or more automated processes, for example, as controlled by a computer. Many cycles of this and other selection criteria can be automated or centrally controlled, for example, to take place in parallel or in series. The selection and computation on these tags is recorded in one or more files or databases recorded by the computer. In some forms, physical purification and isolation of selected storage block(s) of interest from the pool is implemented through one or more automated processes, for example, as controlled by a computer. In some forms, the sorted storage block(s) are read out and decoded to digital format by one or more automated or centrally controlled processes, to enable automated retrieval of data from the pool.

A. Devices

In some forms one or more of the apparatus are connected together to facilitate continuous or intermittent flow throughput the apparatus, as a single system. In some forms, the assembly of storage objects from the component parts is implemented with an automated device, or multiple inter-connected devices that combine to produce a system. An exemplary device or system is a microfluidic device or system. In some forms, the mixing of sequence-controlled polymers with one or more feature tags and optionally one or more encapsulating agents is implemented with a microfluidic system.

Microfluidics can be used either in traditional 2-phase droplet form or electro-wetting on dielectric (EWOD) form (Nelson and Kim, Journal of Adhesion Science and Technology, 26 1747-1771 (2012)) to combine, separate, and otherwise manipulate specific pools of the preceding storage objects for either computation or processing or storage/retrieval.

In some forms storage and retrieval or computation of storage objects are carried out using automated systems.

Storage read-out can either be performed using on-chip nanopore-based single-molecule sequencing for DNA/RNA, or PCR-based amplification and sequencing for optical approaches, or other analytical chemical approaches including mass spectrometry, which exploit molecular or nanoparticle charge, size, mass, etc. to read out the information-content or molecular composition of the nanoparticles; affinity or other specific recognition tags as used are also applicable to this workflow. The described methods for the assembly of nucleic acid storage objects can be implemented within a single device. For example, in some forms, the assembly of nucleic acid storage objects is achieved using a device including one or more of

(a) an inlet, for example, to facilitate the in-flow of one or more components of the nucleic acid storage object from an external source;

(b) apparatus for mixing the constituent components, such as a vortex, a shaker, a stir bar, turbulent flow coil, etc.;

(c) apparatus for annealing the constituent components to form an assembled nucleic acid storage object, such as a controllable heat source, a PCR machine, etc.; and

(d) apparatus for purifying the assembled nucleic acid storage object, for example, by affinity chromatography, High Pressure Liquid Chromatography, filtration, etc.

The disclosed compositions and methods can be further understood through the following numbered paragraphs.

1. A sequence-controlled storage object, including

(a) one or more different sequence-controlled polymers, and

(b) a plurality of different feature tags,

wherein the feature tags are present at the surface of the sequence-controlled storage object,

wherein each different feature tag corresponds to a single feature attributable to one or more of the different sequence-controlled polymers,

wherein the single feature to which each different feature tag corresponds is a feature attributable to one or more different of the sequence-controlled polymers,

wherein the plurality of different feature tags collectively corresponds to a plurality of features that are collectively attributable to the plurality of different sequence-controlled polymers,

wherein each of the different feature tags is hybridizably distinguishable from all of the other different feature tags.

2. The sequence-controlled storage object of paragraph 1, wherein each of the plurality of different feature tags is a member of a different set of feature tags, wherein each set of feature tags corresponds to a set of related features.

3. The sequence-controlled storage object of paragraph 2, wherein the members of at least one of the sets of feature tags are similarity-encoded feature tags.

4. The sequence-controlled storage object of paragraph 2, wherein the relative hybridizability of the feature tags in the set is related to the similarity of the features to which the feature tags in the set correspond, wherein feature tags in the set corresponding to more similar features have closer relative hybridizability than feature tags in the set corresponding to less similar features.

5. The sequence-controlled storage object of paragraph 3 or 4, wherein the similarity encoded feature tags of the set of feature tags were similarity encoded by mapping the features to which the feature tags correspond to an n-dimensional hypercube based on the similarity of the features, wherein n is an integer less than or equal to the number of features to which the feature tags correspond, wherein n is a factor of the number of features to which the feature tags correspond.

6. The sequence-controlled storage object of paragraph 5, wherein, prior to mapping the features to which the feature tags correspond, the dimensionality of the features to which the feature tags correspond is reduced, wherein the dimensionality-reduced features are mapped to the hypercube based on the similarity of the dimensionality-reduced features.

7. The sequence-controlled storage object of paragraph 3 or 4, wherein the similarity encoded feature tags of the set of feature tags were similarity encoded by (a) reducing the dimensionality of the features to which the feature tags correspond and (b) mapping the dimensionality-reduced features to an n-dimensional hypercube based on the similarity of the dimensionality-reduced features, wherein n is an integer less than or equal to the number of features to which the feature tags correspond, wherein n is a factor of the number of features to which the feature tags correspond.

8. The sequence-controlled storage object of any one of paragraphs 5-7, wherein the number of edges of the hypercube between the nodes to which any two of the mapped features are mapped is proportional to the similarity of the two features.

9. The sequence-controlled storage object of any one of paragraphs 2-8, wherein the members of at least one of the sets of feature tags are hybridization ordered, wherein the members of the at least one of the sets of feature tags have the same number of nucleotides.

10. The sequence-controlled storage object of any one of paragraphs 2-8, wherein, in at least one of the sets of feature tags, (a) the members of the set of feature tags have the same number of nucleotides and (b) each of the feature tags in the set differs from one or two other feature tags in the set by 1 to x mismatched nucleotides, wherein the mismatched nucleotides are (i) at least two nucleotides from either end of the feature tag and (ii) are separated by at least one matching nucleotide in the feature tags, and wherein x is the number of different nucleotide positions in the feature tags that are varied in the set.

11. The sequence-controlled storage object of paragraph 9 or 10, wherein, independently for one or more sets of the at least one of the sets of feature tags, each feature tag in the set is mismatched from every other feature tag in the set by 1 to w nucleotides, wherein w is an integer from 2 to (y−4)÷2, wherein y is the number of nucleotides in the feature tags in the set, wherein the expression (y−4)÷2 is rounded up.

12. The sequence-controlled storage object of any one of paragraphs 1-11 further including a plurality of different digit tags, wherein the digit tags are present at the surface of the storage object, wherein the digit tags are number encoded.

13. The sequence-controlled storage object of any one of paragraphs 1-11 further including a plurality of different digit tags,

wherein the digit tags are present at the surface of the storage object,

wherein each of the plurality of different digit tags corresponds to the digit value of a different place in a multidigit number, wherein the number of different digit tags included in the storage object equals the number of places in the multidigit number,

wherein each of the plurality of different digit tags is a member of a different set of digit tags, wherein each set of digit tags corresponds to a different place in the multidigit number,

wherein each set of digit tags has a digit tag corresponding to each of the possible digit values of the place in the multidigit number to which the set of digit tags corresponds,

wherein each of the different digit tags is hybridizably distinguishable from all of the other different digit tags in all of the sets of digit tags, wherein each of the different digit tags is hybridizably distinguishable from all of the different feature tags.

14. A sequence-controlled storage object, including

(a) one or more different sequence-controlled polymers, and

(b) a plurality of different digit tags,
wherein the digit tags are present at the surface of the storage object,

wherein each of the plurality of different digit tags corresponds to the digit value of a different place in a multidigit number, wherein the number of different digit tags included in the storage object equals the number of places in the multidigit number,

wherein each of the plurality of different digit tags is a member of a different set of digit tags, wherein each set of digit tags corresponds to a different place in the multidigit number,

wherein each set of digit tags has a digit tag corresponding to each of the possible digit values of the place in the multidigit number to which the set of digit tags corresponds,

wherein each of the different digit tags is hybridizably distinguishable from all of the other different digit tags in all of the sets of digit tags.

15. The sequence-controlled storage object of paragraph 14, wherein the multidigit number corresponds to a feature attributable to one or more of the different sequence-controlled polymers.

16. The sequence-controlled storage object of paragraph 15, wherein the feature attributable to one or more of the different sequence-controlled polymers is a member of a set of related features, wherein each of the members of the set of related features has or can be associated with a different numerical value, wherein the different numerical values corresponds to the level or intensity of a given feature relative to the other features in the set of related features, wherein the multidigit number is equal to, proportional to, or the same as a given number of digits of the numerical value of the feature attributable to one or more of the different sequence-controlled polymers.

17. The sequence-controlled storage object of paragraph 16, wherein the difference in the numerical values with which members of the set of related features have or can be associated are proportional to the similarity of the features in the set of related features.

18. The sequence-controlled storage object of paragraph 15, wherein the multidigit number is arbitrarily assigned to the feature attributable to one or more of the different sequence-controlled polymers to which the multidigit number corresponds.

19. The sequence-controlled storage object of any one of paragraphs 15-18, wherein the multidigit number is the same as a given number of digits of the numerical value of the feature attributable to one or more of the different sequence-controlled polymers starting from the most significant digit of the numerical value.

20. The sequence-controlled storage object of any one of paragraphs 14-19, wherein each set of digit tags has the same number of members as the mathematical base in which the multidigit number is expressed.

21. The sequence-controlled storage object of any one of paragraphs 1-20 further including one or more encapsulating agents,

wherein the encapsulating agent coats or encapsulates the sequence-controlled polymers, wherein the encapsulating reagent can be reversibly removed through chemical or mechanical treatment.

22. The sequence-controlled storage object of paragraph 21, wherein the feature tags are included in one or more of the encapsulating agents.

23. The sequence-controlled storage object of paragraph 21 or 22, wherein the one or more encapsulating agents are selected from the group including natural polymers and synthetic polymers, or combinations thereof.

24. The sequence-controlled storage object of any one of paragraphs 21-23, wherein one or more encapsulating agents are selected from the group including proteins, polysaccharides, lipids, nucleic acids, inorganic coordination polymers, metal-organic frameworks, covalent organic frameworks, inorganic coordination cages, covalent organic coordination cages, elastomers, thermoplasts, synthetic fibers, or any derivatives thereof.

25. The sequence-controlled storage object of any one of paragraphs 1-24, wherein at least one of the sequence-controlled polymers is a single stranded nucleic acid, and

wherein the nucleic acid is folded into a three-dimensional polyhedral nanostructure including two nucleic acid helices that are joined by either anti-parallel or parallel crossovers spanning each edge of the structure,

wherein the three-dimensional polyhedral structure is formed from single stranded nucleic acid staple sequences hybridized to the single stranded nucleic acid including bit-stream data,

wherein the single stranded nucleic acid including bit-stream data is routed through the Eulerian cycle of the network defined by the vertices and lines of the polyhedral structure,

wherein the nanostructure includes at least one edge including a double stranded or single-stranded crossover,

wherein the location of the double strand crossover is determined by the spanning tree of the polyhedral structure,

wherein the staple sequences are hybridized to the vertices, edges and double strand crossovers of the single stranded nucleic acid including bit-stream data to define the shape of the nanostructure, and

wherein one or more of the staple sequences includes one or more feature tag sequences.

26. The sequence-controlled storage object of paragraph 25, wherein a staple strand includes from 14 to 1,000 nucleotides, inclusive.

27. The sequence-controlled storage object of paragraph 25, wherein the single-stranded nucleic acid includes approximately 100 to 1,000,000 nucleotides, inclusive.

28. The sequence-controlled storage object of any one of paragraphs 25-27, wherein one or more staple strands include one or more feature tag sequences at the 5′ end, at the 3′ end, or at both the 5′ end and at the 3′ end.

29. The sequence-controlled storage object of paragraph 28, wherein the one or more feature tag sequences include one or more overhang oligonucleotide sequences.

30. The sequence-controlled storage object of paragraph 28 or 29, wherein the one or more feature tag sequences include oligonucleotide sequences complementary to one or more feature tag sequences attached to a different sequence-controlled storage object.

31. The sequence-controlled storage object of any one of paragraphs 28-30, further including one or more additional sequence-controlled storage objects bound thereto.

32. A method of storing desired sequence-controlled polymers as a sequence-controlled storage object, including

(a) assembling a sequence-controlled storage object from
(i) one or more different sequence-controlled polymers, and
(ii) a plurality of different feature tags, and
(iii) optionally one or more encapsulating agents,
wherein the feature tags are present at the surface of the sequence-controlled storage object,
wherein each different feature tag corresponds to a single feature attributable to one or more of the different sequence-controlled polymers,
wherein the single feature to which each different feature tag corresponds is a feature attributable to one or more different of the sequence-controlled polymers,
wherein the plurality of different feature tags collectively corresponds to a plurality of features that are collectively attributable to the plurality of different sequence-controlled polymers,
wherein each of the different feature tags is hybridizably distinguishable from all of the other different feature tags; and

(b) storing the sequence-controlled storage object.

33. The method of paragraph 32 further including the step of

(c) retrieving the desired sequence-controlled polymers.

34. The method of paragraph 33, wherein retrieving the desired sequence-controlled polymers in step (c) includes isolating one or more sequence-controlled storage objects from a pool of sequence-controlled storage objects.

35. The method of paragraph 34, wherein selection is determined by the sequence of one or more feature tags on the sequence-controlled storage object, the shape of the sequence-controlled storage object, affinity to a functionalized group bound to the sequence-controlled storage object, or combinations thereof.

36. The method of paragraph 35, further including the step of modifying the isolated sequence-controlled storage object by addition of one or more different feature tags.

37. The method of paragraph 36, wherein addition of one or more different feature tags includes refolding, or re-organizing the sequence-controlled storage object with one or oligonucleotides including the different feature tags.

38. The method of paragraph 37, wherein one or more sequence-controlled storage objects are isolated from a pool of sequence-controlled storage objects using Boolean logic.

39. The method of paragraph 38, wherein Boolean NOT logic is used to delete one or more sequence-controlled storage objects from an object pool.

40. The method of any one of paragraphs 32-39, further including the step of

(f) accessing the desired sequence-controlled polymers.

41. The method of any one of paragraphs 32-40, wherein storing the sequence-controlled storage object in step (b) further includes one or more of dehydrating, lyophilizing, or freezing the sequence-controlled storage object.

42. The method of paragraph 41, wherein storing the sequence-controlled storage object in step (b) further includes one or more of rehydrating or thawing the sequence-controlled storage object for processing.

43. The method of any one of paragraphs 32-42, wherein storing the sequence-controlled storage objects includes storage in a matrix selected from the group including cellulose, paper, microfluidics, bulk 3D solution, on surfaces using electrical forces, on surfaces using magnetic forces, encapsulated in inorganic or organic salts, and combinations thereof.

44. The method of any one of paragraphs 32-43, wherein storing the sequence-controlled storage object in step (b) further includes digitally processing droplets containing sequence-controlled storage objects.

45. A method of automating the assembly of the sequence-controlled storage object of any one of paragraphs 1-31 including using a device with flow, the device including

(a) means for flowing in the constituent components of the sequence-controlled storage object,

(b) means for mixing the constituent components,

wherein the means for mixing is operatively connected to the means for flowing,

(c) means for annealing the constituent components to form an assembled sequence-controlled storage object,

wherein the means for annealing is operatively connected to the means for mixing, and

(d) means for purifying the assembled sequence-controlled storage object,

wherein the means for purifying is operatively connected to the means for annealing.

46. The method of paragraph 45 further including

(e) means for introducing encapsulating agents to store the sequence-controlled object,

(f) means for introducing a plurality of feature tags attributable to the sequence-controlled polymer,

(g) means for selecting encapsulated sequence-controlled objects from an object pool, wherein the means of selection can be performed using Boolean logic, and

(h) means for removing the encapsulating agent to retrieve the sequence-controlled storage object.

The present invention will be further understood by reference to the following non-limiting examples.

### EXAMPLES

### Example 1

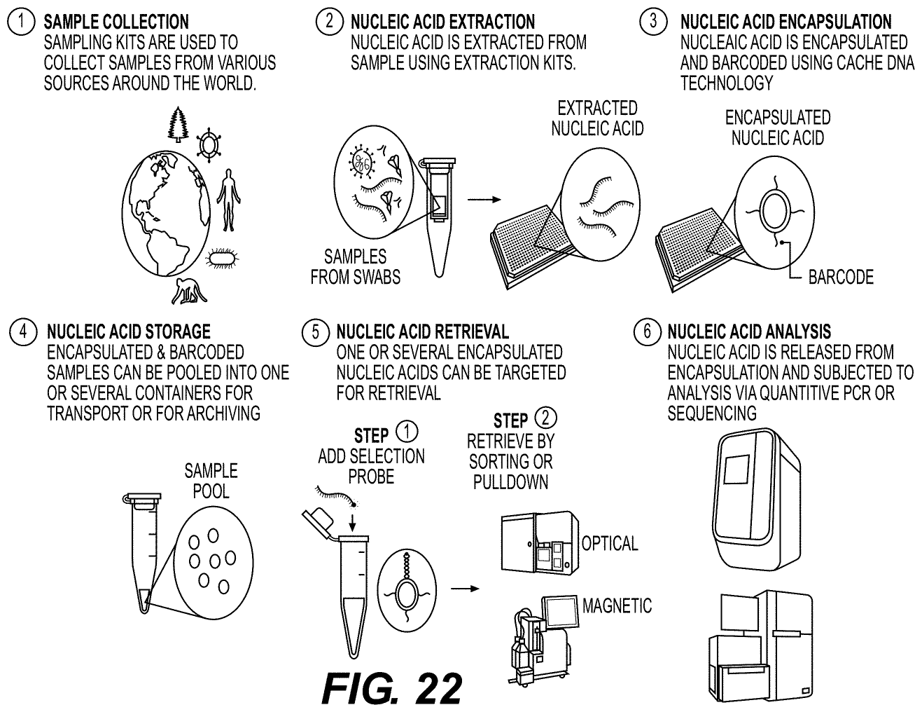

An overview of the process for sample collection, nucleic acid extraction, nucleic acid encapsulation, nucleic acid storage and retrieval is set forth in FIG. 22.

In one example, a volume of 10 μL of Bos taurus nucleic acid with a concentration of 100 ng μL⁻¹ is added to an LoBind Eppendorf tube containing 900 μL of nuclease-free water, as depicted in FIG. 23A. A volume of 10 μL of 50 mg mL⁻¹ trimethylammonium-modified silica particles are then added and mixed gently by flipping the tube several times. Trimethyl-3-trimethoxysilyl chloride and tetraethoxysilane are then added and mixed on a thermal mixer for 4 days at room temperature. Upon completion of the encapsulation, the mixture is centrifuged, and the pellet is washed with ethanol five times. The pellet is resuspended in 900 μL ethanol with vortexing and 50 μL of 3-(2-aminoethylamino)propyldimethoxymethylsilane was added. The mixture was mixed for 24 hours on a thermal mixer at room temperature. After surface modification of the encapsulated nucleic acid, the mixture was centrifuged, and the pellet was washed with dimethylformamide five times. The pellet is resuspended in 900 μL dimethylformamide with vortexing, and 50 μL of 10 mg mL⁻¹ 2-azidoacetic acid N-hydroxysuccinimide (NHS) ester was added. The mixture was again mixed for 24 hours on a thermal mixer at room temperature. After azide conversion, the mixture was centrifuged and the pellet was washed with dimethylformamide five times. The pellet is resuspended in 900 μL dimethylformamide with vortexing and 10 μL of 100 mg mL⁻¹ dibenzocyclooctyne-PEG13-NHS hydroxysuccinimide ester was added. The mixture was mixed for 4 hours on a thermal mixer at room temperature. After PEG modification, the mixture was centrifuged and the pellet was washed with dimethylformamide five times. The pellet is resuspended in 200 μL dimethylformamide with vortexing. A volume of 800 μL containing 0.050 M phosphate buffer and 6 μM of each amine-modified barcodes assigned to the labels “Eukaryote” (AACGATTGTTATGCCCCTAACTCAG) (SEQ ID NO:4), “Animalia” (ATGGACGACTTGGGACGGGTATCAA) (SEQ ID NO:5), “Bos taurus” (TAATGTGGCTTGGCTCACCGCTAGG) (SEQ ID NO:6), “2021-01-05” (CGATGTAGTCATCCCGATGTGCTGG) (SEQ ID NO:7). The mixture was again mixed for 24 hours on a thermal mixer at room temperature. After molecular barcode labeling, the mixture was centrifuged, and the pellet was washed five times with 1×PBS with 0.1% Tween-20.

The exact encapsulation and barcoding procedure are repeated for additional samples and all encapsulated samples are subsequently pooled to form the molecular library (see, for example, FIGS. 23A-23B). Querying the molecular database proceeds by the addition of probes that contain chemical or biochemical markers that are used for downstream sorting. The addition of hydrofluoric acid releases samples. Desalting using spin-columns removes excess salt, and the sample is now ready for subsequent sequencing or amplification reactions.

### Example 2

In another example, samples are encapsulated instead in synthetic or biological polymers using emulsions. Samples in the aqueous phase, which may contain water-soluble monomers or polymers for crosslinking, are made into droplets in surfactant-containing oil using microfluidic or millifluidic approaches (FIGS. 25A-24C). The polymerization and crosslinking reactions are allowed to occur until all monomers are used up. The emulsions are broken up post-polymerization and the barcodes are affixed chemically/biochemically on the surface of the capsule through the polymer's non-terminated ends.

As an example, one million copies of the SARS-CoV-2 RNA genome dissolved in nuclease-free water that contains 2 mM Ca2+ and 2% (w/w) low-viscosity alginate is flowed into a channel that is connected to T-junction where surfactant-containing oil is being flowed. Methylene blue was added into the aqueous phase to visualize the formation of the droplet in real-time (FIG. 25C).

### Example 3

In another example, sample encapsulation and barcoding are performed in a single-step using multi-stage microfluidics (FIGS. 25A-25B). The aqueous phase containing the nucleic acids flows into oil containing a surfactant and water-insoluble monomers, crosslinkers, and polymerization initiators. The droplet passes through another aqueous fluid stream containing the barcodes labeled with chemical/biochemical handles for attachment to the polymer's non-terminated ends. The reactions are allowed to proceed until encapsulation polymerization is finished.

### Example 4

In another sample, encapsulated samples can be selected from the solution using isothermal chemical/biochemical amplification. Probe strands that contain trigger sequences or modified with biochemical catalysts or co-factors are hybridized on samples that include the desired barcode. Molecular labels, including but not limited to dyes and chemical/biochemical affinity tags, are amplified and improve the proposed system's sorting efficiency.

### Example 5: Design of Nucleic Acid Storage Object Superstructures

### Methods

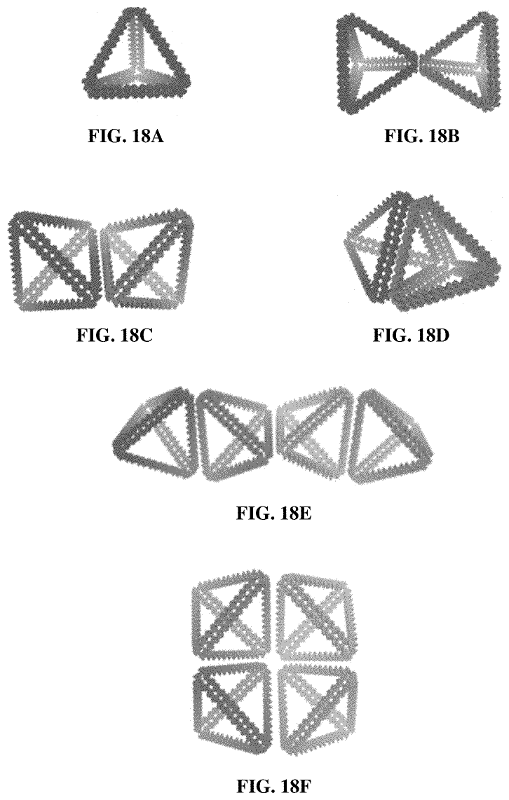

Super-structuring by complementary overhangs was tested using two tetrahedra. 3′ single-strand DNA overhangs off two different staple nicks on the same edge of a tetrahedron with edge-length 63 nucleotides were generated, with a scaffold of a sequence amplified from M13 phage genomic DNA. Sequences complementary to the two overhangs on the first tetrahedron (tet-A) were generated and placed as 3′ single-strand DNA overhangs of two different nicks on the same edge of a second tetrahedron, with a scaffold also amplified from M13 genomic DNA (tet-B). These two structures with complementary overhangs were separately folded and purified, and then pooled and slowly annealed over two hours from 43° C. to 25° C. Verification of superstructuring was done via gel shift mobility assay on 2% agarose and visualized under UV light with SYBR Safe DNA stain. The gel showed a shift indicative of quantitative dimer formation. This same exact procedure is used for superstructuring NSOs by use of complementary strands per edge. Further, a series of 4 tetrahedra were structured such that two overhangs per edge were made complementary to a second tetrahedron, which had opposite to that edge a second set of two overhangs complementary to a second dimer-set. Thus 2 tetrahedra dimers were annealed to each other to form a tetramer of tetrahedra (depicted in FIGS. 18B-18D). The same scaffold sequence was used to form a set of tetrahedral of the same scaffold but with different addresses that had curvature to the superstructure that caused the 4 tetrahedra to close back to itself. Thus NSOs can be assembled to be in elongated or closed superstructures dependent on the exposed addresses.

### Results

To demonstrate NSO superstructuring, NSOs were brought together at their vertices, along their edges, or at their faces using overhang addressing. Exemplary tetrahedra were demonstrated as coming together in larger superstructures by a Gel mobility shift assays indicating superstructuring as compared to monomer NSOs, dimer NSOs, and tetramer NSOs, respectively. Extended tetramers were addressed to come together along the edges via complementarity, as determined by transmission electron microscopy showing the extended configuration. The same tetrahedra, but with different addresses, were observed as forming different compact configurations.

### Example 6: Paper Storage of Nucleic Acid Storage Object Structures

### Methods

Storage of NSOs on paper as a medium for long-term preservation was tested. Whatman paper type 42 was cut to mm scale (typically 2 mm×5 mm) and saturated with 15 μL 1×TAE+12 mM MgCl₂+1% PEG 8000 w/v. The paper was then dried under vacuum in the presence of desiccant. 15 μL of 40 nM DNA nanostructures (tetrahedra with edge-length 63 nucleotides) was then added to the paper and dried under vacuum. After at least 14 hours at room temperature the paper was transferred to a separate tube and washed with 15 μL folding buffer, and the solution was separated from the paper by centrifugation. Gel mobility shift assays indicated structural stability. Likewise, NSOs can be stored for long lengths of time and resuspended as needed.

### Results

NSOs were dried and stored to paper that was pretreated with 1% Polyethylene glycol 8000 before exposed to NSOs. The NSOs transferred to the paper were later rehydrated, and were still present in assembled form, as indicated by a Gel-shift assay. Exemplary paper tabs containing dried NSOs were stored within a single Eppendorf tube.

### Example 7: Metal Oxide Storage of Nucleic Add Storage Object Structures

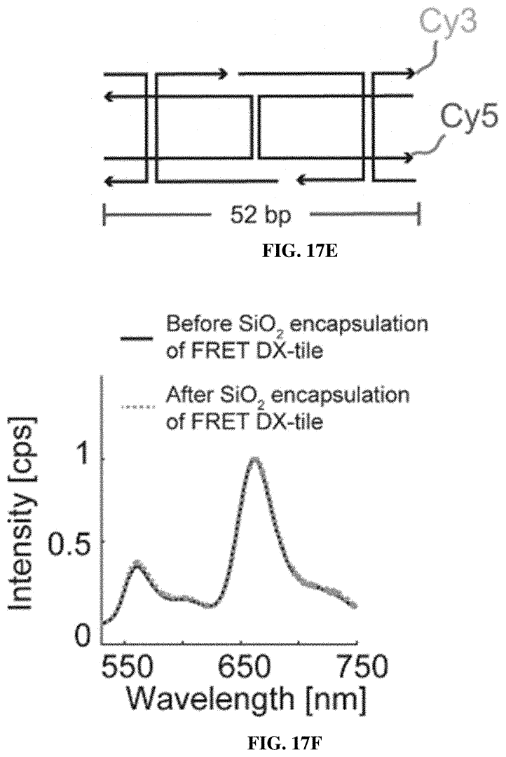

Experiments to demonstrate the packaging and accessibility of nucleic acids by encapsulation or coating in a non-nucleic acid polymer were carried out. Briefly, nucleic acids were encased within a polymer, addressed with one or more tags (depicted in FIGS. 4A-4D and FIGS. 17A-17D).

### Methods and Materials

Preparation of Silica Particles

Silica particles were prepared by mixing 800 μL of 25% w/w ammonium hydroxide, 800 μL of tetraethoxysilane, and 500 μL of distilled water in 18 mL of water. The mixture was shaken on a platform orbital shaker at 500 rpm for 6 hours at room temperature. The mixture was then centrifuged at 9,000 g for 20 minutes at room temperature and the supernatant was discarded. The silica pellets were re-dispersed in solution by adding a total of 20 mL of isopropanol then sonicating for 1 minute at room temperature and vortexing for 5 seconds to get a homogenous colloidal solution. The mixture was again centrifuged at 9,000 g for 20 minutes at room temperature and the supernatant was again discarded. The pellet was re-dispersed in solution by adding a total of 4 mL of isopropanol, sonicating for 1 minute, and vortexing for 5 seconds until a homogenous dispersion is again achieved.

Modification of Silica Particles to Facilitate Adsorption of DNA Particles

The silica particles were immediately modified by taking a 1 mL aliquot of the silica particles and adding 10 μL of 50% w/w N-trimethoxylsilylpropyl-N,N,N-trimethylammonium (TMAPS) chloride in methanol. The mixture was shaken on a platform orbital shaker at 500 rpm for 12 hours at room temperature. The mixture was then centrifuged at 21,500 g for 4 minutes discarding the supernatant. The modified silica pellets were suspended with 1 mL of isopropanol, sonicated for 1 minute, and vortexed for 5 seconds to achieve a homogenous solution. The mixture was again centrifuged at 21,500 g for 4 minutes and the supernatant was again discarded. The same washing procedure was repeated twice to remove residual TMAPS in solution.

Encapsulation of DNA Particles

A double-crossover (DX) tile modified with Cy3 and Cy5 energy transfer pair as a readout was encapsulated by adding 320 μL of 50 μg mL⁻¹ Cy3 and Cy5-modified DX tile to 700 μL of water and 35 μL of functionalized silica particles (FIG. 17D). The mixture was shaken on a microtube revolver for 3 minutes at room temperature then centrifuged at 21,500 g for 4 minutes discarding the supernatant. The silica pellets were then suspended with 1 mL of DNAse-free water, sonicated for 1 minute at room temperature, and vortexed for 5 seconds. The mixture was then centrifuged at 21,500 g for 4 minutes discarding the supernatant. The silica pellets were re-suspended with 500 μL of DNAse-free water, sonicated for 1 minute at room temperature, and vortexed for 5 seconds. To this mixture, a volume of 0.5 μL TMAPS was added and mixed by vortexing for 5 seconds. An additional 0.5 μL of TEOS was then added. The mixture was shaken on a microtube revolver for 4 hours at room temperature then 4 μL of TEOS was added. The mixture was further shaken on a microtube revolver for 4 days. The mixture was centrifuged at 21,500 g for 4 minutes discarding the supernatant. The silica-encapsulated DX tile pellet was re-suspended with 500 μL of DNAse-free water, sonicated for 1 minute at room temperature, and vortexed for 5 seconds. The mixture was again centrifuged at 21,500 g for 4 minutes discarding the supernatant. The pellet was re-suspended with 100 μL of DNAse-free water, sonicated for 1 minute at room temperature, and vortexed for 5 seconds. The DX-tile is finally encapsulated. Schematic illustrations of the silica encapsulation of nucleic acid storage blocks are depicted in FIGS. 17A-17D.

The encapsulated particles were drop casted on paper to test the protective particles of silica with DNA. A volume of 10 μL was dropped on paper and was allowed to dry in ambient temperature. A volume of 10 μL of DNA denaturants (0.1 M HCl, 0.1 M NaOH, and DNAse) was then added and allowed to dry again at room temperature.

### Results

The surface of the silica particles was modified to allow adsorption of DNA storage objects, such that the modified silica particles act as a scaffold for the nucleic acid storage blocks to bind onto.

The nucleic acid storage blocks are first adsorbed to the surface-modified silica particles, then a secondary silica shell is appended onto the silica with the nucleic acid storage blocks adsorbed. A schematic of an exemplar DNA assembly (a double-crossover or DX tile) containing Cy3 and Cy5 energy transfer pair as a readout for monitoring the structure of the DX tile is provided in FIG. 17E. This shell provides environmental protection for the nucleic acid storage blocks.

Assessment of the encapsulated particles was carried out by comparing silica-encapsulated particles with non-encapsulated nanoparticles under UV illumination filtering only Cy5 fluorescence using a longpass filter. No change in the emission spectra of the DX tile upon completion of the encapsulation step showing that the encapsulation process does not perturb the structure of the DX tile (see FIG. 17F).

To assess protection of DNA storage objects by the silica encapsulation process, silica-encapsulated DX tiles were absorbed onto a strip of paper and exposed to 0.1 M NaOH, 0.1 M HCl, and DNAse. The silica-coated paper was excited at 400 nm and the emission was selected using a 650 nm longpass filter.

### Example 8: Microfluidic Device for Automated Assembly of Nucleic Add Storage Object Structures

### Methods and Materials

A system for the automated assembly of nucleic acid storage objects was designed and assembled to include the device 3D printed to a size of 10 cm by 4 cm, with 3 input ports, a mixer and annealer over a copper plate, and 3 output ports, with one foot of the copper plate in 80° C. water bath and the other foot of the copper plate in ice water.

The input port was connected to a fluid pump and the output was connected to a fraction collector tube, with the fluid flow passing first from the reagents, including scaffold nucleic acid, tagged staple strands and staples, into the mixer, then from into and through the annealer into a fraction collector. Within the annealer the fluid passes from high temperature to a low temperature. Fractions were collected and purified by filtration.

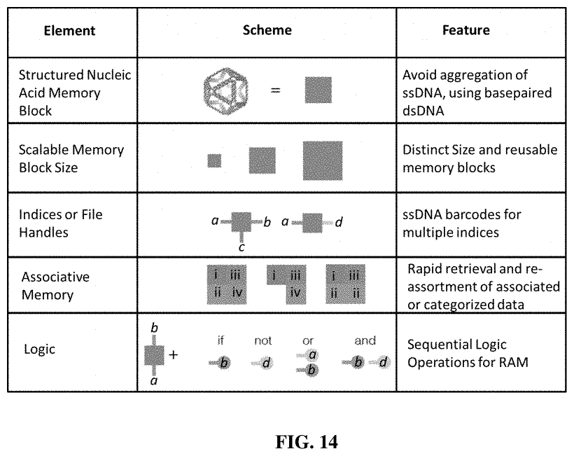

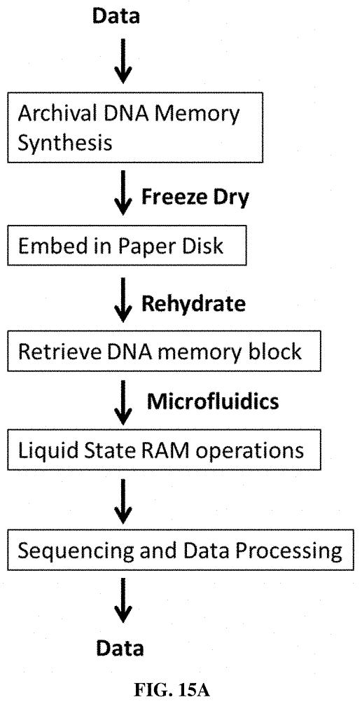

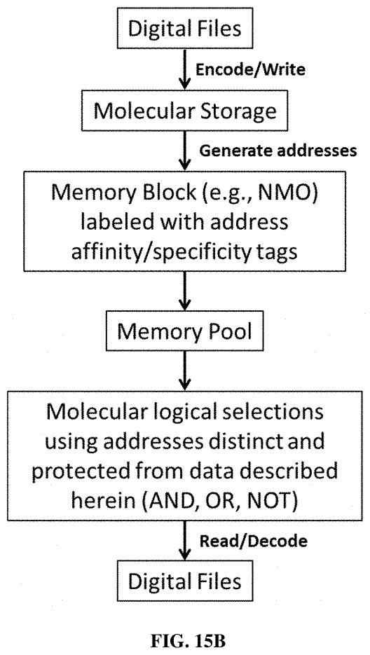

The DNA nanoparticles annealing reaction in the auto-assembler was realized in 1.2 mL reaction volume with ssDNA scaffold at a concentration of 80 nM and a 15× excess of staple strands in Tris-Acetate EDTA-MgCl₂ buffer (40 mM Tris, 20 mM acetic acid, 2 mM EDTA, 12 mM MgCl₂, pH 8.0). Before injection of the sample the device was washed with 4 mL of folding buffer at a flow rate of 100 μL/min. For the sample injection, the flow rate was maintained at 10 μL/min through the auto-assembler channel using a Gilson, Inc. MINIPULS® 3 peristaltic pump. The temperature gradient in the auto-assembler was created by connecting one of the extremity of the copper plate (Denaturation area) to an 80° C. water bath and the collecting extremity of the copper plate to a cold water bath kept at 4° C. Sample collection was regularly monitored using a nanodrop. A schematic representation of the automated system is depicted in FIG. 12. The exemplary work-flow for implementation of automated systems within exemplary microfluidic devices are also depicted in FIGS. 13, 14 and 15.

Output from the auto-assembler was tested by gel on a 1% agarose gel supplemented with 12 mM MgCl₂.

### Results

The resulting nanostructure assemblies were assessed by gel electrophoresis. The folding of assembled objects was determined by visual observation of gel bands in each lane of the gel corresponding to scaffold nucleic acid alone, scaffold mixed at room temperature with staples, scaffold and staples mixed and annealed over 3 hours in a thermal cycler, and scaffold and staples mixed and annealed over 3 hours on the auto-assembler.

Gel-shift assays were used to test folding. Lanes corresponding to the scaffold and staples mixed and annealed over 3 hours in a thermal cycler were of equal position and intensity to those in the gel lane corresponding to the scaffold and staples mixed and annealed over 3 hours on the auto-assembler. The experiment demonstrated the efficacy of the auto-assembly system is at least as efficient as assembly using a thermal cycler.

We claim:

1. A sequence-controlled storage object, comprising
(a) one or more different sequence-controlled polymers; and
(b) a plurality of different feature tags,
wherein the feature tags are present at the surface of the sequence-controlled storage object,
wherein each different feature tag corresponds to a single feature attributable to one or more of the different sequence-controlled polymers,
wherein the single feature to which each different feature tag corresponds is a feature attributable to one or more of the different sequence-controlled polymers,
wherein the plurality of different feature tags collectively corresponds to a plurality of features that are collectively attributable the one or more different sequence-controlled polymers, and
wherein each of the different feature tags is hybridizably distinguishable from all of the other different feature tags.

2. The sequence-controlled storage object of claim 1, wherein each of the plurality of different feature tags is a member of a different set of feature tags, wherein each set of feature tags corresponds to a set of related features,
optionally wherein
(i) the members of at least one of the sets of feature tags are similarity-encoded feature tags; and/or
(ii) the members of at least one of the sets of feature tags are hybridization ordered, and the members of the at least one of the sets of feature tags have the same number of nucleotides.

3. The sequence-controlled storage object of claim 2, wherein the relative hybridizability of the feature tags in the set is related to the similarity of the features to which the feature tags in the set correspond, and
wherein feature tags in the set corresponding to more similar features have closer relative hybridizability than feature tags in the set corresponding to less similar features.

4. The sequence-controlled storage object of claim 3, wherein the similarity encoded feature tags of the set of feature tags are similarity encoded by mapping the features to which the feature tags correspond to an n-dimensional hypercube based on the similarity of the features,
wherein n is an integer less than or equal to the number of features to which the feature tags correspond, and
wherein n is a factor of the number of features to which the feature tags correspond,
optionally wherein, prior to mapping the features to which the feature tags correspond, the dimensionality of the features to which the feature tags correspond is reduced, and
wherein the dimensionality-reduced features are mapped to the hypercube based on the similarity of the dimensionality-reduced features.

5. The sequence-controlled storage object of claim 3, wherein the similarity encoded feature tags of the set of feature tags are similarity encoded by
(a) reducing the dimensionality of the features to which the feature tags correspond; and
(b) mapping the dimensionality-reduced features to an n-dimensional hypercube based on the similarity of the dimensionality-reduced features,
wherein n is an integer less than or equal to the number of features to which the feature tags correspond,
wherein n is a factor of the number of features to which the feature tags correspond,
optionally wherein the number of edges of the hypercube between the nodes to which any two of the mapped features are mapped is proportional to the similarity of the two features.

6. The sequence-controlled storage object of claim 2, wherein, in at least one of the sets of feature tags,
(a) the members of the set of feature tags have the same number of nucleotides; and
(b) each of the feature tags in the set differs from one or two other feature tags in the set by 1 to x mismatched nucleotides, wherein the mismatched nucleotides are
(i) at least two nucleotides from either end of the feature tag; and
(ii) are separated by at least one matching nucleotide in the feature tags, and wherein x is the number of different nucleotide positions in the feature tags that are varied in the set.

7. The sequence-controlled storage object of claim 2, wherein, independently for one or more sets of the at least one of the sets of feature tags, each feature tag in the set is mismatched from every other feature tag in the set by 1 to w nucleotides,
wherein w is an integer from 2 to (y−4)÷2,
wherein y is the number of nucleotides in the feature tags in the set, and
wherein the expression (y−4)÷2 is rounded up.

8. The sequence-controlled storage object of any one of claim 1, further comprising a plurality of different digit tags, wherein the digit tags are present at the surface of the storage object, and wherein the digit tags are number encoded,
optionally wherein each of the plurality of different digit tags corresponds to the digit value of a different place in a multidigit number,
wherein the number of different digit tags comprised in the storage object equals the number of places in the multidigit number,
wherein each of the plurality of different digit tags is a member of a different set of digit tags,
wherein each set of digit tags corresponds to a different place in the multidigit number,
wherein each set of digit tags has a digit tag corresponding to each of the possible digit values of the place in the multidigit number to which the set of digit tags corresponds,
wherein each of the different digit tags is hybridizably distinguishable from all of the other different digit tags in all of the sets of digit tags,
wherein each of the different digit tags is hybridizably distinguishable from all of the different feature tags.

9. A sequence-controlled storage object, comprising
(a) one or more different sequence-controlled polymers; and
(b) a plurality of different digit tags,
wherein the digit tags are present at the surface of the storage object,
wherein each of the plurality of different digit tags corresponds to the digit value of a different place in a multidigit number,
wherein the number of different digit tags comprised in the storage object equals the number of places in the multidigit number,
wherein each of the plurality of different digit tags is a member of a different set of digit tags, wherein each set of digit tags corresponds to a different place in the multidigit number,
wherein each set of digit tags has a digit tag corresponding to each of the possible digit values of the place in the multidigit number to which the set of digit tags corresponds,
wherein each of the different digit tags is hybridizably distinguishable from all of the other different digit tags in all of the sets of digit tags,
optionally wherein each set of digit tags has the same number of members as the mathematical base in which the multidigit number is expressed.

10. The sequence-controlled storage object of claim 9, wherein the multidigit number corresponds to a feature attributable to one or more of the different sequence-controlled polymers.

11. The sequence-controlled storage object of claim 10, wherein the feature attributable to one or more of the different sequence-controlled polymers is a member of a set of related features,
wherein each of the members of the set of related features has or can be associated with a different numerical value,
wherein the different numerical values correspond to the level or intensity of a given feature relative to the other features in the set of related features,
wherein the multidigit number is equal to, proportional to, or the same as a given number of digits of the numerical value of the feature attributable to one or more of the different sequence-controlled polymers,
optionally wherein
(i) the difference in the numerical values with which members of the set of related features have or can be associated are proportional to the similarity of the features in the set of related features;
(ii) the multidigit number is arbitrarily assigned to the feature attributable to one or more of the different sequence-controlled polymers to which the multidigit number corresponds; or
(iii) the multidigit number is the same as a given number of digits of the numerical value of the feature attributable to one or more of the different sequence-controlled polymers starting from the most significant digit of the numerical value.

12. The sequence-controlled storage object of any one of claim 1, further comprising one or more encapsulating agents,
wherein the encapsulating agent coats or encapsulates the sequence-controlled polymers, wherein the encapsulating reagent can be reversibly removed through chemical or mechanical treatment, optionally wherein
(i) the feature tags are comprised in one or more of the encapsulating agents; and/or
(ii) the one or more encapsulating agents are selected from the group consisting of natural polymers and synthetic polymers, or combinations thereof; and/or
(iii) one or more encapsulating agents are selected from the group consisting of proteins, polysaccharides, lipids, nucleic acids, inorganic coordination polymers, metal-organic frameworks, covalent organic frameworks, inorganic coordination cages, covalent organic coordination cages, elastomers, thermoplasts, synthetic fibers, or any derivatives thereof.

13. The sequence-controlled storage object of any one of claim 1, wherein at least one of the sequence-controlled polymers is a single stranded nucleic acid, and
wherein the nucleic acid is folded into a three-dimensional polyhedral nanostructure comprising two nucleic acid helices that are joined by either anti-parallel or parallel crossovers spanning each edge of the structure,
wherein the three-dimensional polyhedral structure is formed from single stranded nucleic acid staple sequences hybridized to the single stranded nucleic acid including bit-stream data,
wherein the single stranded nucleic acid including bit-stream data is routed through the Eulerian cycle of the network defined by the vertices and lines of the polyhedral structure,
wherein the nanostructure comprises at least one edge including a double stranded or single-stranded crossover,
wherein the location of the double strand crossover is determined by the spanning tree of the polyhedral structure,
wherein the staple sequences are hybridized to the vertices, edges and double strand crossovers of the single stranded nucleic acid including bit-stream data to define the shape of the nanostructure, and
wherein one or more of the staple sequences comprises one or more feature tag sequences.

14. The sequence-controlled storage object of claim 13, wherein a staple strand comprises from 14 to 1,000 nucleotides, inclusive, or
wherein the single-stranded nucleic acid comprises approximately 100 to 1,000,000 nucleotides, inclusive, or combinations thereof.

15. The sequence-controlled storage object of claim 13, wherein one or more staple strands include one or more feature tag sequences at the 5′ end, at the 3′ end, or at both the 5′ end and at the 3′ end.

16. The sequence-controlled storage object of claim 15, wherein the one or more feature tag sequences comprise one or more overhang oligonucleotide sequences,
optionally wherein the one or more feature tag sequences comprise oligonucleotide sequences complementary to one or more feature tag sequences attached to a different sequence-controlled storage object.

17. The sequence-controlled storage object of claim 13, further comprising one or more additional sequence-controlled storage objects bound thereto.

18. A method of storing desired sequence-controlled polymers as a sequence-controlled storage object, comprising
(a) assembling a sequence-controlled storage object from
(i) one or more different sequence-controlled polymers, and
(ii) a plurality of different feature tags, and
(iii) optionally one or more encapsulating agents,
wherein the feature tags are present at the surface of the sequence-controlled storage object,
wherein each different feature tag corresponds to a single feature attributable to one or more of the different sequence-controlled polymers,
wherein the single feature to which each different feature tag corresponds is a feature attributable to one or more different of the sequence-controlled polymers,
wherein the plurality of different feature tags collectively corresponds to a plurality of features that are collectively attributable to the plurality of different sequence-controlled polymers,
wherein each of the different feature tags is hybridizably distinguishable from all of the other different feature tags; and
(b) storing the sequence-controlled storage object,
optionally further comprising the step of
(c) retrieving the desired sequence-controlled polymers.

19. The method of claim 18, wherein retrieving the desired sequence-controlled polymers in step (c) comprises selecting one or more sequence-controlled storage objects from a pool of sequence-controlled storage objects,
wherein the selecting comprises isolating the storage object based on the sequence of one or more feature tags on the sequence-controlled storage object, the shape of the sequence-controlled storage object, affinity to a functionalized group bound to the sequence-controlled storage object, or combinations thereof.

20. The method of claim 18, further comprising the step of
(d) modifying the isolated sequence-controlled storage object by addition of
one or more different feature tags,
optionally wherein addition of one or more different feature tags includes refolding, or re-organizing the sequence-controlled storage object with one or more oligonucleotides including the different feature tags.

21. The method of claim 19, wherein one or more sequence-controlled storage objects are isolated from a pool of sequence-controlled storage objects using Boolean logic,
optionally wherein Boolean NOT logic is used to delete one or more sequence-controlled storage objects from an object pool.

22. The method of claim 20, further comprising the step of
(f) accessing the desired sequence-controlled polymers.

23. The method any one of claim 18, wherein storing the sequence-controlled storage object in step (b) further comprises one or more of dehydrating, lyophilizing, or freezing the sequence-controlled storage object,
optionally further comprising one or more of rehydrating or thawing the sequence-controlled storage object for processing.

24. The method of claim 23, wherein storing the sequence-controlled storage objects comprises storage in a matrix selected from the group consisting of cellulose, paper, microfluidics, bulk 3D solution, on surfaces using electrical forces, on surfaces using magnetic forces, encapsulated in inorganic or organic salts, and combinations thereof.

25. The method of claim 18, wherein storing the sequence-controlled storage object in step (b) further comprises digitally processing droplets containing sequence-controlled storage objects.

26. A method of automating the assembly of the sequence-controlled storage object of claim 1 comprising using a device with flow, the device comprising
(a) means for flowing in the constituent components of the sequence-controlled storage object;
(b) means for mixing the constituent components,
wherein the means for mixing is operatively connected to the means for flowing;
(c) means for annealing the constituent components to form an assembled sequence-controlled storage object,
wherein the means for annealing is operatively connected to the means for mixing; and
(d) means for purifying the assembled sequence-controlled storage object,
wherein the means for purifying is operatively connected to the means for annealing;
optionally further comprising
(e) means for introducing encapsulating agents to store the sequence-controlled object;
(f) means for introducing a plurality of feature tags attributable to the sequence-controlled polymer;
(g) means for selecting encapsulated sequence-controlled objects from an object pool,
wherein the means of selection can be performed using Boolean logic; and
(h) means for removing the encapsulating agent to retrieve the sequence-controlled storage object.


SEQUENCE LISTING (7 sequences)

SEQ ID NO: 1 - DNA, 15 bp, Artificial Sequence
  Synthetic nucleic acid
cccatcgtgtcatta

SEQ ID NO: 2 - DNA, 25 bp, Artificial Sequence
  Synthetic nucleic acid sequcne
gccttgtatgtgaatatccgtgtca

SEQ ID NO: 3 - DNA, 25 bp, Artificial Sequence
  Synthetic nucleic acid sequcne
ggagaatgattagcacggagagtgg

SEQ ID NO: 4 - DNA, 25 bp, Artificial Sequence
  Synthetic nucleic acid sequcne
aacgattgttatgcccctaactcag

SEQ ID NO: 5 - DNA, 25 bp, Artificial Sequence
  Synthetic nucleic acid sequcne
atggacgacttgggacgggtatcaa

SEQ ID NO: 6 - DNA, 25 bp, Artificial Sequence
  Synthetic nucleic acid sequcne
taatgtggcttggctcaccgctagg

SEQ ID NO: 7 - DNA, 25 bp, Artificial Sequence
  Synthetic nucleic acid sequcne
cgatgtagtcatcccgatgtgctgg